SEQUENCE LISTING (368 sequences)

SEQ ID NO: 1 - DNA, 1020 bp, Homo sapiens
atgtctactgttcacgaaatcctgtgcaagctcagcttggagggtgatcactctacacccccaagtgcatatgggtctgtcaaagcctatactaactttgatgctgagcgggatgctttgaacattgaaacagccatcaagaccaaaggtgtggatgaggtcaccattgtcaacattttgaccaaccgcagcaatgcacagagacaggatattgccttcgcctaccagagaaggaccaaaaaggaacttgcatcagcactgaagtcagccttatctggccacctggagacggtgattttgggcctattgaagacacctgctcagtatgacgcttctgagctaaaagcttccatgaaggggctgggaaccgacgaggactctctcattgagatcatctgctccagaaccaaccaggagctgcaggaaattaacagagtctacaaggaaatgtacaagactgatctggagaaggacattatttcggacacatctggtgacttccgcaagctgatggttgccctggcaaagggtagaagagcagaggatggctctgtcattgattatgaactgattgaccaagatgctcgggatctctatgacgctggagtgaagaggaaaggaactgatgttcccaagtggatcagcatcatgaccgagcggagcgtgccccacctccagaaagtatttgataggtacaagagttacagcccttatgacatgttggaaagcatcaggaaagaggttaaaggagacctggaaaatgctttcctgaacctggttcagtgcattcagaacaagcccctgtattttgctgatcggctgtatgactccatgaagggcaaggggacgcgagataaggtcctgatcagaatcatggtctcccgcagtgaagtggacatgttgaaaattaggtctgaattcaagagaaagtacggcaagtccctgtactattatatccagcaagacactaagggcgactaccagaaagcgctgctgtacctgtgtggtggagatgactga

SEQ ID NO: 2 - protein, 1020 aa, Homo sapiens
ATGTCTACTGTTCACGAAATCCTGTGCAAGCTCAGCTTGGAGGGTGATCACTCTACACCCCCAAGTGCATATGGGTCTGTCAAAGCCTATACTAACTTTGATGCTGAGCGGGATGCTTTGAACATTGAAACAGCCATCAAGACCAAAGGTGTGGATGAGGTCACCATTGTCAACATTTTGACCAACCGCAGCAATGCACAGAGACAGGATATTGCCTTCGCCTACCAGAGAAGGACCAAAAAGGAACTTGCATCAGCACTGAAGTCAGCCTTATCTGGCCACCTGGAGACGGTGATTTTGGGCCTATTGAAGACACCTGCTCAGTATGACGCTTCTGAGCTAAAAGCTTCCATGAAGGGGCTGGGAACCGACGAGGACTCTCTCATTGAGATCATCTGCTCCAGAACCAACCAGGAGCTGCAGGAAATTAACAGAGTCTACAAGGAAATGTACAAGACTGATCTGGAGAAGGACATTATTTCGGACACATCTGGTGACTTCCGCAAGCTGATGGTTGCCCTGGCAAAGGGTAGAAGAGCAGAGGATGGCTCTGTCATTGATTATGAACTGATTGACCAAGATGCTCGGGATCTCTATGACGCTGGAGTGAAGAGGAAAGGAACTGATGTTCCCAAGTGGATCAGCATCATGACCGAGCGGAGCGTGCCCCACCTCCAGAAAGTATTTGATAGGTACAAGAGTTACAGCCCTTATGACATGTTGGAAAGCATCAGGAAAGAGGTTAAAGGAGACCTGGAAAATGCTTTCCTGAACCTGGTTCAGTGCATTCAGAACAAGCCCCTGTATTTTGCTGATCGGCTGTATGACTCCATGAAGGGCAAGGGGACGCGAGATAAGGTCCTGATCAGAATCATGGTCTCCCGCAGTGAAGTGGACATGTTGAAAATTAGGTCTGAATTCAAGAGAAAGTACGGCAAGTCCCTGTACTATTATATCCAGCAAGACACTAAGGGCGACTACCAGAAAGCGCTGCTGTACCTGTGTGGTGGAGATGACTGA

SEQ ID NO: 3 - DNA, 139 bp, Homo sapiens
cagggatttgccatatttcctcttgaattcagatctgattttcaacatgtccacttcactgcgagagaccatgattctaatcaggaccttgtctcgagtccccttgcccttcatggagtcatacagccggtcagcaaag

SEQ ID NO: 4 - DNA, 376 bp, Homo sapiens
ctgagcaggtgtcttcaacaggcctaacatcacggtctccaggtgaccagacaaggccgacttcatcgccgatggcagttcctttttggtcctcctctggtaggcgaaggcaatgtcctgcctctgtgcattgctgcggttagtcagaatgttgacaatggtgacctcgtccacgcctttggtcttgattgctgtttcaatgttcaaagcatccctctcagcgtcgaagttggtgtagggtttgaccgacccataggcacttgggggtgtagaatgatcaccctccaagctgagcttgcacaggatttcgtggacagtagacattttgaaaaaaaagctgggccgggcacctattgcagacctgcccgggcggccg

SEQ ID NO: 5 - DNA, 139 bp, Homo sapiens
ctttgctgaccggctgtatgactccatgaagggcaaggggactcgagacaaggtcctgattagaatcatggtctctcgcagtgaagtggacatgttgaaaatcagatctgaattcaagaggaaatatggcaaatccctg

SEQ ID NO: 6 - DNA, 412 bp, Homo sapiens
acctggagaccgtgatgttaggcctgttgaagacacctgctcagtacgatgcctctgagctcaaagcctccatgaagggcctggggactgatgaggactccctcatcgagatcatctgctcaagaaccaaccaggagctgcaggagattaaccgagtgtataaggaaatgtacaagaccgatctggagaaggacatcatctctgacacatctggagaattccgaaagctgttggtcgcccttgcaaaagggttaacgggcagaggatggttctgttattgactacgagctgattgaccaggatgcccgggagctctatgatgctggggtgaagaggaaaggaaccgatgtccccaagtggatcagcatcatgactgaacgcagtgtgtgccacctccagaaagtgttcgaaa

SEQ ID NO: 7 - RNA, 19 nt, Homo sapiens
cuuugaugcugagcgggau

SEQ ID NO: 8 - RNA, 19 nt, Homo sapiens
gaccgaucuggagaaggac

SEQ ID NO: 9 - RNA, 19 nt, Homo sapiens
ucaagaccaaaggcgugga

SEQ ID NO: 10 - RNA, 19 nt, Homo sapiens
ccaaccaggagcugcagga

SEQ ID NO: 11 - RNA, 19 nt, Homo sapiens
cugauugaccaagaugcuc

SEQ ID NO: 12 - RNA, 19 nt, Homo sapiens
aucccgcucagcaucaaag

SEQ ID NO: 13 - RNA, 19 nt, Homo sapiens
guccuucuccagaucgguc

SEQ ID NO: 14 - RNA, 19 nt, Homo sapiens
uccacgccuuuggucuuga

SEQ ID NO: 15 - RNA, 19 nt, Homo sapiens
uccugcagcuccugguugg

SEQ ID NO: 16 - RNA, 19 nt, Homo sapiens
gagcaucuuggucaaucag

SEQ ID NO: 17 - RNA, 19 nt, Homo sapiens
caagcucagcuuggagggu

SEQ ID NO: 18 - RNA, 19 nt, Homo sapiens
ggaugcuuugaacauugaa

SEQ ID NO: 19 - RNA, 19 nt, Homo sapiens
uuccugaaccugguucagu

SEQ ID NO: 20 - RNA, 19 nt, Homo sapiens
uccagcaagacacuaaggg

SEQ ID NO: 21 - RNA, 19 nt, Homo sapiens
ccugguucagugcauucag

SEQ ID NO: 22 - RNA, 19 nt, Homo sapiens
auccugugcaagcucagcu

SEQ ID NO: 23 - RNA, 19 nt, Homo sapiens
aaguggacauguugaaaau

SEQ ID NO: 24 - RNA, 19 nt, Homo sapiens
auugccuucgccuaccaga

SEQ ID NO: 25 - RNA, 19 nt, Homo sapiens
guucagugcauucagaaca

SEQ ID NO: 26 - RNA, 19 nt, Homo sapiens
ccaaagaaaugaacauucc

SEQ ID NO: 27 - RNA, 19 nt, Homo sapiens
uccugaaccugguucagug

SEQ ID NO: 28 - RNA, 19 nt, Homo sapiens
ugacuccaugaagggcaag

SEQ ID NO: 29 - RNA, 19 nt, Homo sapiens
ugaaguggacauguugaaa

SEQ ID NO: 30 - RNA, 19 nt, Homo sapiens
agugaaguggacauguuga

SEQ ID NO: 31 - RNA, 19 nt, Homo sapiens
ugaagacaccugcucagua

SEQ ID NO: 32 - RNA, 19 nt, Homo sapiens
accgcagcaaugcacagag

SEQ ID NO: 33 - RNA, 19 nt, Homo sapiens
gaaguggacauguugaaaa

SEQ ID NO: 34 - RNA, 19 nt, Homo sapiens
gugcaagcucagcuuggag

SEQ ID NO: 35 - RNA, 19 nt, Homo sapiens
gugaaguggacauguugaa

SEQ ID NO: 36 - RNA, 19 nt, Homo sapiens
ggcuguaugacuccaugaa

SEQ ID NO: 37 - RNA, 19 nt, Homo sapiens
cggcuguaugacuccauga

SEQ ID NO: 38 - RNA, 19 nt, Homo sapiens
ugaaccugguucagugcau

SEQ ID NO: 39 - RNA, 19 nt, Homo sapiens
ugguucagugcauucagaa

SEQ ID NO: 40 - RNA, 19 nt, Homo sapiens
cuguaugacuccaugaagg

SEQ ID NO: 41 - RNA, 19 nt, Homo sapiens
accugguucagugcauuca

SEQ ID NO: 42 - RNA, 19 nt, Homo sapiens
ugcaagcucagcuuggagg

SEQ ID NO: 43 - RNA, 19 nt, Homo sapiens
cgcagugaaguggacaugu

SEQ ID NO: 44 - RNA, 19 nt, Homo sapiens
uugccuucgccuaccagag

SEQ ID NO: 45 - RNA, 19 nt, Homo sapiens
ccugaaccugguucagugc

SEQ ID NO: 46 - RNA, 19 nt, Homo sapiens
cucagcuuggagggugauc

SEQ ID NO: 47 - RNA, 19 nt, Homo sapiens
gccaaagaaaugaacauuc

SEQ ID NO: 48 - RNA, 19 nt, Homo sapiens
gcucagcuuggagggugau

SEQ ID NO: 49 - RNA, 19 nt, Homo sapiens
gaaccugguucagugcauu

SEQ ID NO: 50 - RNA, 19 nt, Homo sapiens
gcuguaugacuccaugaag

SEQ ID NO: 51 - RNA, 19 nt, Homo sapiens
gcagugaaguggacauguu

SEQ ID NO: 52 - RNA, 19 nt, Homo sapiens
guaugacuccaugaagggc

SEQ ID NO: 53 - RNA, 19 nt, Homo sapiens
gugcauucagaacaagccc

SEQ ID NO: 54 - RNA, 19 nt, Homo sapiens
uaugacuccaugaagggca

SEQ ID NO: 55 - RNA, 19 nt, Homo sapiens
gggaugcuuugaacauuga

SEQ ID NO: 56 - RNA, 19 nt, Homo sapiens
uccugugcaagcucagcuu

SEQ ID NO: 57 - RNA, 19 nt, Homo sapiens
gguucagugcauucagaac

SEQ ID NO: 58 - RNA, 19 nt, Homo sapiens
ugcauucagaacaagcccc

SEQ ID NO: 59 - RNA, 19 nt, Homo sapiens
aaccaaccaggagcugcag

SEQ ID NO: 60 - RNA, 19 nt, Homo sapiens
cugugcaagcucagcuugg

SEQ ID NO: 61 - RNA, 19 nt, Homo sapiens
uucagaacaagccccugua

SEQ ID NO: 62 - RNA, 19 nt, Homo sapiens
cagugcauucagaacaagc

SEQ ID NO: 63 - RNA, 19 nt, Homo sapiens
uguaugacuccaugaaggg

SEQ ID NO: 64 - RNA, 19 nt, Homo sapiens
gcuuugaacauugaaacag

SEQ ID NO: 65 - RNA, 19 nt, Homo sapiens
cugaaccugguucagugca

SEQ ID NO: 66 - RNA, 19 nt, Homo sapiens
cagugaaguggacauguug

SEQ ID NO: 67 - RNA, 19 nt, Homo sapiens
auccagcaagacacuaagg

SEQ ID NO: 68 - RNA, 19 nt, Homo sapiens
aaccugguucagugcauuc

SEQ ID NO: 69 - RNA, 19 nt, Homo sapiens
caaagaaaugaacauucca

SEQ ID NO: 70 - RNA, 19 nt, Homo sapiens
cauucagaacaagccccug

SEQ ID NO: 71 - RNA, 19 nt, Homo sapiens
aagcucagcuuggagggug

SEQ ID NO: 72 - RNA, 19 nt, Homo sapiens
ucagcuuggagggugauca

SEQ ID NO: 73 - RNA, 19 nt, Homo sapiens
agaaccaaccaggagcugc

SEQ ID NO: 74 - RNA, 19 nt, Homo sapiens
ugcuuugaacauugaaaca

SEQ ID NO: 75 - RNA, 19 nt, Homo sapiens
agcucagcuuggaggguga

SEQ ID NO: 76 - RNA, 19 nt, Homo sapiens
aaccgcagcaaugcacaga

SEQ ID NO: 77 - RNA, 19 nt, Homo sapiens
agugcauucagaacaagcc

SEQ ID NO: 78 - RNA, 19 nt, Homo sapiens
ugugcaagcucagcuugga

SEQ ID NO: 79 - RNA, 19 nt, Homo sapiens
ggucaccauugucaacauu

SEQ ID NO: 80 - RNA, 19 nt, Homo sapiens
augacuccaugaagggcaa

SEQ ID NO: 81 - RNA, 19 nt, Homo sapiens
gcugcuguaccuguguggu

SEQ ID NO: 82 - RNA, 19 nt, Homo sapiens
uugaagacaccugcucagu

SEQ ID NO: 83 - RNA, 19 nt, Homo sapiens
augcuuugaacauugaaac

SEQ ID NO: 84 - RNA, 19 nt, Homo sapiens
gcauucagaacaagccccu

SEQ ID NO: 85 - RNA, 19 nt, Homo sapiens
uucagugcauucagaacaa

SEQ ID NO: 86 - RNA, 19 nt, Homo sapiens
cugguucagugcauucaga

SEQ ID NO: 87 - RNA, 19 nt, Homo sapiens
cuuugaacauugaaacagc

SEQ ID NO: 88 - RNA, 19 nt, Homo sapiens
ucagugcauucagaacaag

SEQ ID NO: 89 - RNA, 19 nt, Homo sapiens
gaugcuuugaacauugaaa

SEQ ID NO: 90 - RNA, 19 nt, Homo sapiens
auucagaacaagccccugu

SEQ ID NO: 91 - RNA, 19 nt, Homo sapiens
ccugugcaagcucagcuug

SEQ ID NO: 92 - RNA, 19 nt, Homo sapiens
caaguggaucagcaucaug

SEQ ID NO: 93 - RNA, 19 nt, Homo sapiens
aaauccugugcaagcucag

SEQ ID NO: 94 - RNA, 19 nt, Homo sapiens
cacgaaauccugugcaagc

SEQ ID NO: 95 - RNA, 19 nt, Homo sapiens
uccaugaagggcaagggga

SEQ ID NO: 96 - RNA, 19 nt, Homo sapiens
acgaaauccugugcaagcu

SEQ ID NO: 97 - RNA, 19 nt, Homo sapiens
gacuccaugaagggcaagg

SEQ ID NO: 98 - RNA, 19 nt, Homo sapiens
aaguggaucagcaucauga

SEQ ID NO: 99 - RNA, 19 nt, Homo sapiens
cccaaguggaucagcauca

SEQ ID NO: 100 - RNA, 19 nt, Homo sapiens
gaggucaccauugucaaca

SEQ ID NO: 101 - RNA, 19 nt, Homo sapiens
cugcuguaccuguguggug

SEQ ID NO: 102 - RNA, 19 nt, Homo sapiens
aggucaccauugucaacau

SEQ ID NO: 103 - RNA, 19 nt, Homo sapiens
aguggaucagcaucaugac

SEQ ID NO: 104 - RNA, 19 nt, Homo sapiens
aauccugugcaagcucagc

SEQ ID NO: 105 - RNA, 19 nt, Homo sapiens
acuccaugaagggcaaggg

SEQ ID NO: 106 - RNA, 19 nt, Homo sapiens
cgaaauccugugcaagcuc

SEQ ID NO: 107 - RNA, 19 nt, Homo sapiens
ccaaguggaucagcaucau

SEQ ID NO: 108 - RNA, 19 nt, Homo sapiens
gaaauccugugcaagcuca

SEQ ID NO: 109 - RNA, 19 nt, Homo sapiens
ugcuguaccuguguggugg

SEQ ID NO: 110 - RNA, 19 nt, Homo sapiens
gaaccaaccaggagcugca

SEQ ID NO: 111 - RNA, 19 nt, Homo sapiens
gcaagucccuguacuauua

SEQ ID NO: 112 - RNA, 19 nt, Homo sapiens
ggauggcucugucauugau

SEQ ID NO: 113 - RNA, 19 nt, Homo sapiens
ccaaggaguuggaagugaa

SEQ ID NO: 114 - RNA, 19 nt, Homo sapiens
ggagggugaucacucuaca

SEQ ID NO: 115 - RNA, 19 nt, Homo sapiens
gaagugaagucuaugaugu

SEQ ID NO: 116 - RNA, 19 nt, Homo sapiens
ggaucagcaucaugaccga

SEQ ID NO: 117 - RNA, 19 nt, Homo sapiens
ggacugagcuguacaguau

SEQ ID NO: 118 - RNA, 19 nt, Homo sapiens
gaagacaccugcucaguau

SEQ ID NO: 119 - RNA, 19 nt, Homo sapiens
acccuccaagcugagcuug

SEQ ID NO: 120 - RNA, 19 nt, Homo sapiens
uucaauguucaaagcaucc

SEQ ID NO: 121 - RNA, 19 nt, Homo sapiens
acugaaccagguucaggaa

SEQ ID NO: 122 - RNA, 19 nt, Homo sapiens
cccuuagugucuugcugga

SEQ ID NO: 123 - RNA, 19 nt, Homo sapiens
cugaaugcacugaaccagg

SEQ ID NO: 124 - RNA, 19 nt, Homo sapiens
agcugagcuugcacaggau

SEQ ID NO: 125 - RNA, 19 nt, Homo sapiens
auuuucaacauguccacuu

SEQ ID NO: 126 - RNA, 19 nt, Homo sapiens
ucugguaggcgaaggcaau

SEQ ID NO: 127 - RNA, 19 nt, Homo sapiens
uguucugaaugcacugaac

SEQ ID NO: 128 - RNA, 19 nt, Homo sapiens
ggaauguucauuucuuugg

SEQ ID NO: 129 - RNA, 19 nt, Homo sapiens
cacugaaccagguucagga

SEQ ID NO: 130 - RNA, 19 nt, Homo sapiens
cuugcccuucauggaguca

SEQ ID NO: 131 - RNA, 19 nt, Homo sapiens
uuucaacauguccacuuca

SEQ ID NO: 132 - RNA, 19 nt, Homo sapiens
ucaacauguccacuucacu

SEQ ID NO: 133 - RNA, 19 nt, Homo sapiens
uacugagcaggugucuuca

SEQ ID NO: 134 - RNA, 19 nt, Homo sapiens
cucugugcauugcugcggu

SEQ ID NO: 135 - RNA, 19 nt, Homo sapiens
uuuucaacauguccacuuc

SEQ ID NO: 136 - RNA, 19 nt, Homo sapiens
cuccaagcugagcuugcac

SEQ ID NO: 137 - RNA, 19 nt, Homo sapiens
uucaacauguccacuucac

SEQ ID NO: 138 - RNA, 19 nt, Homo sapiens
uucauggagucauacagcc

SEQ ID NO: 139 - RNA, 19 nt, Homo sapiens
ucauggagucauacagccg

SEQ ID NO: 140 - RNA, 19 nt, Homo sapiens
augcacugaaccagguuca

SEQ ID NO: 141 - RNA, 19 nt, Homo sapiens
uucugaaugcacugaacca

SEQ ID NO: 142 - RNA, 19 nt, Homo sapiens
ccuucauggagucauacag

SEQ ID NO: 143 - RNA, 19 nt, Homo sapiens
ugaaugcacugaaccaggu

SEQ ID NO: 144 - RNA, 19 nt, Homo sapiens
ccuccaagcugagcuugca

SEQ ID NO: 145 - RNA, 19 nt, Homo sapiens
acauguccacuucacugcg

SEQ ID NO: 146 - RNA, 19 nt, Homo sapiens
cucugguaggcgaaggcaa

SEQ ID NO: 147 - RNA, 19 nt, Homo sapiens
gcacugaaccagguucagg

SEQ ID NO: 148 - RNA, 19 nt, Homo sapiens
gaucacccuccaagcugag

SEQ ID NO: 149 - RNA, 19 nt, Homo sapiens
gaauguucauuucuuuggc

SEQ ID NO: 150 - RNA, 19 nt, Homo sapiens
aucacccuccaagcugagc

SEQ ID NO: 151 - RNA, 19 nt, Homo sapiens
aaugcacugaaccagguuc

SEQ ID NO: 152 - RNA, 19 nt, Homo sapiens
cuucauggagucauacagc

SEQ ID NO: 153 - RNA, 19 nt, Homo sapiens
aacauguccacuucacugc

SEQ ID NO: 154 - RNA, 19 nt, Homo sapiens
gcccuucauggagucauac

SEQ ID NO: 155 - RNA, 19 nt, Homo sapiens
gggcuuguucugaaugcac

SEQ ID NO: 156 - RNA, 19 nt, Homo sapiens
ugcccuucauggagucaua

SEQ ID NO: 157 - RNA, 19 nt, Homo sapiens
ucaauguucaaagcauccc

SEQ ID NO: 158 - RNA, 19 nt, Homo sapiens
aagcugagcuugcacagga

SEQ ID NO: 159 - RNA, 19 nt, Homo sapiens
guucugaaugcacugaacc

SEQ ID NO: 160 - RNA, 19 nt, Homo sapiens
ggggcuuguucugaaugca

SEQ ID NO: 161 - RNA, 19 nt, Homo sapiens
cugcagcuccugguugguu

SEQ ID NO: 162 - RNA, 19 nt, Homo sapiens
ccaagcugagcuugcacag

SEQ ID NO: 163 - RNA, 19 nt, Homo sapiens
uacaggggcuuguucugaa

SEQ ID NO: 164 - RNA, 19 nt, Homo sapiens
gcuuguucugaaugcacug

SEQ ID NO: 165 - RNA, 19 nt, Homo sapiens
cccuucauggagucauaca

SEQ ID NO: 166 - RNA, 19 nt, Homo sapiens
cuguuucaauguucaaagc

SEQ ID NO: 167 - RNA, 19 nt, Homo sapiens
ugcacugaaccagguucag

SEQ ID NO: 168 - RNA, 19 nt, Homo sapiens
caacauguccacuucacug

SEQ ID NO: 169 - RNA, 19 nt, Homo sapiens
ccuuagugucuugcuggau

SEQ ID NO: 170 - RNA, 19 nt, Homo sapiens
gaaugcacugaaccagguu

SEQ ID NO: 171 - RNA, 19 nt, Homo sapiens
uggaauguucauuucuuug

SEQ ID NO: 172 - RNA, 19 nt, Homo sapiens
caggggcuuguucugaaug

SEQ ID NO: 173 - RNA, 19 nt, Homo sapiens
cacccuccaagcugagcuu

SEQ ID NO: 174 - RNA, 19 nt, Homo sapiens
ugaucacccuccaagcuga

SEQ ID NO: 175 - RNA, 19 nt, Homo sapiens
gcagcuccugguugguucu

SEQ ID NO: 176 - RNA, 19 nt, Homo sapiens
uguuucaauguucaaagca

SEQ ID NO: 177 - RNA, 19 nt, Homo sapiens
ucacccuccaagcugagcu

SEQ ID NO: 178 - RNA, 19 nt, Homo sapiens
ucugugcauugcugcgguu

SEQ ID NO: 179 - RNA, 19 nt, Homo sapiens
ggcuuguucugaaugcacu

SEQ ID NO: 180 - RNA, 19 nt, Homo sapiens
uccaagcugagcuugcaca

SEQ ID NO: 181 - RNA, 19 nt, Homo sapiens
aauguugacaauggugacc

SEQ ID NO: 182 - RNA, 19 nt, Homo sapiens
uugcccuucauggagucau

SEQ ID NO: 183 - RNA, 19 nt, Homo sapiens
accacacagguacagcagc

SEQ ID NO: 184 - RNA, 19 nt, Homo sapiens
acugagcaggugucuucaa

SEQ ID NO: 185 - RNA, 19 nt, Homo sapiens
guuucaauguucaaagcau

SEQ ID NO: 186 - RNA, 19 nt, Homo sapiens
aggggcuuguucugaaugc

SEQ ID NO: 187 - RNA, 19 nt, Homo sapiens
uuguucugaaugcacugaa

SEQ ID NO: 188 - RNA, 19 nt, Homo sapiens
ucugaaugcacugaaccag

SEQ ID NO: 189 - RNA, 19 nt, Homo sapiens
gcuguuucaauguucaaag

SEQ ID NO: 190 - RNA, 19 nt, Homo sapiens
cuuguucugaaugcacuga

SEQ ID NO: 191 - RNA, 19 nt, Homo sapiens
uuucaauguucaaagcauc

SEQ ID NO: 192 - RNA, 19 nt, Homo sapiens
acaggggcuuguucugaau

SEQ ID NO: 193 - RNA, 19 nt, Homo sapiens
caagcugagcuugcacagg

SEQ ID NO: 194 - RNA, 19 nt, Homo sapiens
caugaugcugauccacuug

SEQ ID NO: 195 - RNA, 19 nt, Homo sapiens
cugagcuugcacaggauuu

SEQ ID NO: 196 - RNA, 19 nt, Homo sapiens
gcuugcacaggauuucgug

SEQ ID NO: 197 - RNA, 19 nt, Homo sapiens
uccccuugcccuucaugga

SEQ ID NO: 198 - RNA, 19 nt, Homo sapiens
agcuugcacaggauuucgu

SEQ ID NO: 199 - RNA, 19 nt, Homo sapiens
ccuugcccuucauggaguc

SEQ ID NO: 200 - RNA, 19 nt, Homo sapiens
ucaugaugcugauccacuu

SEQ ID NO: 201 - RNA, 19 nt, Homo sapiens
ugaugcugauccacuuggg

SEQ ID NO: 202 - RNA, 19 nt, Homo sapiens
uguugacaauggugaccuc

SEQ ID NO: 203 - RNA, 19 nt, Homo sapiens
caccacacagguacagcag

SEQ ID NO: 204 - RNA, 19 nt, Homo sapiens
auguugacaauggugaccu

SEQ ID NO: 205 - RNA, 19 nt, Homo sapiens
gucaugaugcugauccacu

SEQ ID NO: 206 - RNA, 19 nt, Homo sapiens
gcugagcuugcacaggauu

SEQ ID NO: 207 - RNA, 19 nt, Homo sapiens
cccuugcccuucauggagu

SEQ ID NO: 208 - RNA, 19 nt, Homo sapiens
gagcuugcacaggauuucg

SEQ ID NO: 209 - RNA, 19 nt, Homo sapiens
augaugcugauccacuugg

SEQ ID NO: 210 - RNA, 19 nt, Homo sapiens
ugagcuugcacaggauuuc

SEQ ID NO: 211 - RNA, 19 nt, Homo sapiens
ccaccacacagguacagca

SEQ ID NO: 212 - RNA, 19 nt, Homo sapiens
ugcagcuccugguugguuc

SEQ ID NO: 213 - RNA, 19 nt, Homo sapiens
uaauaguacagggacuugc

SEQ ID NO: 214 - RNA, 19 nt, Homo sapiens
aucaaugacagagccaucc

SEQ ID NO: 215 - RNA, 19 nt, Homo sapiens
uucacuuccaacuccuugg

SEQ ID NO: 216 - RNA, 19 nt, Homo sapiens
uguagagugaucacccucc

SEQ ID NO: 217 - RNA, 19 nt, Homo sapiens
acaucauagacuucacuuc

SEQ ID NO: 218 - RNA, 19 nt, Homo sapiens
ucggucaugaugcugaucc

SEQ ID NO: 219 - RNA, 19 nt, Homo sapiens
auacuguacagcucagucc

SEQ ID NO: 220 - RNA, 19 nt, Homo sapiens
auacugagcaggugucuuc

SEQ ID NO: 221 - RNA, 21 nt, Homo sapiens
aacugccaucggcgaugaagu

SEQ ID NO: 222 - RNA, 21 nt, Homo sapiens
cgaaauccugugcaagcucag

SEQ ID NO: 223 - RNA, 21 nt, Homo sapiens
gaaauccugugcaagcucagc

SEQ ID NO: 224 - RNA, 21 nt, Homo sapiens
acuccaugaagggcaagggga

SEQ ID NO: 225 - RNA, 21 nt, Homo sapiens
cccaaguggaucagcaucaug

SEQ ID NO: 226 - RNA, 21 nt, Homo sapiens
caaguggaucagcaucaugac

SEQ ID NO: 227 - RNA, 21 nt, Homo sapiens
cacgaaauccugugcaagcuc

SEQ ID NO: 228 - RNA, 21 nt, Homo sapiens
ccaaguggaucagcaucauga

SEQ ID NO: 229 - RNA, 21 nt, Homo sapiens
acgaaauccugugcaagcuca

SEQ ID NO: 230 - RNA, 21 nt, Homo sapiens
ugacuccaugaagggcaaggg

SEQ ID NO: 231 - RNA, 21 nt, Homo sapiens
cugaaccugguucagugcauu

SEQ ID NO: 232 - RNA, 21 nt, Homo sapiens
accugguucagugcauucaga

SEQ ID NO: 233 - RNA, 21 nt, Homo sapiens
ugaaccugguucagugcauuc

SEQ ID NO: 234 - RNA, 21 nt, Homo sapiens
ggcuguaugacuccaugaagg

SEQ ID NO: 235 - RNA, 21 nt, Homo sapiens
ugcauucagaacaagccccug

SEQ ID NO: 236 - RNA, 21 nt, Homo sapiens
cuguaugacuccaugaagggc

SEQ ID NO: 237 - RNA, 21 nt, Homo sapiens
gcauucagaacaagccccugu

SEQ ID NO: 238 - RNA, 21 nt, Homo sapiens
gaaccugguucagugcauuca

SEQ ID NO: 239 - RNA, 21 nt, Homo sapiens
cugguucagugcauucagaac

SEQ ID NO: 240 - RNA, 21 nt, Homo sapiens
agcucagcuuggagggugauc

SEQ ID NO: 241 - RNA, 21 nt, Homo sapiens
guaugacuccaugaagggcaa

SEQ ID NO: 242 - RNA, 21 nt, Homo sapiens
gugcauucagaacaagccccu

SEQ ID NO: 243 - RNA, 21 nt, Homo sapiens
guucagugcauucagaacaag

SEQ ID NO: 244 - RNA, 21 nt, Homo sapiens
aagcucagcuuggagggugau

SEQ ID NO: 245 - RNA, 21 nt, Homo sapiens
agugcauucagaacaagcccc

SEQ ID NO: 246 - RNA, 21 nt, Homo sapiens
auccugugcaagcucagcuug

SEQ ID NO: 247 - RNA, 21 nt, Homo sapiens
agaaccaaccaggagcugcag

SEQ ID NO: 248 - RNA, 21 nt, Homo sapiens
uccugugcaagcucagcuugg

SEQ ID NO: 249 - RNA, 21 nt, Homo sapiens
ccugugcaagcucagcuugga

SEQ ID NO: 250 - RNA, 21 nt, Homo sapiens
uuccugaaccugguucagugc

SEQ ID NO: 251 - RNA, 21 nt, Homo sapiens
gcagugaaguggacauguuga

SEQ ID NO: 252 - RNA, 21 nt, Homo sapiens
aauccugugcaagcucagcuu

SEQ ID NO: 253 - RNA, 21 nt, Homo sapiens
ugcuuugaacauugaaacagc

SEQ ID NO: 254 - RNA, 21 nt, Homo sapiens
ugcaagcucagcuuggagggu

SEQ ID NO: 255 - RNA, 21 nt, Homo sapiens
uccugaaccugguucagugca

SEQ ID NO: 256 - RNA, 21 nt, Homo sapiens
cagugaaguggacauguugaa

SEQ ID NO: 257 - RNA, 21 nt, Homo sapiens
ugaaguggacauguugaaaau

SEQ ID NO: 258 - RNA, 21 nt, Homo sapiens
uucagugcauucagaacaagc

SEQ ID NO: 259 - RNA, 21 nt, Homo sapiens
ugguucagugcauucagaaca

SEQ ID NO: 260 - RNA, 21 nt, Homo sapiens
ucagugcauucagaacaagcc

SEQ ID NO: 261 - RNA, 21 nt, Homo sapiens
gugaaguggacauguugaaaa

SEQ ID NO: 262 - RNA, 21 nt, Homo sapiens
cggcuguaugacuccaugaag

SEQ ID NO: 263 - RNA, 21 nt, Homo sapiens
gaugcuuugaacauugaaaca

SEQ ID NO: 264 - RNA, 21 nt, Homo sapiens
augacuccaugaagggcaagg

SEQ ID NO: 265 - RNA, 21 nt, Homo sapiens
cagugcauucagaacaagccc

SEQ ID NO: 266 - RNA, 21 nt, Homo sapiens
uaugacuccaugaagggcaag

SEQ ID NO: 267 - RNA, 21 nt, Homo sapiens
ggaugcuuugaacauugaaac

SEQ ID NO: 268 - RNA, 21 nt, Homo sapiens
gaggucaccauugucaacauu

SEQ ID NO: 269 - RNA, 21 nt, Homo sapiens
aaccaaccaggagcugcagga

SEQ ID NO: 270 - RNA, 21 nt, Homo sapiens
gguucagugcauucagaacaa

SEQ ID NO: 271 - RNA, 21 nt, Homo sapiens
gggaugcuuugaacauugaaa

SEQ ID NO: 272 - RNA, 21 nt, Homo sapiens
cauucagaacaagccccugua

SEQ ID NO: 273 - RNA, 21 nt, Homo sapiens
gcuguaugacuccaugaaggg

SEQ ID NO: 274 - RNA, 21 nt, Homo sapiens
ugugcaagcucagcuuggagg

SEQ ID NO: 275 - RNA, 21 nt, Homo sapiens
cgcagugaaguggacauguug

SEQ ID NO: 276 - RNA, 21 nt, Homo sapiens
ccugaaccugguucagugcau

SEQ ID NO: 277 - RNA, 21 nt, Homo sapiens
ccugguucagugcauucagaa

SEQ ID NO: 278 - RNA, 21 nt, Homo sapiens
gccaaagaaaugaacauucca

SEQ ID NO: 279 - RNA, 21 nt, Homo sapiens
gcucagcuuggagggugauca

SEQ ID NO: 280 - RNA, 21 nt, Homo sapiens
uguaugacuccaugaagggca

SEQ ID NO: 281 - RNA, 21 nt, Homo sapiens
aaccugguucagugcauucag

SEQ ID NO: 282 - RNA, 21 nt, Homo sapiens
cugugcaagcucagcuuggag

SEQ ID NO: 283 - RNA, 21 nt, Homo sapiens
caagcucagcuuggaggguga

SEQ ID NO: 284 - RNA, 21 nt, Homo sapiens
aaauccugugcaagcucagcu

SEQ ID NO: 285 - RNA, 21 nt, Homo sapiens
augcuuugaacauugaaacag

SEQ ID NO: 286 - RNA, 21 nt, Homo sapiens
agugaaguggacauguugaaa

SEQ ID NO: 287 - RNA, 21 nt, Homo sapiens
ggauggcucugucauugauua

SEQ ID NO: 288 - RNA, 21 nt, Homo sapiens
ccaaagaaaugaacauuccaa

SEQ ID NO: 289 - RNA, 21 nt, Homo sapiens
gagauaagguccugaucagaa

SEQ ID NO: 290 - RNA, 21 nt, Homo sapiens
gcaagucccuguacuauuaua

SEQ ID NO: 291 - RNA, 21 nt, Homo sapiens
cgaggacucucucauugagau

SEQ ID NO: 292 - RNA, 21 nt, Homo sapiens
ccaaggaguuggaagugaagu

SEQ ID NO: 293 - RNA, 21 nt, Homo sapiens
gaagugaagucuaugauguga

SEQ ID NO: 294 - RNA, 21 nt, Homo sapiens
gaagacaccugcucaguauga

SEQ ID NO: 295 - RNA, 21 nt, Homo sapiens
acuucaucgccgauggcaguu

SEQ ID NO: 296 - RNA, 21 nt, Homo sapiens
cugagcuugcacaggauuucg

SEQ ID NO: 297 - RNA, 21 nt, Homo sapiens
gcugagcuugcacaggauuuc

SEQ ID NO: 298 - RNA, 21 nt, Homo sapiens
uccccuugcccuucauggagu

SEQ ID NO: 299 - RNA, 21 nt, Homo sapiens
caugaugcugauccacuuggg

SEQ ID NO: 300 - RNA, 21 nt, Homo sapiens
gucaugaugcugauccacuug

SEQ ID NO: 301 - RNA, 21 nt, Homo sapiens
gagcuugcacaggauuucgug

SEQ ID NO: 302 - RNA, 21 nt, Homo sapiens
ucaugaugcugauccacuugg

SEQ ID NO: 303 - RNA, 21 nt, Homo sapiens
ugagcuugcacaggauuucgu

SEQ ID NO: 304 - RNA, 21 nt, Homo sapiens
cccuugcccuucauggaguca

SEQ ID NO: 305 - RNA, 21 nt, Homo sapiens
aaugcacugaaccagguucag

SEQ ID NO: 306 - RNA, 21 nt, Homo sapiens
ucugaaugcacugaaccaggu

SEQ ID NO: 307 - RNA, 21 nt, Homo sapiens
gaaugcacugaaccagguuca

SEQ ID NO: 308 - RNA, 21 nt, Homo sapiens
ccuucauggagucauacagcc

SEQ ID NO: 309 - RNA, 21 nt, Homo sapiens
caggggcuuguucugaaugca

SEQ ID NO: 310 - RNA, 21 nt, Homo sapiens
gcccuucauggagucauacag

SEQ ID NO: 311 - RNA, 21 nt, Homo sapiens
acaggggcuuguucugaaugc

SEQ ID NO: 312 - RNA, 21 nt, Homo sapiens
ugaaugcacugaaccagguuc

SEQ ID NO: 313 - RNA, 21 nt, Homo sapiens
guucugaaugcacugaaccag

SEQ ID NO: 314 - RNA, 21 nt, Homo sapiens
gaucacccuccaagcugagcu

SEQ ID NO: 315 - RNA, 21 nt, Homo sapiens
uugcccuucauggagucauac

SEQ ID NO: 316 - RNA, 21 nt, Homo sapiens
aggggcuuguucugaaugcac

SEQ ID NO: 317 - RNA, 21 nt, Homo sapiens
cuuguucugaaugcacugaac

SEQ ID NO: 318 - RNA, 21 nt, Homo sapiens
aucacccuccaagcugagcuu

SEQ ID NO: 319 - RNA, 21 nt, Homo sapiens
ggggcuuguucugaaugcacu

SEQ ID NO: 320 - RNA, 21 nt, Homo sapiens
caagcugagcuugcacaggau

SEQ ID NO: 321 - RNA, 21 nt, Homo sapiens
cugcagcuccugguugguucu

SEQ ID NO: 322 - RNA, 21 nt, Homo sapiens
ccaagcugagcuugcacagga

SEQ ID NO: 323 - RNA, 21 nt, Homo sapiens
uccaagcugagcuugcacagg

SEQ ID NO: 324 - RNA, 21 nt, Homo sapiens
gcacugaaccagguucaggaa

SEQ ID NO: 325 - RNA, 21 nt, Homo sapiens
ucaacauguccacuucacugc

SEQ ID NO: 326 - RNA, 21 nt, Homo sapiens
aagcugagcuugcacaggauu

SEQ ID NO: 327 - RNA, 21 nt, Homo sapiens
gcuguuucaauguucaaagca

SEQ ID NO: 328 - RNA, 21 nt, Homo sapiens
acccuccaagcugagcuugca

SEQ ID NO: 329 - RNA, 21 nt, Homo sapiens
ugcacugaaccagguucagga

SEQ ID NO: 330 - RNA, 21 nt, Homo sapiens
uucaacauguccacuucacug

SEQ ID NO: 331 - RNA, 21 nt, Homo sapiens
auuuucaacauguccacuuca

SEQ ID NO: 332 - RNA, 21 nt, Homo sapiens
gcuuguucugaaugcacugaa

SEQ ID NO: 333 - RNA, 21 nt, Homo sapiens
uguucugaaugcacugaacca

SEQ ID NO: 334 - RNA, 21 nt, Homo sapiens
ggcuuguucugaaugcacuga

SEQ ID NO: 335 - RNA, 21 nt, Homo sapiens
uuuucaacauguccacuucac

SEQ ID NO: 336 - RNA, 21 nt, Homo sapiens
cuucauggagucauacagccg

SEQ ID NO: 337 - RNA, 21 nt, Homo sapiens
uguuucaauguucaaagcauc

SEQ ID NO: 338 - RNA, 21 nt, Homo sapiens
ccuugcccuucauggagucau

SEQ ID NO: 339 - RNA, 21 nt, Homo sapiens
gggcuuguucugaaugcacug

SEQ ID NO: 340 - RNA, 21 nt, Homo sapiens
cuugcccuucauggagucaua

SEQ ID NO: 341 - RNA, 21 nt, Homo sapiens
guuucaauguucaaagcaucc

SEQ ID NO: 342 - RNA, 21 nt, Homo sapiens
aauguugacaauggugaccuc

SEQ ID NO: 343 - RNA, 21 nt, Homo sapiens
uccugcagcuccugguugguu

SEQ ID NO: 344 - RNA, 21 nt, Homo sapiens
uuguucugaaugcacugaacc

SEQ ID NO: 345 - RNA, 21 nt, Homo sapiens
uuucaauguucaaagcauccc

SEQ ID NO: 346 - RNA, 21 nt, Homo sapiens
uacaggggcuuguucugaaug

SEQ ID NO: 347 - RNA, 21 nt, Homo sapiens
cccuucauggagucauacagc

SEQ ID NO: 348 - RNA, 21 nt, Homo sapiens
ccuccaagcugagcuugcaca

SEQ ID NO: 349 - RNA, 21 nt, Homo sapiens
caacauguccacuucacugcg

SEQ ID NO: 350 - RNA, 21 nt, Homo sapiens
augcacugaaccagguucagg

SEQ ID NO: 351 - RNA, 21 nt, Homo sapiens
uucugaaugcacugaaccagg

SEQ ID NO: 352 - RNA, 21 nt, Homo sapiens
uggaauguucauuucuuuggc

SEQ ID NO: 353 - RNA, 21 nt, Homo sapiens
ugaucacccuccaagcugagc

SEQ ID NO: 354 - RNA, 21 nt, Homo sapiens
ugcccuucauggagucauaca

SEQ ID NO: 355 - RNA, 21 nt, Homo sapiens
cugaaugcacugaaccagguu

SEQ ID NO: 356 - RNA, 21 nt, Homo sapiens
cuccaagcugagcuugcacag

SEQ ID NO: 357 - RNA, 21 nt, Homo sapiens
ucacccuccaagcugagcuug

SEQ ID NO: 358 - RNA, 21 nt, Homo sapiens
agcugagcuugcacaggauuu

SEQ ID NO: 359 - RNA, 21 nt, Homo sapiens
cuguuucaauguucaaagcau

SEQ ID NO: 360 - RNA, 21 nt, Homo sapiens
uuucaacauguccacuucacu

SEQ ID NO: 361 - RNA, 21 nt, Homo sapiens
uaaucaaugacagagccaucc

SEQ ID NO: 362 - RNA, 21 nt, Homo sapiens
uuggaauguucauuucuuugg

SEQ ID NO: 363 - RNA, 21 nt, Homo sapiens
uucugaucaggaccuuaucuc

SEQ ID NO: 364 - RNA, 21 nt, Homo sapiens
uauaauaguacagggacuugc

SEQ ID NO: 365 - RNA, 21 nt, Homo sapiens
aucucaaugagagaguccucg

SEQ ID NO: 366 - RNA, 21 nt, Homo sapiens
acuucacuuccaacuccuugg

SEQ ID NO: 367 - RNA, 21 nt, Homo sapiens
ucacaucauagacuucacuuc

SEQ ID NO: 368 - RNA, 21 nt, Homo sapiens
ucauacugagcaggugucuuc

(19) United States
(12) Patent Application Publication
(10) Pub. No.: US 2007/0098812 A1
(43) Pub. Date: May 3, 2007

(54) Annexin II and uses thereof

(76) Inventors: Elena Feinstein, Rehovot (IL); Igor Mett, Rehovot (IL); Michael Shtutman, Rehovot (IL)

Correspondence Address: COOPER & DUNHAM, LLP, 1185 AVENUE OF THE AMERICAS, NEW YORK, NY, 10036, US

(21) Appl. No.: 11/528,237

(22) Filed: Sep. 26, 2006

Related U.S. Application Data
(63) Continuation-in-part of application No. PCT/IL05/00342, filed on Mar. 27, 2005.
(60) Provisional application No. 60/556,724, filed on Mar. 26, 2004.

(51) Int. Cl. A61K 48/00; A61K 31/405; C07H 21/02; A61K 33/26

(52) U.S. Cl. 424/608; 514/44; 536/23.1; 514/419

(57) ABSTRACT

The present invention relates to the field of diagnosis and treatment of neurodegenerative diseases, ischemic events, and central nervous system injury, and provides compositions and methods for alleviation or reduction of the symptoms and signs associated with damaged neuronal tissues whether resulting from tissue trauma, or from chronic or acute degenerative changes. The present invention in particular relates to the discovery that the expression of Annexin II is involved in apoptosis induced by oxidative stress, and that anti-sense Annexin II RNA and Annexin II siRNA protected the cells from this apoptosis. Thus Annexin II inhibitors prevent the damage caused by said ischemic event.

This application is a continuation-in-part of PCT International Application No. PCT/IL2005/000342, filed Mar. 27, 2005, and claims the benefit of U.S. Provisional Application No. 60/556,724, filed Mar. 26, 2004, the contents of all of which are hereby incorporated by reference into this application.

### FIELD OF THE INVENTION

The present invention relates to the field of diagnosis and treatment of neurodegenerative diseases, ischemic events, and central nervous system injury.

### BACKGROUND OF THE INVENTION

Ischemia of the Brain

Brain injury such as trauma and stroke are among the leading causes of mortality and disability in the western world.

Traumatic brain injury (TBI) is one of the most serious reasons for hospital admission and disability in modern society. Clinical experience suggests that TBI may be classified into primary damage occurring immediately after injury, and secondary damage, which occurs during several days post injury. Current therapy of TBI is either surgical or else mainly symptomatic.

Cerebrovascular diseases occur predominately in the middle and late years of life. They cause approximately 200,000 deaths in the United States each year as well as considerable neurologic disability. The incidence of stroke increases with age and affects many elderly people, a rapidly growing segment of the population. These diseases cause either ischemia-infarction or intracranial hemorrhage.

Stroke is an acute neurologic injury occurring as a result of interrupted blood supply, resulting in an insult to the brain. Most cerebrovascular diseases present as the abrupt onset of focal neurologic deficit. The deficit may remain fixed, or it may improve or progressively worsen, leading usually to irreversible neuronal damage at the core of the ischemic focus, whereas neuronal dysfunction in the penumbra may be treatable and/or reversible. Prolonged periods of ischemia result in frank tissue necrosis. Cerebral edema follows and progresses over the subsequent 2 to 4 days. If the region of the infarction is large, the edema may produce considerable mass effect with all of its attendant consequences.

Neuroprotective drugs are being developed in an effort to rescue neurons in the penumbra from dying, though as yet none has been proven efficacious.

Damage to neuronal tissue can lead to severe disability and death. The extent of the damage is primarily affected by the location and extent of the injured tissue. Endogenous cascades activated in response to the acute insult play a role in the functional outcome. Efforts to minimize, limit and/or reverse the damage have the great potential of alleviating the clinical consequences.

Annexin II

Annexin II is a tetramer, containing two heavy chains (p36, belonging to the Annexin protein family) and two light chains (p10 or p11, belonging to the S-100 protein family). Annexin binds calcium and phospolipids, and functions as a cofactor in plasminogen conversion. Trasmembrane Annexin binds plasminogen activators (both tissue and urokinase types) and activates conversion of plasminogen to plasmin up to 15 fold. (Cesarman G M, Guevara C A, Hajjar K A: An endothelial cell receptor for plasminogen/tissue plasminogen activator (t-PA). II. Annexin II-mediated enhancement of t-PA-dependent plasminogen activation. J Biol. Chem. 1994 Aug. 19; 269(33): 21198-203; Hajjar K A, Jacovina A T, Chacko J.: An endothelial cell receptor for plasminogen/tissue plasminogen activator. I. Identity with Annexin II. J Biol. Chem. 1994 Aug. 19; 269(33): 21191-7.; Kim J, Hajjar K A.: Annexin II: a plasminogen-plasminogen activator co-receptor. Front Biosci. 2002 Feb. 1; 7: d341-8.). Moreover, Annexin affects further steps of plasminogen processing and functions as a plasmin reductase (Kwon M, Caplan J F, Filipenko N R, Choi K S, Fitzpatrick S L, Zhang L, Waisman D M: Identification of Annexin II heterotetramer as a plasmin reductase. J Biol. Chem. 2002 Mar. 29; 277(13): 10903-11. Epub 2002 Jan. 7.). Purification of Annexin and its use in enzymatic reactions has been described (Choi K S, Fitzpatrick S L, Filipenko N R, Fogg D K, Kassam G, Magliocco A M, Waisman D M.: Regulation of plasmin-dependent fibrin clot lysis by Annexin II heterotetramer. J Biol. Chem. 2001 Jul. 6; 276(27): 25212-21. Epub 2001 Apr. 23). Further, Annexin II serves as profibrinolytic co-receptor for both tPA and plasminogen on the surface of endothelial cells, and facilitates the generation of plasmin.

Annexin II may contribute to the invasive potential of cancer cells through the extracellular matrix either by generation of plasmin, or by plasmin-mediated proteolytic activation of other metalloproteinases. Intracellular Annexin II has been implicated in cellular proliferation and differentiation. Annexin II secreted in the bone marrow enviroment has been implicated in osteoclastogenesis. Additionally, Annexin II has been implicated in the secretory pathway of adrenal chromaffin cells where it is found closely associated with chromaffin granules as they attach to the plasma membranes.

Additionally, Annexin II participates in membrane fusion (synergistically with arachidonic acid) during the exocytosis of lamellar bodies from alveolar epithelial type II cells (Chattopadhyay S, Sun P, Wang P, Abonyo B, Cross N L, Liu L.: Fusion of lamellar body with plasma membrane is driven by the dual action of Annexin II tetramer and arachidonic acid. J Biol. Chem. 2003 Oct. 10; 278(41): 39675-83. Epub 2003 Aug. 05.)

As a monomer, Annexin II is involved in DNA synthesis. The N-terminus of Annexin II contains Leu-rich nuclear export signal (NES) for CRM1-pathway. In the nucleus, Annexin II is phosphorylated in a cell cycle dependent manner and phosphorylation likely regulates nuclear export. Forced nuclear retention by mutation of NES leads to reduced cell proliferation (Liu J, Rothermund C A, Ayala-Sanmartin J, Vishwanatha J K.: Nuclear Annexin II negatively regulates growth of LNCaP cells and substitution of ser 11 and 25 to glu prevents nucleo-cytoplasmic shuttling of Annexin II. BMC Biochem. 2003 Sep. 9; 4(1): 10.)

Disease Relevant Patterns of Annexin II Expression

Annexin II is overexpressed in primary pancreatic cancer cells, in gastric cancer tissues and this overexpression correlates with poor prognosis. The expression of Annexin II is lost in prostate cancers (see Liu et al., above). The light chain of Annexin II binds procathepsin B (which is up-regulated in tumors) on the cell surface, and facilitates its processing (Roshy S, Sloane B F, Moin K.: Pericellular cathepsin B and malignant progression. Cancer Metastasis Rev. 2003 Jun-Sep; 22(2-3): 271-86.). In addition, the light chain of Annexin II binds and modulates the function of Hepatitis B polymerase (Choi J, Chang J S, Song M S, Ahn B Y, Park Y, Lim D S, Han Y S.: Association of hepatitis B virus polymerase with promyelocytic leukemia nuclear bodies mediated by the S100 family protein p11. Biochem Biophys Res Commun. 2003 Jun. 13; 305(4): 1049-56.)

None of the above publications disclose a role for Annexin II in connection with neurotoxic events or the diagnosis or treatment of neurodegenerative diseases such as, inter alia, stroke.

### SUMMARY OF THE INVENTION

The present invention provides compositions and methods for alleviation or reduction of the symptoms and signs associated with damaged neuronal tissues whether resulting from tissue trauma, or from chronic or acute degenerative changes.

In particular, one embodiment of the present invention provides one or more pharmaceutical compositions comprising as an active ingredient an Annexin II inhibitor further comprising a pharmaceutically acceptable diluent or carrier.

An additional embodiment provides a method for reducing damage to the central nervous system in a patient who has suffered an injury to the central nervous system, comprising administering to the patient a pharmaceutical composition in a dosage sufficient to reduce the damage. Yet another embodiment provides for the use of a Annexin II inhibitor for the preparation of a medicament for promoting or enhancing recovery in a patient who suffers from a neurodegenerative disease or an injury to the central nervous system.

An additional embodiment provides a method for identifying a chemical compound that modulates apoptosis.

Further, a process for diagnosing a neurodegenerative disease or an ischemic event in a subject is provided.

The preferred methods, materials, and examples that will now be described are illustrative only and are not intended to be limiting; materials and methods similar or equivalent to those described herein can be used in practice or testing of the invention. Other features and advantages of the invention will be apparent from the following detailed description, and from the claims.

### DETAILED DESCRIPTION OF THE INVENTION

The present invention, in some of its embodiments, provides polynucleotides, polypeptides, small molecules, compositions and methods for alleviation or reduction of the symptoms and signs associated with damaged neuronal tissues whether resulting from tissue trauma, or from acute and chronic degenerative changes. Certain aspects of the present invention provide pharmaceutical compositions which reduce or even completely diminish tissue damage or degeneration. In additional aspects, the present invention provides methods leading to functional improvement after traumatic ischemic events. These effects are achieved by administering an agent that inhibits the biological activity of Annexin II or the expression of Annexin II.

The inventors of the present invention discovered that the expression of Annexin II is involved in apoptosis induced by oxidative stress, and that anti-sense Annexin II RNA and Annexin II siRNA protected the cells from this apoptosis.

Without being bound by theory, applicants suggest that an Annexin II inhibitor can prevent neurotoxic-stress induced apoptosis of neurons that occurs during an ischemic event, and thus contribute to preventing the damage caused by said ischemic event.

The term “apoptosis” is particularly defined as execution of a built-in cell death program resulting in chromatin fragmentation into membrane-bound particles, changes in cell cytoskeleton and membrane structure and subsequent phagocytosis of apoptotic cell by other cells. Additionally, the term is understood to include ischemic disease pathologies which induce apoptosis (such as, for example, ischemic diseases which involve a decrease in the blood supply to a bodily organ, tissue or body part generally caused by constriction or obstruction of the blood vessels, as for example myocardial infarction and stroke). The term “programmed cell death” may also be used interchangeably with “apoptosis”. As used herein, it should be understood that this term should be construed more broadly as encompassing neuronal cell death, whether or not that cell death is strictly by means of the apoptotic process described above.

The term “Annexin II”, as used herein, refers to the expressed polypeptide of the Annexin II gene, also known as “Calpactin I”, “Lipocortin 2”, “Chromobindin 8”, “P36”, and “Placental anti-coagulant protein IV” (“PAP-IV”), derived from any organism, preferably man, and homologs (including the rat and murine homolog) and fragments thereof having similar biological activity. Polypeptides encoded by nucleic acid sequences which bind to the Annexin II gene under conditions of highly stringent hybridization, which are well-known in the art (for example Ausubel et al., Current Protocols in Molecular Biology, John Wiley and Sons, Baltimore, Md. (1988), updated in 1995 and 1998), are also encompassed by this term. The cDNA sequence and amino acid sequence of Annexin II are set out in FIGS. 1 and 2 respectively. Particular fragments of Annexin II include amino acids 1-50, 51-100, 101-150, 151-200, 201-250, 251-300 and 301-339 of the sequence shown in FIG. 2. Further particular fragments of Annexin II include amino acids 25-74, 75-124, 125-174, 175-224, 225-274, 275-324 and 325-339 of the sequence shown in FIG. 2.

There are at least 2 Annexin II polypeptides encoded by 3 different splice variants, for which the GeneBank references are 50845387 (variant 1) encoding a longer polypeptide and 50845385 (variant 2) and 50845389 (variant 3) that encode the same shorter polypeptide. The nucleotide sequence given in FIG. 1 is the ORF of splice variants 2 (gi-50845385) and 3 (gi-50845389). These variants differ slightly at the 5′-end of their ORF from splice variant 1 (gi-50845387), and these variants 2 and 3 differ from one another at the 5′-UTR. The corresponding polypeptide sequence to FIG. 1 has 339 amino acids; see FIG. 2. These variants and any other similar minor variants are included in the definition of Annexin II polypeptide and in the definition of the Annexin II genes encoding them.

By “biological effect of Annexin II” or “Annexin II biological activity” is meant the effect of Annexin II in apoptosis, also termed “Annexin II-induced apoptosis” herein, which may be direct or indirect, and includes, without being bound by theory, the effect of Annexin II on apoptosis induced by neurotoxic stress. The indirect effect includes, but is not limited to, Annexin II binding to or having an effect on one of several molecules, which are involved in a signal transduction cascade resulting in apoptosis.

By “Annexin II inhibitor” is meant any molecule, whether a polynucleotide, polypeptide, antibody, or small chemical compound, that prevents or reduces the biological effect of Annexin II, as recited above. An Annexin II inhibitor may also be an inhibitor of the Annexin II promoter or of Annexin II transcription/translation such as an antisense RNA molecule, siRNA, dominant negative peptide, ribozyme, inter alia.

One aspect of the present invention provides for a pharmaceutical composition comprising as an active ingredient a Annexin II inhibitor in a therapeutically effective amount, which may be a small chemical compound, such as sodium nitroprusside (Liu et al., Eur. J. Biochem. 269, 4277-4286 (2002)), or the tyrosine kinase inhibitor Tyrphostin AG1024 which inhibits AnnexinII secretion (Zhao et al., JBC 278, 6: 4205-4215 (2003)); a polynucleotide, such as an antisense polynucleotide comprising consecutive nucleotides having a sequence which is an antisense sequence to the sequence set forth in FIG. 1 (SEQ ID NO:1), optionally having one of the sequences set forth in FIG. 3 (SEQ ID NO:3 or SEQ ID NO:4), or a polynucleotide which is a sense polynucleotide comprising consecutive nucleotides having a sequence which is a sense sequence to the sequence set forth in FIG. 1 (SEQ ID NO:1), and which encodes a dominant negative peptide to said sequence, optionally having one of the sequences set forth in FIG. 4 (SEQ ID NO:5 or SEQ ID NO:6) or a polynucleotide that functions as silencing RNA (siRNA), optionally having one of the sequence set forth in Tables 1-3, particularly Table 1; a vector comprising any of these polynucleotides; a polypeptide, such as a dominant negative peptide, for example, the peptide encoded by SEQ ID NO:5 or SEQ ID NO:6, peptide #41 of PCT patent application publication No. WO 200404/1844 which was found to bind annexin II, S-nitrosogluthathione (GSNO; Liu et al., Eur. J. Biochem. 269, 4277-4286 (2002)) or an antibody, optionally a polyclonal or a monoclonal antibody, such as the anti-Annexin II antibody disclosed in Pietropaolo & Compton: Direct interaction between human cytomegalovirus glycoprotein B and cellular Annexin II. J Virol 1997, 71: 9803-9807, inter alia. The pharmaceutical composition may further contain a diluent or carrier.

Another aspect of the present invention concerns a method for treating a patient suffering from a neurodegenerative disease and/or a central nervous system (CNS) disorder, comprising administering to the patient a therapeutically effective amount of an Annexin II inhibitor, as as to thereby treat the patient. Administration may be periodical. The Annexin II inhibitor may be a small chemical compound, such as sodium nitroprusside (Liu et al., Eur. J. Biochem. 269, 4277-4286 (2002)), or the tyrosine kinase inhibitor Tyrphostin AG1024 which inhibits AnnexinII secretion (Zhao et al., JBC 278, 6: 4205-4215 (2003)); a polynucleotide, such as an antisense polynucleotide comprising consecutive nucleotides having a sequence which is an antisense sequence to the sequence set forth in FIG. 1 (SEQ ID NO:1), optionally having one of the sequences set forth in FIG. 3 (SEQ ID NO:3 or SEQ ID NO:4), or a polynucleotide which is a sense polynucleotide comprising consecutive nucleotides having a sequence which is a sense sequence to the sequence set forth in FIG. 1 (SEQ ID NO:1), and which encodes a dominant negative peptide to said sequence, optionally having one of the sequences set forth in FIG. 4 (SEQ ID NO:5 or SEQ ID NO:6) or a polynucleotide that functions as silencing RNA (siRNA), optionally having one of the sequence set forth in Tables 1-3, particularly in Table 1 SEQ ID No.'s z-z; a vector comprising any of these polynucleotides; a polypeptide, such as a dominant negative peptide, for example, the peptide encoded by SEQ ID NO:5 or SEQ ID NO:6, peptide #41 of PCT patent application publication No. WO 200404/1844 which was found to bind annexin II, S-nitrosogluthathione (GSNO; Liu et al., Eur. J. Biochem. 269, 4277-4286 (2002)) or an antibody, optionally a polyclonal or a monoclonal antibody such as the anti-Annexin II antibody disclosed in Pietropaolo & Compton: Direct interaction between human cytomegalovirus glycoprotein B and cellular Annexin II. J Virol 1997, 71: 9803-9807, inter alia.

Further provided in this aspect is the use of a therapeutically effective amount of an Annexin II inhibitor, such as any of the inhibitors detailed above, for the preparation of a medicament for promoting or enhancing recovery in a patient suffering from a neurodegenerative disease or an injury to the central nervous system.

Additionally, the present invention provides a method of regulating a pathology or disease (as recited above) in a patient in need of such treatment by administering to a patient a therapeutically effective dose of at least one inhibitor e.g. at least one antisense (AS) oligonucleotide or at least one siRNA against the nucleic acid sequences or a dominant negative peptide directed against the Annexin II sequences or Annexin II proteins or an antibody directed against the Annexin II polypeptide, or any of the inhibitors described above.

The terms “chemical compound”, “small molecule”, “chemical molecule” “small chemical molecule” and “small chemical compound” are used interchangeably herein and are understood to refer to chemical moieties of any particular type which may be synthetically produced or obtained from natural sources and typically have a molecular weight of less than 2000 daltons, more preferably less than 1000 daltons or even less than 600 daltons.

The term “polynucleotide” refers to any molecule composed of DNA nucleotides, RNA nucleotides or a combination of both types, i.e. that comprises two or more of the bases guanidine, cytosine, thymidine, adenine, uracil or inosine, inter alia. A polynucleotide may include natural nucleotides, chemically modified nucleotides and synthetic nucleotides, or chemical analogs thereof. The term includes “oligonucleotides” and encompasses “nucleic acids”.

A polynucleotide generally has from about 75 to 10,000 nucleotides, optionally from about 100 to 3,500 nucleotides. An oligonucleotide refers generally to a chain of nucleotides extending from 2-75 nucleotides.

By the term “antisense” (AS) or “antisense fragment” is meant a polynucleotide fragment having inhibitory antisense activity, said activity causing a decrease in the expression of the endogenous genomic copy of the corresponding gene (in this case Annexin II). An Annexin II AS polynucleotide is a polynucleotide which comprises consecutive nucleotides having a sequence of sufficient length and homology to a sequence present within the sequence of the Annexin II gene set forth in SEQ ID NO:1 to permit hybridization of the AS to the gene. The sequence of the AS is designed to complement a target mRNA of interest and form an RNA:AS duplex. This duplex formation can prevent processing, splicing, transport or translation of the relevant mRNA. Moreover, certain AS nucleotide sequences can elicit cellular RNase H activity when hybridized with their target mRNA, resulting in mRNA degradation (Calabretta et al, 1996: Antisense strategies in the treatment of leukemias. Semin Oncol. 23(1):78-87). In that case, RNase H will cleave the RNA component of the duplex and can potentially release the AS to further hybridize with additional molecules of the target RNA. An additional mode of action results from the interaction of AS with genomic DNA to form a triple helix which can be transcriptionally inactive. Particular AS fragments are the AS of the DNA encoding the particular fragments of Annexin II described herein. The AS fragment of the present invention optionally has the sequence depicted in FIG. 3 or a homologous sequence thereof. Particular AS fragments are the AS of the DNA encoding the particular fragments of Annexin II described above. For delivery of AS fragments see Example 12.

Many reviews have covered the main aspects of antisense (AS) technology and its therapeutic potential (Wright & Anazodo, 1995. Antisense Molecules and Their Potential For The Treatment Of Cancer and AIDS. Cancer J. 8:185-189.). There are reviews on the chemical (Crooke, 1995. Progress in antisense therapeutics, Hematol. Pathol. 2:59; Uhlmann and Peyman, 1990. Antisense Oligonucleolides: A New Therapeutic Principle. Chem Rev 90(4):543-584.), cellular (Wagner, 1994. Gene inhibition using antisense oligodeoxynucleotides. Nature 372:333.) and therapeutic (Hanania, et al 1995. Recent advances in the application of gene therapy to human disease. Am. J. Med. 99:537.; Scanlon et al., 1995. Oligonucleotides-mediated modulation of mammalian gene expression. FASEB J. 9:1288.; Gewirtz, 1993. Oligodeoxynucleotide-based therapeutics for human leukemias, Stem Cells Dayt. 11:96.) aspects of this technology.

Antisense intervention in the expression of specific genes can be achieved by the use of synthetic AS oligonucleotide sequences (see Lefebvre-d'Hellencourt et al, 1995. Immunomodulation by cytokine antisense oligonucleotides. Eur. Cytokine Netw. 6:7.; Agrawal, 1996. Antisense oligonucleotides: towards clinical trials, TIBTECH, 14:376.; Lev-Lehman et al., 1997. Antisense Oligomers in vitro and in vivo. In Antisense Therapeutics, A. Cohen and S. Smicek, eds (Plenum Press, New York)). AS oligonucleotide sequences are designed to complement a target mRNA of interest and form an RNA:AS duplex. This duplex formation can prevent processing, splicing, transport or translation of the relevant mRNA. Moreover, certain AS nucleotide sequences can elicit cellular RNase H activity when hybridized with their target mRNA, resulting in mRNA degradation (Calabretta, et al, 1996. Antisense strategies in the treatment of leukemias. Semin. Oncol. 23:78.). In that case, RNase H will cleave the RNA component of the duplex and can potentially release the AS to further hybridize with additional molecules of the target RNA. An additional mode of action results from the interaction of AS with genomic DNA to form a triple helix which may be transcriptionally inactive.

The sequence target segment for the antisense oligonucleotide is selected such that the sequence exhibits suitable energy related characteristics important for oligonucleotide duplex formation with their complementary templates, and shows a low potential for self-dimerization or self-complementation (Anazodo et al., 1996). For example, the computer program OLIGO (Primer Analysis Software, Version 3.4), can be used to determine antisense sequence melting temperature, free energy properties, and to estimate potential self-dimer formation and self-complimentary properties. The program allows the determination of a qualitative estimation of these two parameters (potential self-dimer formation and self-complimentary) and provides an indication of “no potential” or “some potential” or “essentially complete potential”. Using this program target segments are generally selected that have estimates of no potential in these parameters. However, segments can be used that have “some potential” in one of the categories. A balance of the parameters is used in the selection as is known in the art. Further, the oligonucleotides are also selected as needed so that analogue substitution do not substantially affect function.

Phosphorothioate antisense oligonucleotides do not normally show significant toxicity at concentrations that are effective and exhibit sufficient pharmacodynamic half-lives in animals (Agrawal, 1996. Antisense oligonucleotides: towards clinical trials, TIBTECH, 14:376.) and are nuclease resistant. Antisense induced loss-of-function phenotypes related with cellular development were shown for the glial fibrillary acidic protein (GFAP), for the establishment of tectal plate formation in chick (Galileo et al., 1991. J. Cell. Biol., 112:1285.) and for the N-myc protein, responsible for the maintenance of cellular heterogeneity in neuroectodermal cultures (ephithelial vs. neuroblastic cells, which differ in their colony forming abilities, tumorigenicity and adherence) (Rosolen et al., 1990. Cancer Res. 50:6316.; Whitesell et al., 1991. Episome-generated N-myc antisense RNA restricts the differentiation potential of primitive neuroectodermal cell lines. Mol. Cell. Biol. 11:1360.). Antisense oligonucleotide inhibition of basic fibroblast growth factor (bFgF), having mitogenic and angiogenic properties, suppressed 80% of growth in glioma cells (Morrison, 1991. Suppression of basic fibroblast growth factor expression by antisense oligonucleotides inhibits the growth of transformed human astrocytes. J. Biol. Chem. 266:728.) in a saturable and specific manner. Being hydrophobic, antisense oligonucleotides interact well with phospholipid membranes (Akhter et al, 1991. Interactions of antisense DNA oligonucleotide analogs with phospholipid membranes (liposomes) Nuc. Res. 19:5551-5559.). Following their interaction with the cellular plasma membrane, they are actively (or passively) transported into living cells (Loke et al, 1989. Characterization of oligonucleotide transport into living cells. PNAS USA 86:3474.), in a saturable mechanism predicted to involve specific receptors (Yakubov et al, 1989. PNAS USA 86:6454.).

A “ribozyme” is an RNA molecule that possesses RNA catalytic ability (see Cech for review) and cleaves a specific site in a target RNA.

In accordance with the present invention, ribozymes which cleave Annexin II mRNA may be utilized as Annexin II inhibitors. This may be necessary in cases where antisense therapy is limited by stoichiometric considerations (Sarver et al., 1990, Gene Regulation and Aids, pp. 305-325). Ribozymes can then be used that will target the Annexin II sequence. The number of RNA molecules that are cleaved by a ribozyme is greater than the number predicted by stochiochemistry (Hampel and Tritz, 1989; Uhlenbeck, 1987).

Ribozymes catalyze the phosphodiester bond cleavage of RNA. Several ribozyme structural families have been identified including Group I introns, RNase P, the hepatitis delta virus ribozyme, hammerhead ribozymes and the hairpin ribozyme originally derived from the negative strand of the tobacco ringspot virus satellite RNA (sTRSV) (Sullivan, 1994; U.S. Pat. No. 5,225,347, columns 4-5). The latter two families are derived from viroids and virusoids, in which the ribozyme is believed to separate monomers from oligomers created during rolling circle replication (Symons, 1989 and 1992). Hammerhead and hairpin ribozyme motifs are most commonly adapted for trans-cleavage of mRNAs for gene therapy (Sullivan, 1994). The ribozyme type utilized in the present invention is selected as is Known in the art. Hairpin ribozymes are now in clinical trial and are the preferred type. In general the ribozyme is from 30-100 nucleotides in length. Delivery of ribozymes is similar to that of AS fragments and/or siRNA molecules.

By siRNA is meant an RNA molecule which decreases or silences (prevents) the expression of a gene/mRNA of its endogenous cellular counterpart. The term is understood to encompass “RNA interference” (RNAi). RNA interference (RNAi) refers to the process of sequence-specific post transcriptional gene silencing in mammals mediated by small interfering RNAs (siRNAs) (Fire et al, 1998, Nature 391, 806). The corresponding process in plants is commonly referred to as specific post transcriptional gene silencing or RNA silencing and is also referred to as quelling in fungi. The RNA interference response may feature an endonuclease complex containing an siRNA, commonly referred to as an RNA-induced silencing complex (RISC), which mediates cleavage of single-stranded RNA having sequence complementary to the antisense strand of the siRNA duplex. Cleavage of the target RNA may take place in the middle of the region complementary to the antisense strand of the siRNA duplex (Elbashir et al 2001, Genes Dev., 15, 188). For recent information on these terms and proposed mechanisms, see Bernstein E., Denli A M., Hannon G J: The rest is silence. RNA. 2001 November; 7(11):1509-21; and Nishikura K.: A short primer on RNAi: RNA-directed RNA polymerase acts as a key catalyst. Cell. 2001 Nov. 16; 107(4):415-8. Examples of the nucleotide sequence of siRNA molecules which may be used in the present invention are given in Tables 1-3, and the chemical modifications used are described in PCT patent application publication No. WO2004035615 (atugen).

During recent years, RNAi has emerged as one of the most efficient methods for inactivation of genes (Nature Reviews, 2002, v.3, p. 737-47; Nature, 2002, v.418,p. 244-51). As a method, it is based on the ability of dsRNA species to enter a specific protein complex, where it is then targeted to the complementary cellular RNA and specifically degrades it. In more detail, dsRNAs are digested into short (17-29 bp) inhibitory RNAs (siRNAs) by type III RNAses (DICER, Drosha, etc) (Nature, 2001, v.409, p. 363-6; Nature, 2003, 425, p. 415-9). These fragments and complementary mRNA are recognized by specific RISC protein complex. The whole process is culminated by endonuclease cleavage of target mRNA (Nature Reviews, 2002, v.3, p. 737-47; Curr Opin Mol Ther. 2003 June; 5(3):217-24).

For disclosure on how to prepare siRNA to known genes see for example Chalk A M, Wahlestedt C, Sonnhammer E L. Improved and automated prediction of effective siRNA Biochem. Biophys. Res. Commun. 2004 Jun. 18; 319(1):264-74; Sioud M, Leirdal M., Potential design rules and enzymatic synthesis of siRNAs, Methods Mol. Biol. 2004; 252:457-69; Levenkova N, Gu Q, Rux J J.: Gene specific siRNA selector Bioinformatics. 2004 Feb. 12; 20(3):430-2. and Ui-Tei K, Naito Y, Takahashi F, Haraguchi T, Ohki-Hamazaki H, Juni A, Ueda R, Saigo K., Guidelines for the selection of highly effective siRNA sequences for mammalian and chick RNA interference Nucleic Acids Res. 2004 Feb. 9; 32(3):936-48. See also Liu Y, Braasch D A, Nulf C J, Corey D R. Efficient and isoform-selective inhibition of cellular gene expression by peptide nucleic acids Biochemistry, 2004 Feb. 24; 43(7):1921-7. See also PCT publications WO 2004/015107 (Atugen) and WO 02/44321 (Tuschl et al), and also Chiu Y L, Rana T M. siRNA function in RNAi: a chemical modification analysis, RNA 2003 September; 9(9):1034-48 and U.S. Pat. Nos. 5,898,031 and 6,107,094 (Crooke) for production of modified/more stable siRNAs.

DNA-based vectors capable of generating siRNA within cells have been developed. The method generally involves transcription of short hairpin RNAs that are efficiently processed to form siRNAs within cells. Paddison et al. PNAS 2002, 99:1443-1448; Paddison et al. Genes & Dev 2002, 16:948-958; Sui et al. PNAS 2002, 8:5515-5520; and Brummelkamp et al. Science 2002, 296:550-553. These reports describe methods to generate siRNAs capable of specifically targeting numerous endogenously and exogenously expressed genes.

For delivery of siRNAs, see, for example, Shen et al (FEBS letters 539: 111-114 (2003)), Xia et al., Nature Biotechnology 20: 1006-1010 (2002), Reich et al., Molecular Vision 9: 210-216 (2003), Sorensen et al. (J. Mol. Biol. 327: 761-766 (2003), Lewis et al., Nature Genetics 32: 107-108 (2002) and Simeoni et al., Nucleic Acids Research 31, 11: 2717-2724 (2003). siRNA has recently been successfully used for inhibition in primates; for further details see Tolentino et al., Retina 24(1) February 2004 pp 132-138.

siRNAs of the Present Invention

General Specifications of siRNAs of the Present Invention

Generally, the siRNAs used in the present invention comprise a ribonucleic acid comprising a double stranded structure, whereby the double-stranded structure comprises a first strand and a second strand, whereby the first strand comprises a first stretch of contiguous nucleotides and whereby said first stretch is at least partially complementary to a target nucleic acid, and the second strand comprises a second stretch of contiguous nucleotides and whereby said second stretch is at least partially identical to a target nucleic acid, whereby said first strand and/or said second strand comprises a plurality of groups of modified nucleotides having a modification at the 2′-position whereby within the strand each group of modified nucleotides is flanked on one or both sides by a flanking group of nucleotides whereby the flanking nucleotides forming the flanking group of nucleotides is either an unmodified nucleotide or a nucleotide having a modification different from the modification of the modified nucleotides. Further, said first strand and/or said second strand may comprise said plurality of modified nucleotides and may comprises said plurality of groups of modified nucleotides.

The group of modified nucleotides and/or the group of flanking nucleotides may comprise a number of nucleotides whereby the number is selected from the group comprising one nucleotide to 10 nucleotides. In connection with any ranges specified herein it is to be understood that each range discloses any individual integer between the respective figures used to define the range including said two figures defining said range. In the present case the group thus comprises one nucleotide, two nucleotides, three nucleotides, four nucleotides, five nucleotides, six nucleotides, seven nucleotides, eight nucleotides, nine nucleotides and ten nucleotides.

The pattern of modified nucleotides of said first strand may be the same as the pattern of modified nucleotides of said second strand, and may align with the pattern of said second strand. Additionally, the pattern of said first strand may be shifted by one or more nucleotides relative to the pattern of the second strand.

The modifications discussed above may be selected from the group comprising amino, fluoro, methoxy, alkoxy and alkyl.

The double stranded structure of the siRNA may be blunt ended, on one or both sides. More specifically, the double stranded structure may be blunt ended on the double stranded structure's side which is defined by the S″-end of the first strand and the 3′-end of the second strand, or the double stranded structure may be blunt ended on the double stranded structure's side which is defined by at the 3′-end of the first strand and the 5′-end of the second strand.

Additionally, at least one of the two strands may have an overhang of at least one nucleotide at the 5′-end; the overhang may consist of at least one deoxyribonucleotide. At least one of the strands may also optionally have an overhang of at least one nucleotide at the 3′-end.

The length of the double-stranded structure of the siRNA is typically from about 17 to 21 and more preferably 18 or 19 bases. Further, the length of said first strand and/or the length of said second strand may independently from each other be selected from the group comprising the ranges of from about 15 to about 23 bases, 17 to 21 bases and 18 or 19 bases.

Additionally, the complementarily between said first strand and the target nucleic acid may be perfect, or the duplex formed between the first strand and the target nucleic acid may comprise at least 15 nucleotides wherein there is one mismatch or two mismatches between said first strand and the target nucleic acid forming said double-stranded structure.

In some cases both the first strand and the second strand each comprise at least one group of modified nucleotides and at least one flanking group of nucleotides, whereby each group of modified nucleotides comprises at least one nucleotide and whereby each flanking group of nucleotides comprising at least one nucleotide with each group of modified nucleotides of the first strand being aligned with a flanking group of nucleotides on the second strand, whereby the most terminal S' nucleotide of the first strand is a nucleotide of the group of modified nucleotides, and the most terminal 3′ nucleotide of the second strand is a nucleotide of the flanking group of nucleotides. Each group of modified nucleotides may consist of a single nucleotide and/or each flanking group of nucleotides may consist of a single nucleotide.

Additionally, it is possible that on the first strand the nucleotide forming the flanking group of nucleotides is an unmodified nucleotide which is arranged in a 3′ direction relative to the nucleotide forming the group of modified nucleotides, and on the second strand the nucleotide forming the group of modified nucleotides is a modified nucleotide which is arranged in 5′ direction relative to the nucleotide forming the flanking group of nucleotides.

Further the first strand of the siRNA may comprise eight to twelve, preferably nine to eleven, groups of modified nucleotides, and the second strand may comprise seven to eleven, preferably eight to ten, groups of modified nucleotides.

The first strand and the second strand may be linked by a loop structure, which may be comprised of a non-nucleic acid polymer such as, inter alia, polyethylene glycol. Alternatively, the loop structure may be comprised of a nucleic acid.

Further, the 5′-terminus of the first strand of the siRNA may be linked to the 3′-terminus of the second strand, or the 3′-end of the first strand may be linked to the 5′-terminus of the second strand.

Particular Specifications of siRNAs of the Present Invention

The present invention provides double-stranded oligoribonucleotides (siRNAs), which down-regulate the expression of Annexin II gene. An siRNA of the invention is a duplex oligoribonucleotide in which the sense strand is derived from the mRNA sequence of Annexin II gene, and the antisense strand is complementary to the sense strand. In general, some deviation from the target mRNA sequence is tolerated without compromising the siRNA activity (see e.g. Czauderna et al 2003 Nucleic Acids Research 31(11), 2705-2716). An siRNA of the invention inhibits gene expression on a post-transcriptional level with or without destroying the mRNA. Without being bound by theory, siRNA may target the mRNA for specific cleavage and degradation and/or may inhibit translation from the targeted message.

More particularly, the invention provides a compound having the structure (structure A):
5′ (N)ₓ-Z 3′ (antisense strand)
3′ Z′-(N′)_{y} 5′ (sense strand)
wherein each N and N′ is a ribonucleotide which may be modified or unmodified in its sugar residue and (N)ₓ and (N′)_{y} is oligomer in which each consecutive N or N′ is joined to the next N or N′ by a covalent bond;
wherein each of x and y is an integer between 19 and 40;
wherein each of Z and Z′ may be present or absent, but if present is dTdT and is covalently attached at the 3′ terminus of the strand in which it is present;
and wherein the sequence of (N)ₓ comprises an antisense sequence to cDNA of Annexin II.

In particular, the invention provides the above compound wherein the sequence of (N)ₓ comprises one or more of the antisense sequences present in Tables 1, 2 and 3.

It will be readily understood by those skilled in the art that the compounds of the present invention consist of a plurality of nucleotides which are linked through covalent linkages. Each such covalent linkage may be a phosphodiester linkage, a phosphothioate linkage, or a combination of both, along the length of the nucleotide sequence of the individual strand. Other possible backbone modifications are described inter alia in U.S. Pat. Nos. 5,587,361; 6,242,589; 6,277,967; 6,326,358; 5,399,676; 5,489,677; and 5,596,086.

In particular embodiments, x and y are preferably an integer between about 19 to about 27, most preferably from about 19 to about 23. In a particular embodiment of the compound of the invention, x may be equal to y (viz., x=y) and in preferred embodiments x=y=19 or x=y=21. In a particularly preferred embodiment x=y=19.

In one embodiment of the compound of the invention, Z and Z′ are both absent; in another embodiment one of Z or Z' is present.

In one embodiment of the compound of the invention, all of the ribonucleotides of the compound are ummodified in their sugar residues.

In some embodiments of the compound of the invention, at least one ribonucleotide is modified in its sugar residue, preferably a modification at the 2′ position. The modification at the 2′ position results in the presence of a moiety which is preferably selected from the group comprising amino, fluoro, methoxy, alkoxy and alkyl groups. In a presently most preferred embodiment the moiety at the 2′ position is methoxy (2′-O-methyl).

In some embodiments of the invention, alternating ribonucleotides are modified in both the antisense and the sense strands of the compound.

In particularly preferred embodiments of the invention, the antisense strand is phophorylated at the 5′terminus, and may or may not be phophorylated at the 3′terminus; and the sense strand may or may not be phophorylated at the 5′terminus and at the 3′terminus.

In another embodiment of the compound of the invention, the ribonucleotides at the 5′ and 3′ termini of the antisense strand are modified in their sugar residues, and the ribonucleotides at the 5′ and 3′ termini of the sense strand are unmodified in their sugar residues.

The invention further provides a vector capable of expressing any of the aforementioned oligoribonucleotides in unmodified form in a cell after which appropriate modification may be made.

The invention also provides a composition comprising one or more of the compounds of the invention in a carrier, preferably a pharmaceutically acceptable carrier. This composition may comprise a mixture of two or more different siRNAs for the same gene.

Another compound of the invention comprises the above compound of the invention (structure A) covalently or non-covalently bound to one or more compounds of the invention (structure A). This compound may be delivered in a carrier, preferably a pharmaceutically acceptable carrier, and may be processed intracellularly by endogenous cellular complexes to produce one or more siRNAs of the invention.

The invention also provides a composition comprising a carrier and one or more of the compounds of the invention in an amount effective to down-regulate expression in a cell of a human Annexin II, which compound comprises a sequence substantially complementary to the sequence of (N)ₓ.

The invention also provides a method of down-regulating the expression of a human Annexin II gene by at least 50% as compared to a control comprising contacting an mRNA transcript of the gene with one or more of the compounds of the invention.

In one embodiment the compound is down-regulating Annexin II polypeptide, whereby the down-regulation of Annexin II is selected from the group comprising down-regulation of Annexin II function (which may be examined by an enzymatic assay or a binding assay with a known interactor of the native gene/polypeptide, inter alia), down-regulation of Annexin II protein (which may be examined by Western blotting, ELISA or immuno-precipitation, inter alia) and down-regulation of Annexin II mRNA expression (which may be examined by Northern blotting, quantitative RT-PCR, in-situ hybridisation or microarray hybridisation, inter alia).

The invention also provides a method of treating a patient suffering from a neurodegenerative disease and/or an injury to the central nervous system, comprising administering to the patient a composition of the invention in a therapeutically effective dose so as to thereby treat the patient.

The invention also provides a use of a therapeutically effective dose of one or more compounds of the invention for the preparation of a composition for promoting recovery in a patient suffering from a neurodegenerative disease and/or a pathology of the central nervous system.

The term “treatment” as used herein refers to administration of a therapeutic substance effective to ameliorate symptoms associated with a disease, to lessen the severity or cure the disease, or to prevent the disease from occurring.

The compound may have homologs wherein up to two of the ribonucleotides in each terminal region a base is altered; the terminal region refers to the four terminal ribonucleotides e.g. refers to bases 1-4 and/or 16-19 in a 19-mer sequence and to bases 1-4 and/or 18-21 in a 21-mer sequence.

The preferred oligonucleotides of the invention are the oligonucleotides listed in Tables 1, 2 and 3, preferably the oligonucleotides listed in Table 1 and/or the oligonucleotides targeting human cDNA. The most preferred oligonucleotides of the invention are the oligonucleotides having inhibitory activity as demonstrated in Table 1, preferably oligonucleotides targeting humanAnnexin II cDNA.

The presently most preferred compound of the invention is a blunt-ended 1 g-mer oligonucleotide, i.e. x=y=19 and Z and Z′ are both absent; the oligonucleotide is phophorylated at the 5′position of the antisense strand and at the 3′ position of the sense strand wherein alternating ribonucleotides are modified at the 2′ position in both the antisense and the sense strands, wherein the moiety at the 2′ position is methoxy (2′-O-methyl) and wherein the ribonucleotides at the 5′ and 3′ termini of the antisense strand are modified in their sugar residues, and the ribonucleotides at the 5′ and 3′ termini of the sense strand are unmodified in their sugar residues. The presently most preferred such compound is siRNA No. 5 of Table 1. The antisense strand of this compound has SEQ ID NO:12 and the sense strand has SEQ ID NO:7. Other preferred compounds are the other siRNAs of Table 1.

In one aspect of the invention the oligonucleotide comprises a double-stranded structure, whereby such double-stranded structure comprises
a first strand and a second strand, whereby
the first strand comprises a first stretch of contiguous nucleotides and the second strand comprises a second stretch of contiguous nucleotides, whereby
the first stretch is either complementary or identical to a nucleic acid sequence coding for Annexin II and whereby the second stretch is either identical or complementary to a nucleic acid sequence coding for Annexin II.

In an embodiment the first stretch and/or the second stretch comprises from about 14 to 40 nucleotides, preferably about 18 to 30 nucleotides, more preferably from about 19 to 27 nucleotides and most preferably from about 19 to 23 nucleotides, in particular from about 19 to 21 nucleotides. In such an embodiment the oligonucleotide may be from 17-40 nucleotides in length.

Additionally, further nucleic acids according to the present invention comprise at least 14 contiguous nucleotides of any one of the SEQ. ID. NO. 7-368, and more preferably 14 contiguous nucleotide base pairs at any end of the double-stranded structure comprised of the first stretch and second stretch as described above.

The present invention also provides for a process of preparing a pharmaceutical composition, which comprises:

obtaining at least one double stranded siRNA compound of the invention; and

admixing said compound with a pharmaceutically acceptable carrier.

The present invention also provides for a process of preparing a pharmaceutical composition, which comprises admixing a compound of the present invention with a pharmaceutically acceptable carrier. The present invention also relates analogously to medicaments and methods for use in veterinary practice for the treatment and care of animals and especially for use in the treatment and care of mammals in a preferred embodiment, the compound used in the preparation of a pharmaceutical composition is admixed with a carrier in a pharmaceutically effective dose. In a particular embodiment the compound of the present invention is conjugated to a steroid or to a lipid or to another suitable molecule e.g. to cholesterol.

The compounds of the present invention can be delivered either directly or with viral or non-viral vectors. When delivered directly the sequences are generally rendered nuclease resistant. Alternatively the sequences can be incorporated into expression cassettes or constructs such that the sequence is expressed in the cell as discussed herein below. Generally the construct contains the proper regulatory sequence or promoter to allow the sequence to be expressed in the targeted cell. Vectors optionally used for delivery of the compounds of the present invention are commercially available, and may be modified for the purpose of delivery of the compounds of the present invention by methods known to one of skill in the art.

It is also envisaged that a long oligonucleotide (typically 25-500 nucleotides in length) comprising one or more stem and loop structures, where stem regions comprise the sequences of the oligonucleotides of the invention, may be delivered in a carrier, preferably a pharmaceutically acceptable carrier, and may be processed intracellularly by endogenous cellular complexes (e.g. by DROSHA and DICER as described above) to produce one or more smaller double stranded oligonucleotides (siRNAs) which are oligonucleotides of the invention. This oligonucleotide can be termed a tandem shRNA construct. It is envisaged that this long oligonucleotide is a single stranded oligonucleotide comprising one or more stem and loop structures, wherein each stem region comprises a portion of a sense and corresponding antisense siRNA sequence of the Annexin II gene, preferably a sequence present in tables 1-3.

In particular, the siRNA used in the present invention are an oligoribonucleotide wherein one strand comprises consecutive nucleotides having, from 5′ to 3′, the sequence set forth in SEQ ID NOS: 3-52 or in SEQ ID NOS: 103-174 or in SEQ ID NOS: 247-295 (which are sense strands) wherein a plurality of the bases may be modified, preferably by a 2-O-methyl modification, or a homolog thereof wherein in up to 2 of the nucleotides in each terminal region a base is altered.

The terminal region of the oligonucleotide refers to bases 1-4 and/or 16-19 in the 19-mer sequences (Tables 1 and 2 below) and to bases 1-4 and/or 18-21 in the 21-mer sequences (Table 3 below).

Additionally, the siRNAs used in the present invention are oligoribonucleotides wherein one strand comprises consecutive nucleotides having, from 5′ to 3′, the sequence set forth SEQ ID NOS: 12-16 or SEQ ID NOS: 119-220 or SEQ ID NOS: 295-368 (antisense strands) or a homolog thereof wherein in up to 2 of the nucleotides in each terminal region a base is altered.

Thus, in particular aspects the oligonucleotide comprises a double-stranded structure, whereby such double-stranded structure comprises a first strand and a second strand, whereby the first strand comprises a first stretch of contiguous nucleotides and the second strand comprises a second stretch of contiguous nucleotides, whereby the first stretch is either complementary or identical to a nucleic acid sequence coding for gene Annexin II and whereby the second stretch is either identical or complementary to a nucleic acid sequence coding for Annexin II. Said first stretch comprises at least 14 nucleotides, preferably at least 18 nucleotides and even more preferably 19 nucleotides or even at least 21 nucleotides. In an embodiment the first stretch comprises from about 14 to 40 nucleotides, preferably about 18 to 30 nucleotides, more preferably from about 19 to 27 nucleotides and most preferably from about 19 to 23 nucleotides. In an embodiment the second stretch comprises from about 14 to 40 nucleotides, preferably about 18 to 30 nucleotides, more preferably from about 19 to 27 nucleotides and most preferably from about 19 to 23 nucleotides or even about 19 to 21 nucleotides. In an embodiment the first nucleotide of the first stretch corresponds to a nucleotide of the nucleic acid sequence coding for Annexin II, whereby the last nucleotide of the first stretch corresponds to a nucleotide of the nucleic acid sequence coding for Annexin II. In an embodiment the first stretch comprises a sequence of at least 14 contiguous nucleotides of an oligonucleotide, whereby such oligonucleotide is selected from the group comprising SEQ. ID. Nos_{—}7-368 preferably from the group comprising the oligoribonucleotides of having the sequence of any of the serial numbers 1-5 in Table 1, 100-107 in Table 2, and 174-181 in Table 3. Additionally specifications of the siRNA molecules used in the present invention may provide an oligoribonucleotide wherein the dinucleotide dTdT is covalently attached to the 3′ terminus, and/or in at least one nucleotide a sugar residue is modified, possibly with a modification comprising a 2′-O-methyl modification. Further, the 2′ OH group may be replaced by a group or moiety selected from the group comprising —H—OCH₃, —OCH₂CH₃, —OCH₂CH₂ CH₃, —NH₂, and —F.

Additionally, the siRNAs used in the present invention may be an oligoribonucleotide wherein in alternating nucleotides modified sugars are located in both strands. Particularly, the oligoribonucleotide may comprise one of the sense strands wherein the sugar is unmodified in the terminal 5′ and 3′ nucleotides, or one of the antisense strands wherein the sugar is modified in the terminal 5′ and 3′ nucleotides.

Additionally, further nucleic acids to be used in the present invention comprise at least 14 contiguous nucleotides of any one of the SEQ. ID. NO. 7 to 368, and more preferably 14 contiguous nucleotide base pairs at any end of the double-stranded structure comprised of the first stretch and second stretch as described above. It will be understood by one skilled in the art that given the potential length of the nucleic acid according to the present invention and particularly of the individual stretches forming such nucleic acid according to the present invention, some shifts relative to the coding sequence of the Annexin II gene as detailed in SEQ ID NO: 1 to each side is possible, whereby such shifts can be up to 1, 2, 3, 4, 5 and 6 nucleotides in both directions, and whereby the thus generated double-stranded nucleic acid molecules shall also be within the present invention.

siRNA for Annexin II can be made using methods known in the art as described herein, based on the known sequence of Annexin II (SEQ ID NO:1), and can be made stable by various modifications as described above. For further information, see Example 4.

Further, in relation to the methods of the present invention as described herein, additional inhibitory RNA molecules of the present invention, which may be used with the methods of the present invention include single stranded oligoribonucleotides preferably comprising stretches of at least 7-14 consecutive nucleotides present in the sequences detailed in Tables 1-3 (19mers and 21 mers), said oligoribonucleotides being capable of forming and/or said oligoribonucleotides comprising double stranded regions in particular conformations that are recognized by intracellular complexes, leading to the degradation of said oligoribonucleotides into smaller RNA molecules that are capable of exerting inhibition of Annexin II, and DNA molecules encoding such RNA molecules.

Any molecules, such as, for example, antisense DNA molecules which comprise the siRNA sequences disclosed herein (with the appropriate nucleic acid modifications) are particularly desirable and may be used in the same capacity as their corresponding siRNAs for all uses and methods disclosed herein.

It is to be understood that, in the context of the present invention, any of the siRNA molecules disclosed herein, or any long double-stranded RNA molecules (typically 25-500 nucleotides in length) which are processed by endogenous cellular complexes (such as DICER—see above) to form the siRNA molecules disclosed herein, or molecules which comprise the siRNA molecules disclosed herein, can be employed in the treatment of any disease or disorder. More specifically, the present invention provides a method of treating a patient suffering from a disease or disorder, such as nerodegenerative disorders or Central nervous system disorders, inter alia., comprising administering to the patient a pharmaceutical composition comprising one or more of the Annexin II siRNAs disclosed herein (or one or more long dsRNA which encodes one or more of said siRNAs, as described above) in a therapeutically effective amount so as to thereby treat the patient.

Additional disorders which can be treated by the molecules of the present invention, include Myocardial infarcation (MI) and apoptosis-related diseases described herein. An additional aspect of the present invention provides for methods of treating an apoptosis related disease. Methods for therapy of diseases or disorders associated with uncontrolled, pathological cell growth, e.g. cancer, psoriasis, autoimmune diseases, inter alia, and methods for therapy of diseases associated with ischemia and lack of proper blood flow, e.g. myocardial infarction (MI) and stroke, are provided.

Thus, in this aspect the invention provides a method of treating a patient suffering from MI, comprising administering to the patient a pharmaceutical composition comprising an Annexin II inhibitor in a therapeutically effective amount so as to thereby treat the patient. The inhibitor may comprise a small chemical compound, such as sodium nitroprusside (Liu et al., Eur. J. Biochem. 269, 4277-4286 (2002)), or the tyrosine kinase inhibitor Tyrphostin AG1024 which inhibits Annexin II secretion (Zhao et al., JBC 278, 6: 4205-4215 (2003)); a polynucleotide, such as a polynucleotide which comprises consecutive nucleotides having a sequence of sufficient length and homology to a sequence present within the sequence of the Annexin II gene set forth in SEQ ID NO:1 to permit hybridization of the inhibitor to the gene, optionally having one of the sequences set forth in FIG. 3 (SEQ ID NO:3 or SEQ ID NO:4), or a polynucleotide which is a sense polynucleotide comprising consecutive nucleotides having a sequence which is a sense sequence to the sequence set forth in FIG. 1 (SEQ ID NO:1), and which encodes a dominant negative peptide to said sequence, optionally having one of the sequences set forth in FIG. 4 (SEQ ID NO:5 or SEQ ID NO:6) or a polynucleotide which is an siRNA, optionally an siRNA comprising consecutive nucleotides having a sequence identical to any one of the sequences set forth in Tables 1-3 (SEQ ID NOs: 7-368) and in particular, siRNA No's 1-5 of Table 1, 100-107 of Table 2 and 174-181 of Table 3; a vector comprising any of these polynucleotides; a polypeptide, such as a dominant negative peptide, for example, the peptide encoded by SEQ ID NO:5 or SEQ ID NO:6, peptide #41 of PCT patent application publication No. WO 200404/1844 which was found to bind annexin II, S-nitrosogluthathione (GSNO; Liu et al., Eur. J. Biochem. 269, 4277-4286 (2002)) or an antibody which specifically binds to an epitope present within a polypeptide which comprises consecutive amino acids, the sequence of which is set forth in FIG. 2 (SEQ ID No:2)., optionally a polyclonal or a monoclonal antibody; or a ribozyme. The pharmaceutical composition may further contain a diluent or carrier.

An additional method of the present invention provides for a method for treating a patient suffering from MI, comprising administering to the patient a pharmaceutical composition comprising a therapeutically effective amount of an Annexin II inhibitor, such as any of the inhibitors described herein, so as to thereby treat the patient.

The apoptosis-related disease treatment aspect of the present invention also provides for the use of a therapeutically effective amount of an Annexin II inhibitor for the preparation of a medicament for promoting recovery in a patient suffering from a cancer or MI. The inhibitor may be one or more of the options detailed herein.

“Cancer” or “Tumor” refers to an uncontrolled growing mass of abnormal cells. These terms include both primary tumors, which may be benign or malignant, as well as secondary tumors, or metastases which have spread to other sites in the body. Examples of cancer-type diseases include, inter alia: carcinoma (e.g.: breast, colon and lung), leukemia such as B cell leukemia, lymphoma such as B-cell lymphoma, blastoma such as neuroblastoma and melanoma.

The term “Expression vector” refers to vectors that have the ability to incorporate and express heterologous DNA fragments in a foreign cell. Many prokaryotic and eukaryotic expression vectors are known and/or commercially available. Selection of appropriate expression vectors is within the knowledge of those having skill in the art.

By “Polypeptide” is meant a molecule composed of amino acids and the term includes peptides, polypeptides, proteins and peptidomimetics.

A peptidomimetic is a compound containing non-peptidic structural elements that is capable of mimicking the biological action(s) of a natural parent peptide. Some of the classical peptide characteristics such as enzymatically scissile peptidic bonds are normally not present in a peptidomimetic.

The term “amino acid” refers to a molecule which consists of any one of the 20 naturally occurring amino acids, amino acids which have been chemically modified (see below), or synthetic amino acids.

The term “dominant negative peptide” refers to a polypeptide encoded by a cDNA fragment that encodes for a part of a protein which can interact with the full protein and inhibit its activity or which can interact with other proteins and inhibit their activity in response to the full protein.

The term “antibody” refers to IgG, IgM, IgD, IgA, and IgE antibody, inter alia. The definition includes polyclonal antibodies or monoclonal antibodies. This term refers to whole antibodies or fragments of the antibodies comprising the antigen-binding domain of the anti-GPCRV product antibodies, e.g. antibodies without the Fc portion, single chain antibodies, fragments consisting of essentially only the variable, antigen-binding domain of the antibody, etc. The term “antibody” may also refer to antibodies against nucleic acid sequences obtained by cDNA vaccination.

The term also encompasses antibody fragments which retain the ability to selectively bind with their antigen or receptor and are exemplified as follows, inter alia:
(1) Fab, the fragment which contains a monovalent antigen-binding fragment of an antibody molecule which can be produced by digestion of whole antibody with the enzyme papain to yield a light chain and a portion of the heavy chain;
(2) (Fab′)₂, the fragment of the antibody that can be obtained by treating whole antibody with the enzyme pepsin without subsequent reduction; F(ab′₂) is a dimer of two Fab fragments held together by two disulfide bonds;
(3) Fv, defined as a genetically engineered fragment containing the variable region of the light chain and the variable region of the heavy chain expressed as two chains; and
(4) Single chain antibody (SCA), defined as a genetically engineered molecule containing the variable region of the light chain and the variable region of the heavy chain linked by a suitable polypeptide linker as a genetically fused single chain molecule.

By the term “epitope” as used in this invention is meant an antigenic determinant on an antigen to which the antibody binds. Epitopic determinants usually consist of chemically active surface groupings of molecules such as amino acids or sugar side chains and usually have specific three dimensional structural characteristics, as well as specific charge characteristics.

In one embodiment of the invention, any one of these pharmaceutical compositions is used for alleviation or reduction of the symptoms and signs associated with damaged neuronal tissues whether resulting from tissue trauma, or from chronic degenerative changes. This embodiment concerns a method or process for reducing damage to the central nervous system or promoting recovery in a patient who has suffered an injury to the central nervous system, comprising administering to the patient any one of the pharmaceutical compositions recited above, in a dosage and over a period of time sufficient to reduce the damage or promote recovery. This embodiment further provides a method or process for treating a patient who has suffered an injury to the central nervous system, optionally as a result of any of the conditions or injuries described herein, comprising administering to the patient a pharmaceutical composition comprising a therapeutically effective amount of a Annexin II inhibitor, as exemplified herein, in a dosage and over a period of time sufficient to inhibit Annexin II so as to thereby treat the patient.

It is known in the art, that in certain neurological diseases (for example, brain ischemia or stroke), the blood brain barrier (BBB) is relatively open compared to that of a normal subject, thus enabling penetration of molecules to the brain, even large molecules such as macromolecules, including antibodies, which would subsequently allow interaction of said molecules with Annexin II. Further information on delivery into the brain is provided in Example 8 below.

In one aspect of this invention, the injury to the central nervous system which said pharmaceutical composition is aimed at reducing, or from which said pharmaceutical composition is attempting to promote recovery, is an ischemic episode, which may be, but is not limited to, a global or focal cerebral episode; said injury may be a stroke event or a traumatic brain injury, as discussed herein. Further information on injuries or traumas of the CNS is provided below.

In another aspect of this invention, an additional pharmaceutically effective compound is administered in conjunction with the aforementioned pharmaceutical composition.

By “in conjunction with” is meant that the additional pharmaceutically effective composition is administered prior to, at the same time as, or subsequent to administration of the pharmaceutical composition comprising an Annexin II inhibitor.

In an additional embodiment of the present invention, any one of the above pharmaceutical compositions is used for causing regeneration of neurons in a subject in need thereof. This embodiment of the present invention concerns a method for causing regeneration of neurons in a patient in need thereof, comprising administering to the patient any one of the pharmaceutical compositions recited above, in a dosage and over a period of time sufficient to reduce the damage or promote recovery.

The pharmaceutical compositions of the present invention can have application in the treatment of any disease in which neuronal degeneration or damage is involved or implicated, such as, inter alia—the following conditions: hypertension, hypertensive cerebral vascular disease, a constriction or obstruction of a blood vessel-as occurs in the case of a thrombus or embolus, angioma, blood dyscrasias, any form of compromised cardiac function including cardiac arrest or failure, systemic hypotension; and diseases such as stroke, Parkinson's disease, epilepsy, depression, ALS, Alzheimer's disease, Huntington's disease and any other disease-induced dementia (such as HIV induced dementia for example). These conditions are also referred to herein as “neurodegenerative diseases”. Trauma to the central nervous system, such as rupture of aneurysm, cardiac arrest, cardiogenic shock, septic shock, spinal cord trauma, head trauma, traumatic brain injury (TBI), seizure, bleeding from a tumor, etc., are also referred to herein as “injury to the central nervous system” and may also be treated using the compounds and compositions of the present invention.

One embodiment of the claimed invention provides for using a therapeutically effective amount of a Annexin II inhibitor in a process for the preparation of a medicament for the treatment of a patient who has suffered an injury to the central nervous system such as, inter alia, an ischemic episode, a stroke or a traumatic brain injury. The inhibitor may be a small chemical compound, such as sodium nitroprusside (Liu et al., Eur. J. Biochem. 269, 4277-4286 (2002)), or the tyrosine kinase inhibitor Tyrphostin AG1024 which inhibits AnnexinII secretion (Zhao et al., JBC 278, 6: 4205-4215 (2003)); a polynucleotide, such as an antisense polynucleotide comprising consecutive nucleotides having a sequence which is an antisense sequence to the sequence set forth in FIG. 1 (SEQ ID NO:1), optionally having one of the sequences set forth in FIG. 3 (SEQ ID NO:3 or SEQ ID NO:4), or a polynucleotide which is a sense polynucleotide comprising consecutive nucleotides having a sequence which is a sense sequence to the sequence set forth in FIG. 1 (SEQ ID NO:1), and which encodes a dominant negative peptide to said sequence, optionally having one of the sequences set forth in FIG. 4 (SEQ ID NO:5 or SEQ ID NO:6) or a polynucleotide that functions as silencing RNA (siRNA), optionally having one of the sequence set forth in tables 1-3, particularly in Table 1; a vector comprising any of these polynucleotides; a polypeptide, such as a dominant negative peptide, for example, the peptide encoded by SEQ ID NO:5 or SEQ ID NO:6, peptide #41 of PCT patent application publication No. WO 200404/1844 which was found to bind annexin II, S-nitrosogluthathione (GSNO; Liu et al., Eur. J. Biochem. 269, 4277-4286 (2002)) or an antibody, optionally a polyclonal or a monoclonal antibody, such as the antibody detailed above. The pharmaceutical composition may further contain a diluent or carrier.

The treatment regimen according to the invention is carried out, in terms of administration mode, timing of the administration, and dosage, so that the functional recovery of the patient from the adverse consequences of the ischemic events or central nervous system injury is improved; i.e., at least one of the patient's motor skills (e.g., posture, balance, grasp, or gait), cognitive skills, speech, and/or sensory perception (including visual ability, taste, olfaction, and proprioception) improve as a result of inhibitor administration according to the invention. Thus the inhibitor promotes or enhances recovery of the patient by improving at least one of these skills.

Administration of a pharmaceutical composition comprising an Annexin II inhibitor according to the invention can be carried out by any known route of administration, including intravenously, intra-arterially, subcutaneously, or intracerebrally. Using specialized formulations, it may also be possible to administer these orally or via inhalation. Suitable doses and treatment regimens for administering compositions to an individual in need thereof are discussed in detail below.

The invention can be used to treat the adverse consequences of central nervous system injuries that result from any of a variety of conditions. Thrombus, embolus, and systemic hypotension are among the most common causes of cerebral ischemic episodes. Other injuries may be caused by hypertension, hypertensive cerebral vascular disease, rupture of an aneurysm, an angioma, blood dyscrasias, cardiac failure, cardiac arrest, cardiogenic shock, septic shock, head trauma, spinal cord trauma, seizure, bleeding from tumor, or other blood loss.

Where the ischemia is associated with stroke, it can be either global or focal ischemia, as defined below. It is believed that the administration of a pharmaceutical composition according to the invention is effective, even though administration occurs a significant amount of time following the injury.

By “ischemic episode” is meant any circumstance that results in a deficient supply of blood to a tissue. Cerebral ischemic episodes result from a deficiency in the blood supply to the brain. The spinal cord, which is also part of the central nervous system, is equally susceptible to ischemia resulting from diminished blood flow. An ischemic episode may be caused by hypertension, hypertensive cerebral vascular disease, rupture of aneurysm, a constriction or obstruction of a blood vessel-as occurs in the case of a thrombus or embolus, angioma, blood dyscrasias, any form of compromised cardiac function including cardiac arrest or failure, systemic hypotension, cardiac arrest, cardiogenic shock, septic shock, spinal cord trauma, head trauma, seizure, bleeding from a tumor, or other blood loss. It is expected that the invention will also be useful for treating injuries to the central nervous system that are caused by mechanical forces, such as a blow to the head or spine. Trauma can involve a tissue insult such as an abrasion, incision, contusion, puncture, compression, etc., such as can arise from traumatic contact of a foreign object with any locus of or appurtenant to the head, neck, or vertebral column. Other forms of traumatic injury can arise from constriction or compression of CNS tissue by an inappropriate accumulation of fluid (for example, a blockade or dysfunction of normal cerebrospinal fluid or vitreous humor fluid production, turnover, or volume regulation, or a subdural or intracamial hematoma or edema). Similarly, traumatic constriction or compression can arise from the presence of a mass of abnormal tissue, such as a metastatic or primary tumor.

By “focal ischemia” as used herein in reference to the central nervous system, is meant the condition that results from the blockage of a single artery that supply blood to the brain or spinal cord, resulting in the death of all cellular elements (pan-necrosis) in the territory supplied by that artery.

By “global ischemia” as used herein in reference to the central nervous system, is meant the condition that results from general diminution of blood flow to the entire brain, forebrain, or spinal cord, which causes the death of neurons in selectively vulnerable regions throughout these tissues. The pathology in each of these cases is quite different, as are the clinical correlates. Models of focal ischemia apply to patients with focal cerebral infarction, while models of global ischemia are analogous to cardiac arrest, and other causes of systemic hypotension.

The term “neurotoxic stress” as used herein is intended to comprehend any stress that is toxic to normal neural cells (and may cause their death or apoptosis). Such stress may be oxidative stress (hypoxia or hyperoxia) or ischemia or trauma, and/or it may involve subjecting the cells to a substance that is toxic to the cells in vivo, such as glutamate or dopamine or the Aβ protein, or any substance or treatment that causes oxidative stress. The neurotoxic substance may be endogenous or exogenous and the term neurotoxic is also intended to comprehend exposure to various known neurotoxins including organophosphorous poisoning, or any other insult of this type. In addition, neurotoxic stress may be caused by a neurodegenerative disease.

In an additional embodiment, the present invention provides for a method or process for causing regeneration of neurons in a subject in need thereof, comprising administering to the subject a pharmaceutical composition which comprises a Annexin II inhibitor as an active ingredient in a therapeutically effective amount, further comprising a diluent or carrier and optionally being any of the pharmaceutical compostions as described herein.

An additional embodiment of the present invention, referred to herein as the “screening” embodiment, concerns methods and processes for obtaining a species and/or chemical compound that modulates the biological activity of Annexin II, neurotoxic stress and/or apoptosis. One aspect of this embodiment provides a process for obtaining a species and/or chemical compound that modulates the biological activity of Annexin II, neurotoxic stress and/or apoptosis which comprises contacting a cell expressing Annexin II with a species and/or compound and determining the ability of the species and/or compound to modulate the biological activity of Annexin II, neurotoxic stress and/or apoptosis of the cell as compared to a control. The cell being examined may be modified to express Annexin II, and—without being bound by theory—apoptosis may be induced by the presence of Annexin II, or by neurotoxic stress, optionally caused by hydrogen peroxide, glutamate, dopamine, the Aβ protein or any known neurotoxin or neurotoxic treatment such as ischemia or hypoxia, or by a neurodegenerative disease such as stroke. In addition, this process may be used in order to prepare a pharmaceutical composition. The process then comprises admixing a species or compound obtained by the process recited above or a chemical analog or homolog thereof with a pharmaceutically acceptable carrier.

By cells being “modified to express” as used herein is meant that cells are modified by transfection, transduction, infection or any other known molecular biology method which will cause the cells to express the desired gene. Materials and protocols for carrying out such methods are evident to the skilled artisan.

An additional aspect of the screening embodiment provides a process of screening a plurality of species or compounds to obtain a species and/or compound that modulates the biological activity of Annexin II, neurotoxic stress and/or apoptosis, which comprises:
(a) contacting cells expressing Annexin II with a plurality of species and/or chemical compounds;
(b) determining whether the biological activity of Annexin II, neurotoxic stress and/or apoptosis is modulated in the presence of the species and/or compounds, as compared to a control; and if so
(c) separately determining whether the modulation of the biological activity of Annexin II, neurotoxic stress and/or apoptosis is effected by each species and/or compound included in the plurality of species and/or compounds, so as to thereby identify the species and/or compound which modulates the biological activity of Annexin II, neurotoxic stress and/or apoptosis.

The cells in the contacting step may be modified to express the Annexin II polypeptide, and—without being bound by theory—apoptosis may be induced spontaneously by Annexin II overexpression, or as a result of subjection of the cells to neurotoxic stress, optionally caused by hydrogen peroxide, glutamate, dopamine, the AP protein or any known neurotoxin or neurotoxic treatment such as ischemia or hypoxia, or by a neurodegenerative disease such as stroke. In addition, this process may be used in order to prepare a pharmaceutical composition. The process then comprises admixing a species or compound identified by the process recited above or a chemical analog or homolog thereof with a pharmaceutically acceptable carrier.

The process may additionally comprise modification of a species or compound found to modulate apoptosis by the above process to produce a compound with improved activity and admixing such compound with a pharmaceutically acceptable carrier. This additional act may be performed with a compound discovered by any of the processes which are disclosed in the screening embodiment of the present invention, so as to thereby obtain a pharmaceutical composition comprising a compound with improved activity.

Additionally, the screening embodiment of the present invention provides a non cell-based process for obtaining a species or compound which modulates the biological activity of Annexin II, neurotoxic stress and/or apoptosis (through Annexin II) comprising:
(a) measuring the binding of Annexin II or the Annexin II gene to an interactor;
(b) contacting Annexin II or the Annexin II gene with said species or compound; and
(c) determining whether the binding of Annexin II or the Annexin II gene to said interactor is affected by said species or compound.

The in-vitro system may be subjected to apoptotic conditions, which can be induced-without being bound by theory-by causing neurotoxic stress, as a result of treatment with, inter alia, hydrogen peroxide, glutamate, dopamine, the Aβ protein or any known neurotoxin. In addition, this process may be used in order to prepare a pharmaceutical composition. The process then comprises admixing a species or compound identified by the process recited above or a chemical analog or homolog thereof with a pharmaceutically acceptable carrier.

Another aspect of the screening embodiment provided by the present invention is a kit for obtaining a species or compound which modulates the biological activity of Annexin II or the Annexin II gene, neurotoxic stress and/or apoptosis in a cell comprising:
(a) Annexin II or the Annexin II gene; and
(b) an interactor with which Annexin II or the Annexin II gene interacts;
(c) means for measuring the interaction of Annexin II or the Annexin II gene with the interactor; and
(d) means of determining whether the binding of Annexin II or the Annexin II gene to the interactor is affected by said species or compound.

Means of measuring interactions between molecules and determining the strength, affinity, avidity and other parameters of the interaction are well known in the art (see, for example, Lubert Stryer, Biochemistry, W H Freeman & Co.; 5th edition (April 2002); and “Comprehensive Medicinal Chemistry”, by various authors and editors, published by Pergamon Press).

An additional embodiment of the present invention concerns a method or process for diagnosing cells which have been subjected to neurotoxic stress and/or stroke and/or cancer, comprising assaying for RNA corresponding to a sequence comprised in SEQ ID NO:1 or a fragment or homolog thereof, or for the expression product of a gene in which one of said sequences is a part, the finding of up-regulation of said RNA or expression product as compared to a normal control indicating the likelihood that such cells have been subjected to neurotoxic stress and/or stroke, and further the finding of down-regulation of said RNA or expression product as compared to a normal control indicating the likelihood that such cells have been subjected to a cancer or become cancerous.

The present invention further provides a method or process for diagnosing a neurodegenerative disease in a subject comprising detecting modulation of the expression level of Annexin II (for example: by detecting Annexin II in an immunoassay) or the Annexin II gene (for example: by detecting an mRNA encoding Annexin II) in the subject, as compared to a control. In one embodiment, the subject being diagnosed is suspected to have undergone a stroke.

Another embodiment of the present invention concerns a method or process for diagnosing a neurodegenerative disease in a subject comprising detecting modulation of the expression level of the Annexin polypeptide in the subject as compared to a control, whereas said modulation of expression is indicative of the likelihood of neurodegenerative disease in the subject; indeed, the diagnostic methods of the present invention may be practiced on a subject suspected to have undergone a stroke.

The expression level of the polypeptide can be assessed by assaying for mRNA encoding the Annexin polypeptide (such as that described in FIG. 1 or, or a fragment or homolog thereof), or by method of an immunoassay using antibodies which detect the polypeptide. Both detection of mRNA and immunoassays can be performed by methods well known in the art. Measurement of level of the Annexin II polypeptide is determined by a method selected from the group consisting of immunohistochemistry (Microscopy, Immunohistochemistry and Antigen Retrieval Methods: For Light and Electron Microscopy, M. A. Hayat (Author), Kluwer Academic Publishers, 2002; Brown C.: “Antigen retrieval methods for immunohistochemistry”, Toxicol Pathol 1998; 26(6): 830-1), western blotting (Laemmeli UK: “Cleavage of structural proteins during the assembley of the head of a bacteriophage T4”, Nature 1970; 227: 680-685; Egger & Bienz, “Protein (western) blotting”, Mol Biotechnol 1994; 1(3): 289-305), ELISA (Onorato et al., “Immunohistochemical and ELISA assays for biomarkers of oxidative stress in aging and disease”, Ann NY Acad Sci 1998 20; 854: 277-90), antibody microarray hybridization (Huang, “detection of multiple proteins in an antibody-based protein microarray system, Immunol Methods 2001 1; 255 (1-2): 1-13) and targeted molecular imaging (Thomas, Targeted Molecular Imaging in Oncology, Kim et al (Eds)., Springer Verlag, 2001).

Measurement of level of Annexin II polynucleotide is determined by a method selected from: RT-PCR analysis, in-situ hybridization (“Introduction to Fluorescence In Situ Hybridization: Principles and Clinical Applications”, Andreeff & Pinkel (Editors), John Wiley & Sons Inc., 1999), polynucleotide microarray and Northern blotting (Trayhurn, “Northern blotting”, Proc Nutr Soc 1996; 55(1B): 583-9; Shifman & Stein, “A reliable and sensitive method for non-radioactive Northern blot analysis of nerve growth factor mRNA from brain tissues”, Journal of Neuroscience Methods 1995; 59: 205-208). This diagnostic method may be useful, inter alia, for diagnosing patients suspected to have undergone a stroke.

By “abnormal” in the context of protein expression, is meant a difference of at least 10% in the expression levels of the polypeptide as compared to a control.

Additionally, the invention provides a method or process of treating a tumor or an auto-immune disease in a subject which comprises administering to the subject a therapeutically effective amount of a pharmaceutical composition which modulates the biological activity of Annexin II.

Further, the invention provides a method or process of treating neurodegenerative disease in a subject which comprises administering to the subject a therapeutically effective amount of a pharmaceutical composition which inhibits the biological activity of Annexin II.

The invention further provides for the use of an Annexin II modulator in the preparation of a medicament; said medicament may be used for the treatment of a neurodegenerative disease.

Another embodiment of the present invention provides for a substantially purified polynucleotide comprising consecutive nucleotides having any one of the sequences described in FIG. 3 or 4, i.e., SEQ ID NO:3, SEQ ID NO:4, SEQ ID NO:5 SEQ ID NO:6, or a sequence at least 70% homologous to any one of said sequences, or any of the siRNA sequences disclosed in Tables 1-3, particularly in table 1, and a vector which comprises any one of said polynucleotides. Said vector may be of a specific type aimed at gene therapy or targeting.

Another aspect of the present invention deals with the use of Annexin II for its capacity to enhance apoptosis. In this aspect, the invention provides for a method or process of treating a tumor or auto-immune disease in a subject by administering to the subject a therapeutically effective amount of a chemical compound, wherein the chemical compound comprises Annexin II, or the Annexin II cDNA, or a therapeutically effective amount of a chemical compound which stimulates the Annexin II cDNA or polypeptide, all separately or in combination. In this aspect, the invention further provides for the use of Annexin II or a vector comprising the Annexin II cDNA for the preparation of a medicament for promoting or enhancing recovery in a patient suffering from a tumor or auto-immune disease.

It will be noted that all the polynucleotides to be used in the present invention may undergo modifications so as to possess improved therapeutic properties. Modifications or analogs of nucleotides can be introduced to improve the therapeutic properties of polynucleotides. Improved properties include increased nuclease resistance and/or increased ability to permeate cell membranes. Nuclease resistance, where needed, is provided by any method known in the art that does not interfere with biological activity of the AS polynucleotide, siRNA, cDNA and/or ribozymes as needed for the method of use and delivery (Iyer et al., 1990; Eckstein, 1985; Spitzer and Eckstein, 1988; Woolf et al., 1990; Shaw et al., 1991). Modifications that can be made to oligonucleotides in order to enhance nuclease resistance include modifying the phophorous or oxygen heteroatom in the phosphate backbone. These include preparing methyl phosphonates, phosphorothioates, phosphorodithioates and morpholino oligomers. In one embodiment it is provided by having phosphorothioate bonds linking between the four to six 3′-terminus nucleotide bases. Alternatively, phosphorothioate bonds link all the nucleotide bases. Other modifications known in the art may be used where the biological activity is retained, but the stability to nucleases is substantially increased.

All analogues of, or modifications to, a polynucleotide may be employed with the present invention, provided that said analogue or modification does not substantially affect the function of the polynucleotide. The nucleotides can be selected from naturally occurring or synthetic modified bases. Naturally occurring bases include adenine, guanine, cytosine, thymine and uracil. Modified bases of nucleotides include inosine, xanthine, hypoxanthine, 2-aminoadenine, 6-methyl, 2-propyl and other alkyl adenines, 5-halo uracil, 5-halo cytosine, 6-aza cytosine and 6-aza thymine, psuedo uracil, 4-thiuracil, 8-halo adenine, 8-aminoadenine, 8-thiol adenine, 8-thiolalkyl adenines, 8-hydroxyl adenine and other 8-substituted adenines, 8-halo guanines, 8-amino guanine, 8-thiol guanine, 8-thioalkyl guanines, 8-hydroxyl guanine and other substituted guanines, other aza and deaza adenines, other aza and deaza guanines, 5-trifluoromethyl uracil and 5-trifluoro cytosine.

In addition, analogues of polynucleotides can be prepared wherein the structure of the nucleotide is fundamentally altered and that are better suited as therapeutic or experimental reagents. An example of a nucleotide analogue is a peptide nucleic acid (PNA) wherein the deoxyribose (or ribose) phosphate backbone in DNA (or RNA is replaced with a polyamide backbone which is similar to that found in peptides. PNA analogues have been shown to be resistant to degradation by enzymes and to have extended lives in vivo and in vitro. Further, PNAs have been shown to bind stronger to a complementary DNA sequence than a DNA molecule. This observation is attributed to the lack of charge repulsion between the PNA strand and the DNA strand. Other modifications that can be made to oligonucleotides include polymer backbones, cyclic backbones, or acyclic backbones.

The polypeptides employed in the present invention may also be modified, optionally chemically modified, in order to improve their therapeutic activity. “Chemically modified”—when referring to the polypeptides, means a polypeptide where at least one of its amino acid residues is modified either by natural processes, such as processing or other post-translational modifications, or by chemical modification techniques which are well known in the art. Among the numerous known modifications typical, but not exclusive examples include: acetylation, acylation, amidation, ADP-ribosylation, glycosylation, GPI anchor formation, covalent attachment of a lipid or lipid derivative, methylation, myristlyation, pegylation, prenylation, phosphorylation, ubiqutination, or any similar process.

Additional possible polypeptide modifications (such as those resulting from nucleic acid sequence alteration) include the following:

“Conservative substitution”—refers to the substitution of an amino acid in one class by an amino acid of the same class, where a class is defined by common physicochemical amino acid side chain properties and high substitution frequencies in homologous polypeptides found in nature, as determined, for example, by a standard Dayhoff frequency exchange matrix or BLOSUM matrix. Six general classes of amino acid side chains have been categorized and include: Class I (Cys); Class II (Ser, Thr, Pro, Ala, Gly); Class III (Asn, Asp, Gln, Glu); Class IV (His, Arg, Lys); Class V (Ile, Leu, Val, Met); and Class VI (Phe, Tyr, Trp). For example, substitution of an Asp for another class III residue such as Asn, Gln, or Glu, is a conservative substitution.

“Non-conservative substitution”—refers to the substitution of an amino acid in one class with an amino acid from another class; for example, substitution of an Ala, a class II residue, with a class III residue such as Asp, Asn, Glu, or Gln.

“Deletion”—is a change in either nucleotide or amino acid sequence in which one or more nucleotides or amino acid residues, respectively, are absent.

“Insertion” or “addition”—is that change in a nucleotide or amino acid sequence which has resulted in the addition of one or more nucleotides or amino acid residues, respectively, as compared to the naturally occurring sequence.

“Substitution”—replacement of one or more nucleotides or amino acids by different nucleotides or amino acids, respectively. As regards amino acid sequences the substitution may be conservative or non-conservative.

In an additional embodiment of the present invention, the Annexin II polypeptide or polynucleotide may be used to diagnose or detect macular degeneration in a subject. A detection method would typically comprise assaying for Annexin II mRNA or Annexin II polypeptide in a sample derived from a subject.

“Detection”—refers to a method of detection of a disease. This term may refer to detection of a predisposition to a disease, or to the detection of the severity of the disease.

By “homolog/homology”, as utilized in the present invention, is meant at least about 70%, preferably at least about 75% homology, advantageously at least about 80% homology, more advantageously at least about 90% homology, even more advantageously at least about 95%, e.g., at least about 97%, about 98%, about 99% or even about 100% homology. The invention also comprehends that these polynucleotides and polypeptides can be used in the same fashion as the herein or aforementioned polynucleotides and polypeptides.

Alternatively or additionally, “homology”, with respect to sequences, can refer to the number of positions with identical nucleotides or amino acid residues, divided by the number of nucleotides or amino acid residues in the shorter of the two sequences, wherein alignment of the two sequences can be determined in accordance with the Wilbur and Lipman algorithm ((1983) Proc. Natl. Acad. Sci. USA 80:726); for instance, using a window size of 20 nucleotides, a word length of 4 nucleotides, and a gap penalty of 4, computer-assisted analysis and interpretation of the sequence data, including alignment, can be conveniently performed using commercially available programs (e.g., Intelligenetics™ Suite, Intelligenetics Inc., CA). When RNA sequences are said to be similar, or to have a degree of sequence identity or homology with DNA sequences, thymidine (T) in the DNA sequence is considered equal to uracil (U) in the RNA sequence. RNA sequences within the scope of the invention can be derived from DNA sequences or their complements, by substituting thymidine (T) in the DNA sequence with uracil (U).

Additionally or alternatively, amino acid sequence similarity or homology can be determined, for instance, using the BlastP program (Altschul et al., Nucl. Acids Res. 25:3389-3402) and available at NCBI. The following references provide algorithms for comparing the relative identity or homology of amino acid residues of two polypeptides, and additionally, or alternatively, with respect to the foregoing, the teachings in these references can be used for determining percent homology: Smith et al., (1981) Adv. Appl. Math. 2:482-489; Smith et al., (1983) Nucl. Acids Res. 11:2205-2220; Devereux et al., (1984) Nucl. Acids Res. 12:387-395; Feng et al., (1987) J.

Molec. Evol. 25:351-360; Higgins et al., (1989) CABIOS 5:151-153; and Thompson et al., (1994) Nucl. Acids Res. 22:4673-4680.

“Having at least X % homolgy”—with respect to two amino acid or nucleotide sequences, refers to the percentage of residues that are identical in the two sequences when the sequences are optimally aligned. Thus, 90% amino acid sequence identity means that 90% of the amino acids in two or more optimally aligned polypeptide sequences are identical.

By the term “modulates” in the context of apoptosis modulation is meant either increases (promotes, enhances) or decreases (prevents, inhibits).

The invention has been described in an illustrative manner, and it is to be understood that the terminology which has been used is intended to be in the nature of words of description rather than of limitation.

Obviously, many modifications and variations of the present invention are possible in light of the above teachings. It is, therefore, to be understood that within the scope of the appended claims, the invention can be practiced otherwise than as specifically described.

Throughout this application, various publications, including United States patents, are referenced by author and year and patents by number. The disclosures of these publications and patents and patent applications in their entireties are hereby incorporated by reference into this application in order to more fully describe the state of the art to which this invention pertains.

### BRIEF DESCRIPTION OF THE FIGURES

FIG. 1. This figure sets forth the nucleotide sequence of the human Annexin II gene cDNA—SEQ ID NO:1.

FIG. 2. This figure sets forth the amino acid sequence of the human Annexin II corresponding polypeptide—SEQ ID NO:2.

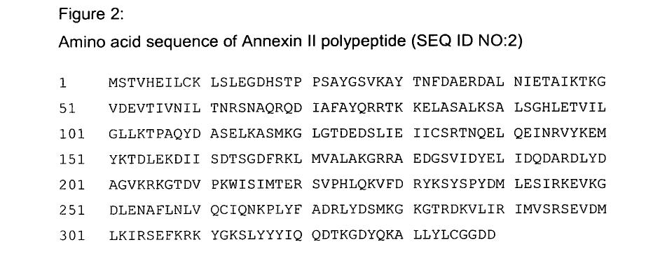

FIG. 3. This figure sets forth the nucleotide sequence of two Annexin II antisense fragments (SEQ ID NO:3 and SEQ ID NO:4);

FIG. 4. This figure sets forth the nucleotide sequence of two Annexin II sense fragments (SEQ ID NO:5 and SEQ ID NO:6);

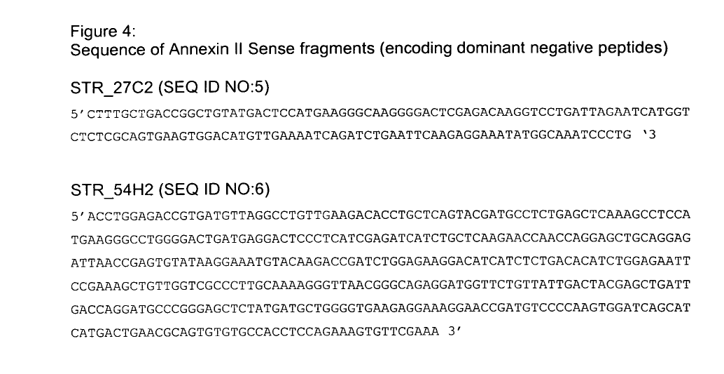

FIG. 5. This figure is a graph illustrating the results of a loss of function validation experiment.

### EXAMPLES

Without further elaboration, it is believed that one skilled in the art can, using the preceding description, utilize the present invention to its fullest extent. The following preferred specific embodiments are, therefore, to be construed as merely illustrative, and not limitative of the claimed invention in any way.

Standard molecular biology protocols known in the art not specifically described herein are generally followed essentially as in Sambrook et al., Molecular cloning: A laboratory manual, Cold Springs Harbor Laboratory, New-York (1989, 1992), and in Ausubel et al., Current Protocols in Molecular Biology, John Wiley and Sons, Baltimore, Md. (1988).

Standard organic synthesis protocols known in the art not specifically described herein are generally followed essentially as in Organic syntheses: Vol. 1-79, editors vary, J. Wiley, New York, (1941-2003); Gewert et al., Organic synthesis workbook, Wiley-VCH, Weinheim (2000); Smith & March, Advanced Organic Chemistry, Wiley-Interscience; 5th edition (2001).

Standard medicinal chemistry methods known in the art not specifically described herein are generally followed essentially as in the series “Comprehensive Medicinal Chemistry”, by various authors and editors, published by Pergamon Press.

### Example 1

### Identification of Genes Involved in the Stroke Event—Annexin II

As a first step to the novel drug discovery, key genes involved in the stroke event were identified, as provided by the following methods:

Summary of cDNA Micro-Array Construction

The polynucleotide encoding Annexin II was found by microarray-based differential gene expression, evaluated by both in vivo and in vitro models.

The cDNA microarray was constructed by combining cDNA libraries (Table A), including a subtraction library, enriched for stroke specific genes. As a result, the “Stroke Chip” consists of a microarray imprinted with about 10,000 low-redundant stroke-specific cDNA clones. The libraries printed on the chip were as described in Table A.

TABLE A
Design of the Stroke Chip: Library types and cDNA sources.

| | Material | | Time points | | | |
|---|---|---|---|---|---|---|
| Type of Library | In vivo | In vitro | 3 h | 6 h | 16 h | 24 h |
| Subtraction library | [MCAO] − [Sham] | | +L3 | +L4 | | |
| (five independent | [MCAO + FK506] − [MCAO] | | +L5 | +L6 | | |
| libraries) | | Primary neurons: | +L1 | +L1 | +L1 | +L1 |
| | | [Hypoxia + FK506] − [Normoxia + FK506] | | | | |
| SDGI library | MCAO | | +L7 | +L8 | | |
| (pool of 6 conditions) | MCAO + FK506 | | +L9 | | | |
| | Sham + FK506 | | +L10 | | | |
| | | Primary neurons: | +L2 | +L2 | +L2 | +L2 |
| | | [Hypoxia] | | | | |
| | | Primary neurons: | +L11 | | | |
| | | [Hypoxia + FK506] | | | | |

Each library is indicated by L and numbered. Middle cerebral artery occlusion (MCAO) was performed in SD rats and primary neurons are rat cortical primary neurons. Normoxia indicates normal oxygen concentration.

FK506 (tacrolimus) is a known immunosuppressive agent produced by Streptomyces tsukubaesis. FK506 possesses neuroprotective activity by delaying or preventing hypoxia-induced death of neuronal cells. In addition, it can cause re-growth of damaged nerve cells. The specific molecular mechanism underlying the neuroprotective activity of FK506 is largely unknown although there are indications for suppression of activities of calcineurin and nitric oxide synthase as well as prevention of stroke induced generation of ceramide and Fas signaling. In the present invention, FK 506 serves for pinpointing genes that are not only regulated by ischemic-induced danage but are also regulated by the addition of FK-506.

The libraries imprinted on the Stroke Chip were constructed as follows:

a) Subtractive libraries: An ischemia (stroke) model was created in SD and SHR rats by permanent middle cerebral artery occlusion (MCAO). Control rats of the same strain were subjected to a sham operation (Sham). Half of the rats of each group were given FK506 treatment at 0 hour. Subtraction libraries comprised genes expressed in the MCAO rats but not in the sham operated rats (MCAO—Sham), and those genes expressed in the MCAO rats treated with FK506 (taken at 3 hours and 6 hours after FK506 treatment) but not in the MCAO treated rats ([MCAO+FK506]−[MCAO]). Another library included in the Stroke Chip was derived from in vitro treatment of primary neurons from the cerebellum of 7-day rat pups. The cells were subjected to hypoxia (0.5% O₂) for 16 hours. The cells under hypoxia and control cells under normal oxygen concentration (normoxia) were treated with FK506 (100 ng/ml) at 0 hour and the cDNA extracted after 16 hours. A subtraction library was made from the cDNA fragments expressed in the FK506 treated cells under hypoxia but not in the FK506 treated cells under normoxia ([Hypoxia+FK506]−[Normoxia+FK506]).

b) Libraries generated by sequence-dependent gene identification (SDGI). This technique is essentially as described in PCT application no. PCT/US01/09392. SDGI libraries were prepared from brain tissues of the rats subjected to MCAO, MCAO rats three and six hours after treatment with FK506, and sham operated rats three and six hours after treatment with FK506. SDGI libraries were also prepared from primary neurons that were subjected to hypoxia for 16 hours in the in vitro experiments and from primary neurons, pretreated with FK506 and subjected to hypoxia for 16 hours.

Thus, the cDNA libraries used in the preparation of the stroke chip were prepared as described above, and so were enriched for cDNAs that are differentially expressed in stroke by either subtractive hybridization (SSH) and/or sequence-dependent gene identification (SDGI).

The stroke chip was used for differential hybridization experiments as described below.

Hybridizations to the Stroke Chip

Cells either in vivo or in vitro were subjected to a developmental, physiological, pharmacological or other cued event that would cause genes to be activated or repressed in response thereto (this gene expression array technology was disclosed, for example in U.S. Pat. No. 5,807,522), and probes were produced; production of probes and their use in interrogating a microarray chip is described for example in U.S. Pat. No. 6,291,170.

Hybridizations were performed according to the following:

Probes used for hybridizations on the Stroke chip were prepared using Four paired groups of animals treated by the following treatments:
1. Animals that in addition to MCAO received FK-506 were sacrificed at 1.5, 3 and 6 hours. The cortex of these animals was removed and used for probe generation
2. Animals that in addition to MCAO received FK-506 were sacrificed at 1.5, 3 and 6 hours. The whole ipsilateral hemisphere (the operated side) of these animals was removed and used for probe generation
3. Animals that in addition to MCAO received vehicle were sacrificed at 1.5, 3, 6, 12, 24 and 48 hours. The ipsilateral cortex of these animals was removed and used for probe generation
4. Animals that in addition to MCAO received FK-506 were sacrificed at 1.5, 3, 6, 12, 24 and 48 hours. The whole ipsilateral hemisphere of these animals was removed and used for probe generation.
The probes were labeled and hybridized to the stroke chip.
In addition to these probes, a common control probe labeled with Cy3 was added to each hybridization. The common control probes were mixtures of poly-A RNA extracted from the whole brain of SD rats.

Preparation of Tissues for In Situ Analysis
Coronal sections were prepared from paraffin blocks of sham operated rat brains and brains subjected to MCAO.

The model was characterized using hybridization of control genes known to be affected in stroke such as c-fos and p21 and staining of sections with microtubule associated protein 2 (stains neuronal cell body and dendrites indicating the integrity of neuronal cell cytoskeleton) GFAP (glial filament associated protein); this staining is specific for astrocytes and not myelinating oligodendrocytes and indicates the integrity of glial cell cytoskeleton. Results of these hybridizations were consistent with previously reported results. Thus, suitability of obtained paraffin blocks for in situ hybridization study and suitability of the model for this study were demonstrated.

Summary of the Results

As a result of screening of the stroke chip, the expression of Annexin II was found to be induced upon 6 h of MCAO. The induction reached a maximum at 48 h (cortex-3.5 fold, whole hemispher-5.5 fold).

Results of the in situ hybridization study suggest constitutive expression of the Annexin II gene in ependimal and meningothelial cells. MCAO results in the accumulation of leucocytes and macrophages expressing Annexin II. No activation of the Annexin II expression was found in neurons.

### Example 2

### General Methods

General Methods in Molecular Biology

Standard molecular biology techniques known in the art and not specifically described were generally followed as in Sambrook et al., Molecular Cloning: A Laboratory Manual, Cold Springs Harbor Laboratory, New York (1989, 1992), and in Ausubel et al., Current Protocols in Molecular Biology, John Wiley and Sons, Baltimore, Md. (1989).

Polymerase chain reaction (PCR) was carried out generally as in PCR Protocols: A Guide To Methods And Applications, Academic Press, San Diego, Calif. (1990). Reactions and manipulations involving other nucleic acid techniques, unless stated otherwise, were performed as generally described in Sambrook et al., 1989, Molecular Cloning: A Laboratory Manual, Cold Spring Harbor Laboratory Press, and methodology as set forth in U.S. Pat. Nos. 4,666,828; 4,683,202; 4,801,531; 5,192,659 and 5,272,057 and incorporated herein by reference.

Protein Purification is performed as described below in Example 6.

Vectors are constructed containing the cDNA of the present invention by those skilled in the art and can contain all expression elements necessary to achieve the desired transcription of the sequences, should transcription be required (see below in specific methods for a more detailed description). Other beneficial characteristics can also be contained within the vectors such as mechanisms for recovery of the nucleic acids in a different form. Phagemids are a specific example of such beneficial vectors because they can be used either as plasmids or as bacteriophage vectors. Examples of other vectors include viruses such as bacteriophages, baculoviruses and retroviruses, DNA viruses, cosmids, plasmids, liposomes and other recombination vectors. The vectors can also contain elements for use in either procaryotic or eucaryotic host systems. One of ordinary skill in the art knows which host systems are compatible with a particular vector.

The vectors are introduced into cells or tissues by any one of a variety of known methods within the art (calcium phosphate transfection; electroporation; lipofection; protoplast fusion; polybrene transfection). The host cell can be any eucaryotic and procaryotic cells, which can be transformed with the vector and which supports the production of the polypeptide. Methods for transformation can be found generally described in Sambrook et al., Molecular Cloning: A Laboratory Manual, Cold Springs Harbor Laboratory, New York (1992), in Ausubel et al., Current Protocols in Molecular Biology, John Wiley and Sons, Baltimore, Md. (1989), Chang et al., Somatic Gene Therapy, CRC Press, Ann Arbor, Mich. (1995), Vega et al., Gene Targeting, CRC Press, Ann Arbor, Mich. (1995) and Gilboa, et al. (1986) and include, for example, stable or transient transfection, lipofection, electroporation and infection with recombinant viral vectors. In addition, see U.S. Pat. No. 4,866,042 for vectors involving the central nervous system and also U.S. Pat. Nos. 5,464,764 and 5,487,992 for positive-negative selection methods.

General Methods in Immunology

Standard methods in immunology known in the art and not specifically described were generally followed as in Stites et al.(eds), Basic and Clinical Immunology (8th Edition), Appleton & Lange, Norwalk, Conn. (1994) and Mishell and Shiigi (eds), Selected Methods in Cellular Immunology, W.H. Freeman and Co., New York (1980).

Immunoassays

In general, ELISAs are the preferred immunoassays employed to assess a specimen. ELISA assays are well known to those skilled in the art. Both polyclonal and monoclonal antibodies can be used in the assays. Where appropriate other immunoassays, such as radioimmunoassays (RIA) can be used as are known to those in the art. Available immunoassays are extensively described in the patent and scientific literature. See, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521 as well as Sambrook et al, Molecular Cloning: A Laboratory Manual, Cold Springs Harbor, N.Y., 1989.

### Example 3

### Experimental Validation Results

siRNA was used for the validation; utilizing siRNA, one can inhibit or reduce the level of a specific desired mRNA. The siRNA denoted as No. 5 in Table 1 was used to reduce the endogenous mRNA level of Annexin II.

Effect of siRNA on Annexin II Gene Expression

The effect of the siRNA on rat Annexin-II gene expression in REF-52 transfected cells was measured by Real-Time-PCR. The expression of GAPDH serves as a reference (control) gene.

TABLE B

| siRNA vector | rAnn-II/GAPDH |
|---|---|
| siLUC | 100 |
| siAnn-II-rB | 17.2 |

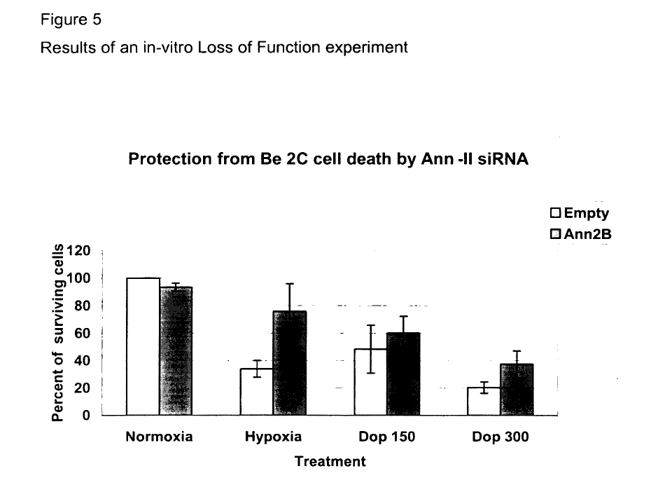

As can be seen, siAnn-II-rB (a vector comprising the Rat Annexin II siRNA depicted in FIG. 5) reduces the expression of rat Annexin II by 82.8%.

This effect was also validated by Western blot analysis: following transfection with the siRNA vactor, the expression of the Annexin II protein is greatly reduced (as measured with a commercially available Annexin II antibody, Santa Cruz).

The effect of the siRNA on Human Annexin-II gene expression in Be2C cells stably transfected with the Annexin II gene was measured by Real-Time-PCR. The expression of Cyclophillin serves as a reference (control) gene.

TABLE C

| siRNA vector | Ann-II/Cyclo |
|---|---|
| siLUC | 100 |
| siAnnII-hB | 24 |

As can be seen, siAnn-II-hB (a vector comprising the Human Annexin II siRNA depicted in FIG. 5) reduces the expression of rat Annexin II by 76%.

This effect was also validated by Western blot analysis: following transfection with the siRNA vactor, the expression of the Annexin II protein is greatly reduced (as measured with a commercially available Annexin II antibody, Santa Cruz).

Loss-of-Function (LOF) Validation of the Importance of Annexin II Activity for Apoptosis

a) In Vivo LOF Results

In vivo delivery of the siRNAs disclosed herein and MCAO experiments (see Example 9) were performed.

Measurement of Annexin-II Expression in the siRNA Treated Brain Tissues:

RNA samples were prepared from brain cortex and striatum and the quality and quantity were assessed by agarose-gell analysis and by O.D. measurement respectively.

A reverse transcriptase reaction was performed and the cDNA product was subjected to quantitative PCR (Real-Time).

For each brain area (cortex, striatum), 4 independent quantitative PCR experiments (29 samples total) were performed. GAPDH was used as an internal control and tested in parallel to Annexin-II.

TABLE D
Summary of quantitative PCR results

| | Brain tissue | | | |
|---|---|---|---|---|
| | Coertex | | Striatum | |
| | Ann-II | | Ann-II | |
| Treatment | expression | Std/Sqrt | expression | Std/Sqrt |
| Normal, Luc, bolus (1, 2, 3) | 0.594 | 0.172 | 0.234 | 0.092 |
| Normal, Ann-II, bolus | 1.171 | 0.526 | 0.420 | 0.095 |
| (4, 5, 6) | | | | |
| MCAO, LUC, bolus (7, 8, 9) | 16.46 | 8.186 | 1.996 | 0.472 |
| MCAO, Ann-II, bolus | 8.75 | 2.735 | 1.182 | 0.133 |
| (10, 11, 12) | | | | |
| Normal, LUC/seline, infusion | 0.393 | 0.05 | 0.167 | 0.033 |
| (13, 14, 15) | | | | |
| Normal, Ann-II, infusion | 0.467 | 0.062 | 0.147 | 0.015 |
| (16, 17, 18) | | | | |
| MCAO, LUC, infusion | 14.84 | 3.51 | 1.246 | 0.265 |
| (19, 20, 21) | | | | |
| MCAO, Ann-II, infusion | 5.62 | 0.387 | 1.082 | 0.383 |
| (22, 23, 24) | | | | |
| Normal, No siRNA, No | 0.274 | 0.008 | 0.077 | 0.012 |
| infusion/bolus | | | | |

Conclusions
1. After the MCAO procedure, the expression of the Annexin-II gene is increased by 10-30 fold. For example, in LUC-infused normal rat (control) the level of Annexin-II expression is 0.393 as compared to 14.84 in LUC-infused MCAO rat.
2. In siRNA-infused rats, a significant reduction of Annexin-II gene expression was achieved In LUC-infused MCAO rats the level of Annexin-II expression was 14.84 as compared to 5.62 in Ann-II infused MCAO rats. In LUC-Bolus MCAO rats the level of Annexin-II expression was 16.46 as compared to 8.75 in Ann-II infused MCAO rats.
3. The effect of the siRNA was observed in the cortex but not in the striatum. Possible explanations:
   i. The siRNA reached the cortex more efficiently than the striatum.
   ii. The MCAO procedure does not elevate the expression of Annexin in striatum (contrary to the cortex).
   iii. As the basal level of Annexin in the striatum is very low, it is difficult to measure siRNA activity.
      b) In Vitro LOF

Stable Be2C cells that expressed the human Annexin II siRNA were treated with 30 uM retinoic acid to induce differentiation. After six days, the cells were fully differentiated and subjected to Dopamine and hypoxia treatment. The viability of the cells was tested using an XTT assay (a cell proliferation assay, based on the ability of metabolically active cells to reduce the tetrazolium salt XTT to orange colored compounds of formazan. The intensity of the dye is proportional to the number of metabolically active (“live”) cells. (Hansen et al, (1989), J. Immunol. Meth. 119, 203-210)). The results presented in FIG. 6 are the summary of 4 independent experiments.

As can be seen from FIG. 6, Annexin II siRNA protects Be2C cells from hypoxia & dopamine mediated cell death.

### Example 4

### Preparation of siRNAs

Using proprietary algorithms and the known sequence of gene Annexin II (SEQ ID NO:1), the sequences of many potential siRNAs were generated. siRNA molecules according to the above specifications were prepared essentially as described herein.

The siRNAs of the present invention can be synthesized by any of the methods which are well-known in the art for synthesis of ribonucleic (or deoxyribonucleic) oligonucleotides. For example, a commercially available machine (available, inter alia, from Applied Biosystems) can be used; the oligonucleotides are prepared according to the sequences disclosed herein. Overlapping pairs of chemically synthesized fragments can be ligated using methods well known in the art (e.g., see U.S. Pat. No. 6,121,426). The strands are synthesized separately and then are annealed to each other in the tube. Then, the double-stranded siRNAs are separated from the single-stranded oligonucleotides that were not annealed (e.g. because of the excess of one of them) by HPLC. In relation to the siRNAs or siRNA fragments of the present invention, two or more such sequences can be synthesized and linked together for use in the present invention.

The siRNA molecules of the invention may be synthesized by procedures known in the art e.g. the procedures as described in Usman et al., 1987, J. Am. Chem. Soc., 109, 7845; Scaringe et al., 1990, Nucleic Acids Res., 18, 5433; Wincott et al., 1995, Nucleic Acids Res. 23, 2677-2684; and Wincott et al., 1997, Methods Mol. Bio., 74, 59, and may make use of common nucleic acid protecting and coupling groups, such as dimethoxytrityl at the 5′-end, and phosphoramidites at the 3′-end. The modified (e.g. 2′-O-methylated) nucleotides and unmodified nucleotides are incorporated as desired.

Alternatively, the nucleic acid molecules of the present invention can be synthesized separately and joined together post-synthetically, for example, by ligation (Moore et al., 1992, Science 256, 9923; Draper et al., International PCT publication No. WO93/23569; Shabarova et al., 1991, Nucleic Acids Research 19, 4247; Bellon et al., 1997, Nucleosides & Nucleotides, 16, 951; Bellon et al., 1997, Bioconjugate Chem. 8, 204), or by hybridization following synthesis and/or deprotection.

The siRNA molecules of the invention can also be synthesized via a tandem synthesis methodology, as described in US patent application publication No. US2004/0019001 (McSwiggen) wherein both siRNA strands are synthesized as a single contiguous oligonucleotide fragment or strand separated by a cleavable linker which is subsequently cleaved to provide separate siRNA fragments or strands that hybridize and permit purification of the siRNA duplex. The linker can be a polynucleotide linker or a non-nucleotide linker. For further information, see PCT publication No. WO 2004/015107 (atugen).

As described above, the siRNAs of Table 1 (below) were constructed such that alternate sugars have 2′-O-methyl modification i.e. alternate nucleotides were thus modified. In these preferred embodiments, in one strand of the siRNA the modified nucleotides were numbers 1,3,5,7,9,11,13,15,17 and 19 and in the opposite strand the modified nucleotides were numbers 2,4,6,8,10,12,14,16 and 18. Thus these siRNAs are blunt-ended 19-mer RNA molecules with alternate 2-0′-methyl modifications as described above. The siRNAs of Tables 2 and 3 (below) are also constructed in this manner; the siRNAs of Table 2 are blunt-ended 19-mer RNA molecules with alternate 2-0′-methyl modifications; the siRNAs of Table 3 are blunt-ended 21-mer RNA molecules with alternate 2-0′-methyl modifications.

Table 1 details various novel siRNA molecules for gene Annexin II which were generated and subsequently synthesized. Several of these siRNAs were tested; for further details, see Example 3. Additional siRNAs can also be tested, for example by transfecting HeLa or Hacat cells with a specific novel siRNA to be tested. Expression of the Annexin II polypeptide can then be determined by Western blotting using an antibody against the Annexin II polypeptide. Any one of the siRNA molecules disclosed herein, and in particular the active molecules detailed in Table 1, are novel and also considered a part of the present invention.

Note that in Table 1 below, the sense strands of siRNAs 1-5 have SEQ ID NOS: 7-11 respectively, and the antisense strands of siRNAs 1-5 have SEQ ID NOS: 12-16 respectively. In Table 2 below, the sense strands of siRNAs 6-107 have SEQ ID NOS: 17-118 respectively, and the antisense strands of siRNAs 6-107 have SEQ ID NOS: 119-220 respectively. In Table 3 below, the sense strands of siRNAs 108-181 have SEQ ID NOS: 221-294 respectively, and the antisense strands of siRNAs 108-181 have SEQ ID NOS: 295-368 respectively.

TABLE 1

| | | | human | human | human | mouse | rat |
|---|---|---|---|---|---|---|---|
| NO. | Sense sequence | AntiSense sequence | 50845387 | 50845385 | 50845389 | 31982484 | 9845233 |
| 1 | CUUUGAUGCUGAGCGGGAU | AUCCCGCUCAGCAUCAAAG | 223-241 | 232-250 | 151-169 | — | — |
| | | | (19/19) | (19/19) | (19/19) | | |
| 2 | GACCGAUCUGGAGAAGGAC | GUCCUUCUCCAGAUCGGUC | 587-601 | 596-610 | 515-529 | 534-548 | 498-516 |
| | | | (15/15) | (15/15) | (15/15) | (15/15) | (19/19) |
| 3 | UCAAGACCAAAGGCGUGGA | UCCACGCCUUUGGUCUUGA | 264-282 | 273-291 192-210 | 211-229 | 179-197 | |
| | | | (18/19) | (18/19) | (18/19) | (18/19) | (19/19) |
| 4 | CCAACCAGGAGCUGCAGGA | UCCUGCAGCUCCUGGUUGG | 534-552 | 543-561 462-480 | 481-496 | 449-467 | |
| | | | (19/19) | (19/19) | (19/19) | (16/16) | (19/19) |
| 5 | CUGAUUGACCAAGAUGCUC | GAGCAUCUUGGUCAAUCAG | 695-713 | 704-722 623-641 | 642-658 | 610-626 | |
| | | | (19/19) | (19/19) | (19/19) | (16/17) | (16/17) |

TABLE 2

| | | | human | human | human | mouse | rat | |
|---|---|---|---|---|---|---|---|---|
| NO. | Sense sIRNA | AntiSense sIRNA | 50845387 | 50845385 | 50845389 | 31982484 | 9845233 | species |

| | | | | | | | | |
|---|---|---|---|---|---|---|---|---|
| 6 | CAAGCUCAGCUUGGAGGGU | ACCCUCCAAGCUGAGCUUG | 154-172 | 163-181 | 82-100 | — | 69-87 | hr |
| | | | (19/19) | (19/19) | (19/19) | | (19/19) | |
| 7 | GGAUGCUUUGAACAUUGAA | UUCAAUGUUCAAAGCAUCC | 238-256 | 247-265 | 166-184 | — | 153-171 | hr |
| | | | (19/19) | (19/19) | (19/19) | | (19/19) | |
| 8 | UUCCUGAACCUGGUUCAGU | ACUGAACCAGGUUCAGGAA | 893-911 | 902-920 | 821-839 | 840-858 | 808-826 | hr |
| | | | (19/19) | (19/19) | (19/19) | (18/19) | (19/19) | |
| 9 | UCCAGCAAGACACUAAGGG | CCCUUAGUGUCUUGCUGGA | 1083-1101 | 1092-1110 | 1011-1029 | 1030-1048 | 998-1016 | hr |
| | | | (19/19) | (19/19) | (19/19) | (18/19) | (19/19) | |
| 10 | CCUGGUUCAGUGCAUUCAG | CUGAAUGCACUGAACCAGG | 901-919 | 910-928 | 829-847 | — | 816-834 | hr |
| | | | (19/19) | (19/19) | (19/19) | | (19/19) | |
| 11 | AUCCUGUGCAAGCUCAGCU | AGCUGAGCUUGCACAGGAU | 146-164 | 155-173 | 74-92 | 93-110 | 61-79 | hr |
| | | | (19/19) | (19/19) | (19/19) | (18/18) | (19/19) | |
| 12 | AAGUGGACAUGUUGAAAAU | AUUUUCAACAUGUCCACUU | 1017-1035 | 1026-1044 | 945-963 | 964-982 | 932-950 | hr |
| | | | (19/19) | (19/19) | (19/19) | (18/19) | (19/19) | |
| 13 | AUUGCCUUCGCCUACCAGA | UCUGGUAGGCGAAGGCAAU | 338-356 | 347-365 | 266-284 | 285-303 | 253-271 | hr |
| | | | (19/19) | (19/19) | (19/19) | (18/19) | (19/19) | |
| 14 | GUUCAGUGCAUUCAGAACA | UGUUCUGAAUGCACUGAAC | 905-923 | 914-932 | 833-851 | — | 820-838 | hr |
| | | | (19/19) | (19/19) | (19/19) | | (19/19) | |
| 15 | CCAAAGAAAUGAACAUUCC | GGAAUGUUCAUUUCUUUGG | 1333-1351 | 1342-1360 | 1261-1279 | — | 1246-1264 | hr |
| | | | (19/19) | (19/19) | (19/19) | | (19/19) | |
| 16 | UCCUGAACCUGGUUCAGUG | CACUGAACCAGGUUCAGGA | 894-912 | 903-921 | 822-840 | 841-859 | 809-827 | hr |
| | | | (19/19) | (19/19) | (19/19) | (18/19) | (19/19) | |
| 17 | UGACUCCAUGAAGGGCAAG | CUUGCCCUUCAUGGAGUCA | 952-970 | 961-979 | 880-898 | 900-917 | 867-885 | hr |
| | | | (19/19) | (19/19) | (19/19) | (18/18) | (19/19) | |
| 18 | UGAAGUGGACAUGUUGAAA | UUUCAACAUGUCCACUUCA | 1015-1033 | 1024-1042 | 943-961 | 962-980 | 930-948 | hr |
| | | | (19/19) | (19/19) | (19/19) | (18/19) | (19/19) | |
| 19 | AGUGAAGUGGACAUGUUGA | UCAACAUGUCCACUUCACU | 1013-1031 | 1022-1040 | 941-959 | 960-974 | 928-946 | hr |
| | | | (19/19) | (19/19) | (19/19) | (15/15) | (19/19) | |
| 20 | UGAAGACACCUGCUCAGUA | UACUGAGCAGGUGUCUUCA | 435-453 | 444-462 | 363-381 | 382-400 | 350-368 | hr |
| | | | (19/19) | (19/19) | (19/19) | (18/19) | (19/19) | |
| 21 | ACCGCAGCAAUGCACAGAG | CUCUGUGCAUUGCUGCGGU | 312-330 | 321-339 | 240-258 | 259-270 | 227-245 | hr |
| | | | (19/19) | (19/19) | (19/19) | (12/12) | (19/19) | |
| 22 | GAAGUGGACAUGUUGAAAA | UUUUCAACAUGUCCACUUC | 1016-1034 | 1025-1043 | 944-962 | 963-981 | 931-949 | hr |
| | | | (19/19) | (19/19) | (19/19) | (18/19) | (19/19) | |
| 23 | GUGCAAGGUCAGCUUGGAG | CUCCAAGCUGAGCUUGCAC | 151-169 | 160-178 | 79-97 | 98-116 | 66-84 | hr |
| | | | (19/19) | (19/19) | (19/19) | (18/19) | (19/19) | |
| 24 | GUGAAGUGGACAUGUUGAA | UUCAACAUGUCCACUUCAC | 1014-1032 | 1023-1041 | 942-960 | 961-979 | 929-947 | hr |
| | | | (19/19) | (19/19) | (19/19) | (18/19) | (19/19) | |
| 25 | GGCUGUAUGACUCCAUGAA | UUCAUGGAGUCAUACAGCC | 945-963 | 954-972 | 873-891 | 892-910 | 860-878 | hr |
| | | | (19/19) | (19/19) | (19/19) | (18/19) | (19/19) | |
| 26 | CGGCUGUAUGACUCCAUGA | UCAUGGAGUCAUACAGCCG | 944-962 | 953-971 | 872-890 | — | 859-877 | hr |
| | | | (19/19) | (19/19) | (19/19) | | (19/19) | |
| 27 | UGAACCUGGUUCAGUGCAU | AUGCACUGAACCAGGUUCA | 897-915 | 906-924 | 825-843 | — | 812-830 | hr |
| | | | (19/19) | (19/19) | (19/19) | | (19/19) | |
| 28 | UGGUUCAGUGCAUUCAGAA | UUCUGAAUGCACUGAACCA | 903-921 | 912-930 | 831-849 | — | 818-836 | hr |
| | | | (19/19) | (19/19) | (19/19) | | (19/19) | |
| 29 | CUGUAUGACUCCAUGAAGG | CCUUCAUGGAGUCAUACAG | 947-965 | 956-974 | 875-893 | 894-912 | 862-880 | hr |
| | | | (19/19) | (19/19) | (19/19) | (18/19) | (19/19) | |
| 30 | ACCUGGUUCAGUGCAUUCA | UGAAUGCACUGAACCAGGU | 900-918 | 909-927 | 828-846 | — | 815-833 | hr |
| | | | (19/19) | (19/19) | (19/19) | | (19/19) | |
| 31 | UGCAAGCUCAGCUUGGAGG | CCUCCAAGCUGAGCUUGCA | 152-170 | 161-179 | 80-98 | 99-117 | 67-85 | hr |
| | | | (19/19) | (19/19) | (19/19) | (18/19) | (19/19) | |
| 32 | CGCAGUGAAGUGGACAUGU | ACAUGUCCACUUCACUGCG | 1010-1028 | 1019-1037 | 938-956 | 957-974 | 925-943 | hr |
| | | | (19/19) | (19/19) | (19/19) | (18/18) | (19/19) | |
| 33 | UUGCCUUCGCCUACCAGAG | CUCUGGUAGGCGAAGGCAA | 339-357 | 348-366 | 267-285 | 286-304 | 254-272 | hr |
| | | | (19/19) | (19/19) | (19/19) | (18/19) | (19/19) | |
| 34 | CCUGAACCUGGUUCAGUGC | GCACUGAACCAGGUUCAGG | 895-913 | 904-922 | 823-841 | 842-860 | 810-828 | hr |
| | | | (19/19) | (19/19) | (19/19) | (18/19) | (19/19) | |
| 35 | CUCAGCUUGGAGGGUGAUC | GAUCACCCUCCAAGCUGAG | 158-176 | 167-185 | 86-104 | 105-123 | 73-91 | hr |
| | | | (19/19) | (19/19) | (19/19) | (18/19) | (19/19) | |
| 36 | GCCAAAGAAAUGAACAUUC | GAAUGUUCAUUUCUUUGGC | 1332-1350 | 1341-1359 | 1260-1278 | — | 1245-1263 | hr |
| | | | (19/19) | (19/19) | (19/19) | | (19/19) | |
| 37 | GCUCAGCUUGGAGGGUGAU | AUCACCCUCCAAGCUGAGC | 157-175 | 166-184 | 85-103 | 104-122 | 72-90 | hr |
| | | | (19/19) | (19/19) | (19/19) | (18/19) | (19/19) | |
| 38 | GAACCUGGUUCAGUGCAUU | AAUGCACUGAACCAGGUUC | 898-916 | 907-925 | 826-844 | — | 813-831 | hr |
| | | | (19/19) | (19/19) | (19/19) | | (19/19) | |
| 39 | GCUGUAUGACUCCAUGAAG | CUUCAUGGAGUCAUACAGC | 946-964 | 955-973 | 874-892 | 893-911 | 861-879 | hr |
| | | | (19/19) | (19/19) | (19/19) | (18/19) | (19/19) | |
| 40 | GCAGUGAAGUGGACAUGUU | AACAUGUCCACUUCACUGC | 1011-1029 | 1020-1038 | 939-957 | 958-974 | 926-944 | hr |
| | | | (19/19) | (19/19) | (19/19) | (17/17) | (19/19) | |
| 41 | GUAUGACUCCAUGAAGGGC | GCCCUUCAUGGAGUCAUAC | 949-967 | 958-976 | 877-895 | 900-914 | 864-882 | hr |
| | | | (19/19) | (19/19) | (19/19) | (15/15) | (19/19) | |
| 42 | GUGCAUUCAGAACAAGCCC | GGGCUUGUUCUGAAUGCAC | 910-928 | 919-937 | 838-856 | 857-875 | 825-843 | hr |
| | | | (19/19) | (19/19) | (19/19) | (18/19) | (19/19) | |
| 43 | UAUGACUCCAUGAAGGGCA | UGCCCUUCAUGGAGUCAUA | 950-968 | 959-977 | 878-896 | 900-915 | 865-883 | hr |
| | | | (19/19) | (19/19) | (19/19) | (16/16) | (19/19) | |
| 44 | GGGAUGCUUUGAACAUUGA | UCAAUGUUCAAAGCAUCCC | 237-255 | 246-264 | 165-183 | — | 152-170 | hr |
| | | | (19/19) | (19/19) | (19/19) | | (19/19) | |
| 45 | UCCUGUGCAAGCUCAGCUU | AAGCUGAGCUUGCACAGGA | 147-165 | 156-174 | 75-93 | 94-110 | 62-80 | hr |
| | | | (19/19) | (19/19) | (19/19) | (17/17) | (19/19) | |
| 46 | GGUUCAGUGCAUUCAGAAC | GUUCUGAAUGCACUGAACC | 904-922 | 913-931 | 832-850 | -819-837 | hr | |
| | | | (19/19) | (19/19) | (19/19) | | (19/19) | |
| 47 | UGCAUUCAGAACAAGCCCC | GGGGCUUGUUCUGAAUGCA | 911-929 | 920-938 | 839-857 | 858-876 | 826-844 | hr |
| | | | (19/19) | (19/19) | (19/19) | (18/19) | (19/19) | |
| 48 | AACCAACCAGGAGCUGCAG | CUGCAGCUCCUGGUUGGUU | 532-550 | 541-559 | 460-478 | 479-496 | 447-465 | hr |
| | | | (19/19) | (19/19) | (19/19) | (18/18) | (19/19) | |
| 49 | CUGUGCAAGCUCAGCUUGG | CCAAGCUGAGCUUGCACAG | 149-167 | 158-176 | 77-95 | 96-110 | 64-82 | hr |
| | | | (19/19) | (19/19) | (19/19) | (15/15) | (19/19) | |
| 50 | UUCAGAACAAGCCCCUGUA | UACAGGGGCUUGUUCUGAA | 915-933 | 924-942 | 843-861 | 864-880 | 830-848 | hr |
| | | | (19/19) | (19/19) | (19/19) | (17/17) | (19/19) | |
| 51 | CAGUGCAUUCAGAACAAGC | GCUUGUUCUGAAUGCACUG | 908-926 | 917-935 | 836-854 | — | 823-841 | hr |
| | | | (19/19) | (19/19) | (19/19) | | (19/19) | |
| 52 | UGUAUGACUCCAUGAAGGG | CCCUUCAUGGAGUCAUACA | 948-966 | 957-975 | 876-894 | 895-913 | 863-881 | hr |
| | | | (19/19) | (19/19) | (19/19) | (18/19) | (19/19) | |
| 53 | GCUUUGAACAUUGAAACAG | CUGUUUCAAUGUUCAAAGC | 242-260 | 251-269 | 170-188 | — | 157-175 | hr |
| | | | (19/19) | (19/19) | (19/19) | | (19/19) | |
| 54 | CUGAACCUGGUUCAGUGCA | UGCACUGAACCAGGUUCAG | 896-914 | 905-923 | 824-842 | 843-861 | 811-829 | hr |
| | | | (19/19) | (19/19) | (19/19) | (18/19) | (19/19) | |
| 55 | CAGUGAAGUGGACAUGUUG | CAACAUGUCCACUUCACUG | 1012-1030 | 1021-1039 | 940-958 | 959-974 | 927-945 | hr |
| | | | (19/19) | (19/19) | (19/19) | (16/16) | (19/19) | |
| 56 | AUCCAGCAAGACACUAAGG | CCUUAGUGUCUUGCUGGAU | 1082-1100 | 1091-1109 | 1010-1028 | 1029-1047 | 997-1015 | hr |
| | | | (19/19) | (19/19) | (19/19) | (18/19) | (19/19) | |
| 57 | AACCUGGUUCAGUGCAUUC | GAAUGCACUGAACCAGGUU | 899-917 | 908-926 | 827-845 | — | 814-832 | hr |
| | | | (19/19) | (19/19) | (19/19) | | (19/19) | |
| 58 | CAAAGAAAUGAACAUUCCA | UGGAAUGUUCAUUUCUUUG | 1334-1352 | 1343-1361 | 1262-1280 | 1279-1297 | 1247-1265 | hr |
| | | | (19/19) | (19/19) | (19/19) | (18/19) | (19/19) | |
| 59 | CAUUCAGAACAAGCCCCUG | CAGGGGCUUGUUCUGAAUG | 913-931 | 922-940 | 841-859 | 864-878 | 828-846 | hr |
| | | | (19/19) | (19/19) | (19/19) | (15/15) | (19/19) | |
| 60 | AAGCUCAGCUUGGAGGGUG | CACCCUCCAAGCUGAGCUU | 155-173 | 164-182 | 83-101 | — | 70-88 | hr |
| | | | (19/19) | (19/19) | (19/19) | | (19/19) | |
| 61 | UCAGCUUGGAGGGUGAUCA | UGAUCACCCUCCAAGCUGA | 159-177 | 168-186 | 87-105 | 106-124 | 74-92 | hr |
| | | | (19/19) | (19/19) | (19/19) | (18/19) | (19/19) | |
| 62 | AGAACCAACCAGGAGCUGC | GCAGCUCCUGGUUGGUUCU | 530-548 | 539-557 | 458-476 | 478-495 | 445-463 | hr |
| | | | (19/19) | (19/19) | (19/19) | (18/18) | (19/19) | |
| 63 | UGCUUUGAACAUUGAAACA | UGUUUCAAUGUUCAAAGCA | 241-259 | 250-268 | 169-187 | — | 156-174 | hr |
| | | | (19/19) | (19/19) | (19/19) | | (19/19) | |
| 64 | AGCUCAGCUUGGAGGGUGA | UCACCCUCCAAGCUGAGCU | 156-174 | 165-183 | 84-102 | — | 71-89 | hr |
| | | | (19/19) | (19/19) | (19/19) | | (19/19) | |
| 65 | AACCGCAGCAAUGCACAGA | UCUGUGCAUUGCUGCGGUU | 311-329 | 320-338 | 239-257 | 258-270 | 226-244 | hr |
| | | | (19/19) | (19/19) | (19/19) | (13/13) | (19/19) | |
| 66 | AGUGCAUUCAGAACAAGCC | GGCUUGUUCUGAAUGCACU | 909-927 | 918-936 | 837-855 | 856-874 | 824-842 | hr |
| | | | (19/19) | (19/19) | (19/19) | (18/19) | (19/19) | |
| 67 | UGUGCAAGCUCAGCUUGGA | UCCAAGCUGAGCUUGCACA | 150-168 | 159-177 | 78-96 | 97-115 | 65-83 | hr |
| | | | (19/19) | (19/19) | (19/19) | (18/19) | (19/19) | |
| 68 | GGUCACCAUUGUCAACAUU | AAUGUUGACAAUGGUGACC | 286-304 | 295-313 | 214-232 | 233-250 | 201-219 | hr |
| | | | (19/19) | (19/19) | (19/19) | (18/18) | (19/19) | |
| 69 | AUGAGUCCAUGAAGGGCAA | UUGCCCUUCAUGGAGUGAU | 951-969 | 960-978 | 879-897 | 900-916 | 866-884 | hr |
| | | | (19/19) | (19/19) | (19/19) | (17/17) | (19/19) | |
| 70 | GCUGCUGUACCUGUGUGGU | ACCACACAGGUACAGOAGO | 1117-1135 | 1126-1144 | 1045-1063 | 1065-1082 | 1032-1050 | hr |
| | | | (19/19) | (19/19) | (19/19) | (18/18) | (19/19) | |
| 71 | UUGAAGACACCUGCUCAGU | ACUGAGCAGGUGUCUUCAA | 434-452 | 443-461 | 362-380 | 381-399 | 349-367 | hr |
| | | | (19/19) | (19/19) | (19/19) | (18/19) | (19/19) | |
| 72 | AUGCUUUGAACAUUGAAAC | GUUUCAAUGUUCAAAGCAU | 240-258 | 249-267 | 168-186 | — | 155-173 | hr |
| | | | (19/19) | (19/19) | (19/19) | | (19/19) | |
| 73 | GCAUUCAGAACAAGCCCCU | AGGGGCUUGUUCUGAAUGC | 912-930 | 921-939 | 840-858 | 859-877 | 827-845 | hr |
| | | | (19/19) | (19/19) | (19/19) | (18/19) | (19/19) | |
| 74 | UUCAGUGCAUUCAGAACAA | UUGUUCUGAAUGCACUGAA | 906-924 | 915-933 | 834-852 | — | 821-839 | hr |
| | | | (19/19) | (19/19) | (19/19) | | (19/19) | |
| 75 | CUGGUUCAGUGCAUUCAGA | UCUGAAUGCACUGAACCAG | 902-920 | 911-929 | 830-848 | — | 817-835 | hr |
| | | | (19/19) | (19/19) | (19/19) | | (19/19) | |
| 76 | CUUUGAACAUUGAAACAGC | GCUGUUUCAAUGUUCAAAG | 243-261 | 252-270 | 171-189 | — | 158-176 | hr |
| | | | (19/19) | (19/19) | (19/19) | | (19/19) | |
| 77 | UCAGUGCAUUCAGAACAAG | CUUGUUCUGAAUGCACUGA | 907-925 | 916-934 | 835-853 | — | 822-840 | hr |
| | | | (19/19) | (19/19) | (19/19) | | (19/19) | |
| 78 | GAUGCUUUGAACAUUGAAA | UUUCAAUGUUCAAAGCAUC | 239-257 | 248-266 | 167-185 | — | 154-172 | hr |
| | | | (19/19) | (19/19) | (19/19) | | (19/19) | |
| 79 | AUUCAGAACAAGCCCCUGU | ACAGGGGCUUGUUCUGAAU | 914-932 | 923-941 | 842-860 | 864-879 | 829-847 | hr |
| | | | (19/19) | (19/19) | (19/19) | (16/16) | (19/19) | |
| 80 | CCUGUGCAAGCUCAGCUUG | CAAGCUGAGCUUGCACAGG | 148-166 | 157-175 | 76-94 | 95-110 | 63-81 | hr |
| | | | (19/19) | (19/19) | (19/19) | (16/16) | (19/19) | |
| 81 | CAAGUGGAUCAGCAUCAUG | CAUGAUGCUGAUCCACUUG | 760-778 | 769-787 | 688-706 | 707-725 | 675-693 | hmr |
| | | | (19/19) | (19/19) | (19/19) | (19/19) | (19/19) | |
| 82 | AAAUCCUGUGCAAGCUCAG | CUGAGCUUGCACAGGAUUU | 144-162 | 153-171 | 72-90 | 91-109 | 59-77 | hmr |
| | | | (19/19) | (19/19) | (19/19) | (19/19) | (19/19) | |
| 83 | CACGAAAUCCUGUGCAAGC | GCUUGCACAGGAUUUCGUG | 140-158 | 149-167 | 68-86 | 87-105 | 55-73 | hmr |
| | | | (19/19) | (19/19) | (19/19) | (19/19) | (19/19) | |
| 84 | UCCAUGAAGGGCAAGGGGA | UCCCCUUGCCCUUCAUGGA | 956-974 | 965-983 | 884-902 | 903-921 | 871-889 | hmr |
| | | | (19/19) | (19/19) | (19/19) | (19/19) | (19/19) | |
| 85 | ACGAAAUCCUGUGCAAGCU | AGCUUGCACAGGAUUUCGU | 141-159 | 150-168 | 69-87 | 88-106 | 56-74 | hmr |
| | | | (19/19) | (19/19) | (19/19) | (19/19) | (19/19) | |
| 86 | GACUCCAUGAAGGGCAAGG | CCUUGCCCUUCAUGGAGUC | 953-971 | 962-980 | 881-899 | 900-918 | 868-886 | hmr |
| | | | (19/19) | (19/19) | (19/19) | (19/19) | (19/19) | |
| 87 | AAGUGGAUCAGCAUCAUGA | UCAUGAUGCUGAUCCACUU | 761-779 | 770-788 | 689-707 | 708-726 | 676-694 | hmr |
| | | | (19/19) | (19/19) | (19/19) | (19/19) | (19/19) | |
| 88 | CCCAAGUGGAUCAGCAUCA | UGAUGCUGAUCCACUUGGG | 758-776 | 767-785 | 686-704 | 705-723 | 673-691 | hmr |
| | | | (19/19) | (19/19) | (19/19) | (19/19) | (19/19) | |
| 89 | GAGGUCACCAUUGUCAACA | UGUUGACAAUGGUGACCUC | 284-302 | 293-311 | 212-230 | 231-249 | 199-217 | hmr |
| | | | (19/19) | (19/19) | (19/19) | (19/19) | (19/19) | |
| 90 | CUGCUGUACCUGUGUGGUG | CACCACACAGGUACAGOAG | 1118-1136 | 1127-1145 | 1046-1064 | 1065-1083 | 1033-1051 | hmr |
| | | | (19/19) | (19/19) | (19/19) | (19/19) | (19/19) | |
| 91 | AGGUCACCAUUGUCAACAU | AUGUUGACAAUGGUGACCU | 285-303 | 294-312 | 213-231 | 232-250 | 200-218 | hmr |
| | | | (19/19) | (19/19) | (19/19) | (19/19) | (19/19) | |
| 92 | AGUGGAUCAGCAUCAUGAC | GUCAUGAUGCUGAUCCACU | 762-780 | 771-789 | 690-708 | 709-727 | 677-695 | hmr |
| | | | (19/19) | (19/19) | (19/19) | (19/19) | (19/19) | |
| 93 | AAUCCUGUGCAAGCUCAGC | GCUGAGCUUGCACAGGAUU | 145-163 | 154-172 | 73-91 | 92-110 | 60-78 | hmr |
| | | | (19/19) | (19/19) | (19/19) | (19/19) | (19/19) | |
| 94 | ACUCCAUGAAGGGCAAGGG | CCCUUGCCCUUCAUGGAGU | 954-972 | 963-981 | 882-900 | 901-919 | 869-887 | hmr |
| | | | (19/19) | (19/19) | (19/19) | (19/19) | (19/19) | |
| 95 | CGAAAUCCUGUGCAAGCUC | GAGCUUGCACAGGAUUUCG | 142-160 | 151-169 | 70-88 | 89-107 | 57-75 | hmr |
| | | | (19/19) | (19/19) | (19/19) | (19/19) | (19/19) | |
| 96 | CCAAGUGGAUCAGCAUCAU | AUGAUGCUGAUCCACUUGG | 759-777 | 768-786 | 687-705 | 706-724 | 674-692 | hmr |
| | | | (19/19) | (19/19) | (19/19) | (19/19) | (19/19) | |
| 97 | GAAAUCCUGUGCAAGCUCA | UGAGCUUGCACAGGAUUUC | 143-161 | 152-170 | 71-89 | 90-108 | 58-76 | hmr |
| | | | (19/19) | (19/19) | (19/19) | (19/19) | (19/19) | |
| 98 | UGCUGUACCUGUGUGGUGG | CCACCACACAGGUACAGCA | 1119-1137 | 1128-1146 | 1047-1065 | 1066-1084 | 1034-1052 | hmr |
| | | | (19/19) | (19/19) | (19/19) | (19/19) | (19/19) | |
| 99 | GAACCAACCAGGAGCUGCA | UGCAGCUCCUGGUUGGUUC | 531-549 | 540-558 | 459-477 | 478-496 | 446-464 | hmr |
| | | | (19/19) | (19/19) | (19/19) | (19/19) | (19/19) | |
| 100 | GCAAGUCCCUGUACUAUUA | UAAUAGUACAGGGACUUGC | 1062-1080 | 1071-1089 | 990-1008 | 1009-1024 | 977-992 | h |
| | | | (19/19) | (19/19) | (19/19) | (16/16) | (15/16) | |
| 101 | GGAUGGCUCUGUCAUUGAU | AUCAAUGACAGAGCCAUCC | 670-688 | 679-697 | 598-616 | — | — | h |
| | | | (19/19) | (19/19) | (19/19) | | | |
| 102 | CCAAGGAGUUGGAAGUGAA | UUCACUUCCAACUCCUUGG | 1350-1368 | 1359-1377 | 1278-1296 | — | — | h |
| | | | (19/19) | (19/19) | (19/19) | | | |
| 103 | GGAGGGUGAUCACUCUACA | UGUAGAGUGAUCACCCUCC | 166-184 | 175-193 | 94-112 | 113-131 | 81-99 | h |
| | | | (19/19) | (19/19) | (19/19) | (18/19) | (18/19) | |
| 104 | GAAGUGAAGUCUAUGAUGU | ACAUCAUAGACUUCACUUC | 1361-1379 | 1370-1388 | 1289-1307 | — | — | h |
| | | | (19/19) | (19/19) | (19/19) | | | |
| 105 | GGAUCAGCAUCAUGACCGA | UCGGUCAUGAUGCUGAUCC | 765-783 | 774-792 | 693-711 | 712-727 | 680-695 | h |
| | | | (19/19) | (19/19) | (19/19) | (16/16) | (16/16) | |
| 106 | GGACUGAGCUGUACAGUAU | AUACUGUACAGCUCAGUGC | 1543-1561 | 1552-1570 | 1471-1489 | — | — | h |
| | | | (19/19) | (19/19) | (19/19) | | | |
| 107 | GAAGACACCUGCUCAGUAU | AUACUGAGCAGGUGUCUUC | 436-454 | 445-463 | 364-382 | 383-401 | 351-368 | h |
| | | | (19/19) | (19/19) | (19/19) | (18/19) | (18/18) | |

TABLE 3

| Num- | | | human | human | human | mouse | rat | spe- |
|---|---|---|---|---|---|---|---|---|
| ber | sense siRNA | AntiSense sIRNA | 50845387 | 50845385 | 50845389 | 31982484 | 9845233 | cies |
| 108 | AAGUGCCAUCGGCGAUGAAGU | AGUUCAUCGCCGAUGGCAGUU | — | — | — | — | 287-307 | man |
| | | | | | | | (21/21) | |
| 109 | CGAAAUCCUGUGCAAGCUCAG | CUGAGCUUGCACAGGAUUUCG | 142-162 | 151-171 | 70-90 | 89-109 | 57-77 | hrm |
| | | | (21/21) | (21/21) | (21/21) | (21/21) | (21/21) | |
| 110 | GAAAUCCUGUGCAAGCUCAGC | GCUGAGCUUGCACAGGAUUUC | 143-163 | 152-172 | 71-91 | 90-110 | 58-78 | hrm |
| | | | (21/21) | (21/21) | (21/21) | (21/21) | (21/21) | |
| 111 | ACUCCAUGAAGGGCAAGGGGA | UCCCCUUGCCCUUCAUGGAGU | 954-974 | 963-983 | 882-902 | 901-921 | 869-889 | hrm |
| | | | (21/21) | (21/21) | (21/21) | (21/21) | (21/21) | |
| 112 | CCCAAGUGGAUCAGCAUCAUG | CAUGAUGCUGAUCCACUUGGG | 758-778 | 767-787 | 686-706 | 705-725 | 673-693 | hrm |
| | | | (21/21) | (21/21) | (21/21) | (21/21) | (21/21) | |
| 113 | CAAGUGGAUCAGCAUCAUGAC | GUCAUGAUGCUGAUCCACUUG | 760-780 | 769-789 | 688-708 | 707-727 | 675-695 | hrm |
| | | | (21/21) | (21/21) | (21/21) | (21/21) | (21/21) | |
| 114 | CACGAAAUCCUGUGCAAGCUC | GAGCUUGCACAGGAUUUCGUG | 140-160 | 149-169 | 68-88 | 87-107 | 55-75 | hrm |
| | | | (21/21) | (21/21) | (21/21) | (21/21) | (21/21) | |
| 115 | CCAAGUGGAUCAGCAUCAUGA | UCAUGAUGCUGAUCCACUUGG | 759-779 | 768-788 | 687-707 | 706-726 | 674-694 | hrm |
| | | | (21/21) | (21/21) | (21/21) | (21/21) | (21/21) | |
| 116 | ACGAAAUCCUGUGCAAGCUCA | UGAGCUUGCACAGGAUUUCGU | 141-161 | 150-170 | 69-89 | 88-108 | 56-76 | hrm |
| | | | (21/21) | (21/21) | (21/21) | (21/21) | (21/21) | |
| 117 | UGACUCCAUGAAGGGCAAGGG | CCCUUGCCCUUCAUGGAGUCA | 952-972 | 961-981 | 880-900 | 900-919 | 867-887 | hr |
| | | | (21/21) | (21/21) | (21/21) | (20/20) | (21/21) | |
| 118 | CUGAACCUGGUUCAGUGCAUU | AAUGCACUGAACCAGGUUCAG | 896-916 | 905-925 | 824-844 | 843-862 | 811-831 | hr |
| | | | (21/21) | (21/21) | (21/21) | (19/20) | (21/21) | |
| 119 | ACCUGGUUCAGUGCAUUCAGA | UCUGAAUGCACUGAACCAGGU | 900-920 | 909-929 | 828-848 | — | 815-835 | hr |
| | | | (21/21) | (21/21) | (21/21) | | (21/21) | |
| 120 | UGAACCUGGUUCAGUGCAUUC | GAAUGCACUGAACCAGGUUCA | 897-917 | 906-926 | 825-845 | — | 812-832 | hr |
| | | | (21/21) | (21/21) | (21/21) | | (21/21) | |
| 121 | GGCUGUAUGACUCCAUGAAGG | CCUUCAUGGAGUCAUACAGCC | 945-965 | 954-974 | 873-893 | 892-912 | 860-880 | hr |
| | | | (21/21) | (21/21) | (21/21) | (20/21) | (21/21) | |
| 122 | UGCAUUCAGAACAAGCCCCUG | CAGGGGCUUGUUCUGAAUGCA | 911-931 | 920-940 | 839-859 | 858-878 | 826-846 | hr |
| | | | (21/21) | (21/21) | (21/21) | (20/21) | (21/21) | |
| 123 | CUGUAUGACUCCAUGAAGGGC | GCCCUUCAUGGAGUCAUACAG | 947-967 | 956-976 | 875-895 | 894-914 | 862-882 | hr |
| | | | (21/21) | (21/21) | (21/21) | (20/21) | (21/21) | |
| 124 | GCAUUCAGAACAAGCCCCUGU | ACAGGGGCUUGUUCUGAAUGC | 912-932 | 921-941 | 840-860 | 859-879 | 827-847 | hr |
| | | | (21/21) | (21/21) | (21/21) | (20/21) | (21/21) | |
| 125 | GAACCUGGUUCAGUGCAUUCA | UGAAUGCACUGAACCAGGUUC | 898-918 | 907-927 | 826-846 | — | 813-833 | hr |
| | | | (21/21) | (21/21) | (21/21) | | (21/21) | |
| 126 | CUGGUUCAGUGCAUUCAGAAC | GUUCUGAAUGCACUGAACCAG | 902-922 | 911-931 | 830-850 | — | 817-837 | hr |
| | | | (21/21) | (21/21) | (21/21) | | (21/21) | |
| 127 | AGCUCAGCUUGGAGGGUGAUC | GAUCACCCUCCAAGCUGAGCU | 156-176 | 165-185 | 84-104 | 103-123 | 71-91 | hr |
| | | | (21/21) | (21/21) | (21/21) | (20/21) | (21/21) | |
| 128 | GUAUGACUCCAUGAAGGGCAA | UUGCCCUUCAUGGAGUCAUAC | 949-969 | 958-978 | 877-897 | 900-916 | 864-884 | hr |
| | | | (21/21) | (21/21) | (21/21) | (17/17) | (21/21) | |
| 129 | GUGCAUUCAGAACAAGCCCCU | AGGGGCUUGUUCUGAAUGCAC | 910-930 | 919-939 | 838-858 | 857-877 | 825-845 | hr |
| | | | (21/21) | (21/21) | (21/21) | (20/21) | (21/21) | |
| 130 | GUUCAGUGCAUUCAGAACAAG | CUUGUUCUGAAUGCACUGAAC | 905-925 | 914-934 | 833-853 | — | 820-840 | hr |
| | | | (21/21) | (21/21) | (21/21) | | (21/21) | |
| 131 | AAGCUCAGCUUGGAGGGUGAU | AUCACCCUCCAAGCUGAGCUU | 155-175 | 164-184 | 83-103 | 102-122 | 70-90 | hr |
| | | | (21/21) | (21/21) | (21/21) | (20/21) | (21/21) | |
| 132 | AGUGCAUUCAGAACAAGCCCC | GGGGCUUGUUCUGAAUGCACU | 909-929 | 918-938 | 837-857 | 856-876 | 824-844 | hr |
| | | | (21/21) | (21/21) | (21/21) | (20/21) | (21/21) | |
| 133 | AUCCUGUGCAAGCUCAGCUUG | CAAGCUGAGCUUGCACAGGAU | 146-166 | 155-175 | 74-94 | 93-110 | 61-81 | hr |
| | | | (21/21) | (21/21) | (21/21) | (18/18) | (21/21) | |
| 134 | AGAACCAACCAGGAGCUGCAG | CUGCAGCUCCUGGUUGGUUCU | 530-550 | 539-559 | 458-478 | 478-496 | 445-465 | hr |
| | | | (21/21) | (21/21) | (21/21) | (19/19) | (21/21) | |
| 135 | UCCUGUGCAAGCUCAGCUUGG | CCAAGCUGAGCUUGCACAGGA | 147-167 | 156-176 | 75-95 | 94-110 | 62-82 | hr |
| | | | (21/21) | (21/21) | (21/21) | (17/17) | (21/21) | |
| 136 | CCUGUGCAAGCUCAGCUUGGA | UCCAAGCUGAGCUUGCACAGG | 148-168 | 157-177 | 76-98 | 95-115 | 63-83 | hr |
| | | | (21/21) | (21/21) | (21/21) | (20/21) | (21/21) | |
| 137 | UUCCUGAAGCUGGUUCAGUGC | GCACUGAAGCAGGUUCAGGAA | 893-913 | 902-922 | 821-841 | 840-860 | 808-828 | hr |
| | | | (21/21) | (21/21) | (21/21) | (20/21) | (21/21) | |
| 138 | GCAGUGAAGUGGACAUGUUGA | UCAACAUGUCCACUUCACUGC | 1011-1031 | 1020-1040 | 939-959 | 958-974 | 926-946 | hr |
| | | | (21/21) | (21/21) | (21/21) | (17/17) | (21/21) | |
| 139 | AAUCCUGUGCAAGCUCAGCUU | AAGCUGAGCUUGCACAGGAUU | 145-165 | 154-174 | 73-93 | 92-110 | 60-80 | hr |
| | | | (21/21) | (21/21) | (21/21) | (19/19) | (21/21) | |
| 140 | UGCUUUGAACAUUGAAACAGC | GCUGUUUCAAUGUUCAAAGCA | 241-261 | 250-270 | 169-189 | — | 156-176 | hr |
| | | | (21/21) | (21/21) | (21/21) | | (21/21) | |
| 141 | UGCAAGCUCAGCUUGGAGGGU | ACCCUCCAAGCUGAGCUUGCA | 152-172 | 161-181 | 80-100 | 99-119 | 67-87 | hr |
| | | | (21/21) | (21/21) | (21/21) | (20/21) | (21/21) | |
| 142 | UCCUGAACCUGGUUCAGUGCA | UGCACUGAACCAGGUUCAGGA | 894-914 | 903-923 | 822-842 | 841-861 | 809-829 | hr |
| | | | (21/21) | (21/21) | (21/21) | (20/21) | (21/21) | |
| 143 | CAGUGAAGUGGACAUGUUGAA | UUCAACAUGUCGACUUCACUG | 1012-1032 | 1021-1041 | 940-960 | 959-979 | 927-947 | hr |
| | | | (21/21) | (21/21) | (21/21) | (20/21) | (21/21) | |
| 144 | UGAAGUGGACAUGUUGAAAAU | AUUUUCAACAUGUCCACUUCA | 1015-1035 | 1024-1044 | 943-963 | 962-982 | 930-950 | hr |
| | | | (21/21) | (21/21) | (21/21) | (20/21) | (21/21) | |
| 145 | UUCAGUGCAUUCAGAACAAGC | GCUUGUUCUGAAUGCACUGAA | 906-926 | 915-935 | 834-854 | — | 821-841 | hr |
| | | | (21/21) | (21/21) | (21/21) | | (21/21) | |
| 146 | UGGUUCAGUGCAUUCAGAACA | UGUUCUGAAUGCACUGAACCA | 903-923 | 912-932 | 831-851 | — | 818-838 | hr |
| | | | (21/21) | (21/21) | (21/21) | | (21/21) | |
| 147 | UCAGUGCAUUCAGAACAAGCC | GGCUUGUUCUGAAUGCACUGA | 907-927 | 916-936 | 835-855 | 855-874 | 822-842 | hr |
| | | | (21/21) | (21/21) | (21/21) | (19/20) | (21/21) | |
| 148 | GUGAAGUGGACAUGUUGAAAA | UUUUCAACAUGUCCACUUCAC | 1014-1034 | 1023-1043 | 942-962 | 961-981 | 929-949 | hr |
| | | | (21/21) | (21/21) | (21/21) | (20/21) | (21/21) | |
| 149 | CGGCUGUAUGACUCCAUGAAG | CUUCAUGGAGUCAUACAGCCG | 944-964 | 953-973 | 872-892 | 891-911 | 859-879 | hr |
| | | | (21/21) | (21/21) | (21/21) | (20/21) | (21/21) | |
| 150 | GAUGCUUUGAACAUUGAAACA | UGUUUCAAUGUUCAAAGCAUC | 239-259 | 248-268 | 167-187 | — | 154-174 | hr |
| | | | (21/21) | (21/21) | (21/21) | | (21/21) | |
| 151 | AUGACUCCAUGAAGGGCAAGG | CCUUGCCCUUCAUGGAGUCAU | 951-971 | 960-980 | 879-899 | 900-918 | 866-886 | hr |
| | | | (21/21) | (21/21) | (21/21) | (19/19) | (21/21) | |
| 152 | CAGUGCAUUCAGAACAAGCCC | GGGCUUGUUCUGAAUGCACUG | 908-928 | 917-937 | 836-856 | 855-875 | 823-843 | hr |
| | | | (21/21) | (21/21) | (21/21) | (20/21) | (21/21) | |
| 153 | UAUGACUCCAUGAAGGGCAAG | CUUGCCCUUCAUGGAGUCAUA | 950-970 | 959-979 | 878-898 | 900-917 | 865-885 | hr |
| | | | (21/21) | (21/21) | (21/21) | (18/18) | (21/21) | |
| 154 | GGAUGCUUUGAACAUUGAAAC | GUUUCAAUGUUCAAAGCAUCC | 238-258 | 247-267 | 166-186 | — | 153-173 | hr |
| | | | (21/21) | (21/21) | (21/21) | | (21/21) | |
| 155 | GAGGUCACCAUUGUCAACAUU | AAUGUUGACAAUGGUGACCUC | 284-304 | 293-313 | 212-232 | 231-250 | 199-219 | hr |
| | | | (21/21) | (21/21) | (21/21) | (20/20) | (21/21) | |
| 156 | AACCAACCAGGAGCUGCAGGA | UCCUGCAGCUCCUGGUUGGUU | 532-552 | 541-561 | 460-480 | 479-496 | 447-467 | hr |
| | | | (21/21) | (21/21) | (21/21) | (18/18) | (21/21) | |
| 157 | GGUUCAGUGCAUUCAGAACAA | UUGUUCUGAAUGCACUGAACC | 904-924 | 913-933 | 832-852 | — | 819-839 | hr |
| | | | (21/21) | (21/21) | (21/21) | | (21/21) | |
| 158 | GGGAUGCUUUGAACAUUGAAA | UUUCAAUGUUCAAAGCAUCCC | 237-257 | 246-266 | 165-185 | — | 152-172 | hr |
| | | | (21/21) | (21/21) | (21/21) | | (21/21) | |
| 159 | CAUUCAGAACAAGCCCCUGUA | UACAGGGGCUUGUUCUGAAUG | 913-933 | 922-942 | 841-861 | 864-880 | 828-848 | hr |
| | | | (21/21) | (21/21) | (21/21) | (17/17) | (21/21) | |
| 160 | GCUGUAUGACUCCAUGAAGGG | CCCUUCAUGGAGUCAUACAGC | 946-966 | 955-975 | 874-894 | 893-913 | 861-881 | hr |
| | | | (21/21) | (21/21) | (21/21) | (20/21) | (21/21) | |
| 161 | UGUGCAAGCUCAGCUUGGAGG | CCUCCAAGCUGAGCUUGCACA | 150-170 | 159-179 | 78-98 | 97-117 | 65-85 | hr |
| | | | (21/21) | (21/21) | (21/21) | (20/21) | (21/21) | |
| 162 | CGCAGUGAAGUGGACAUGUUG | CAACAUGUCCACUUCACUGCG | 1010-1030 | 1019-1039 | 938-958 | 957-974 | 925-945 | hr |
| | | | (21/21) | (21/21) | (21/21) | (18/18) | (21/21) | |
| 163 | CCUGAACCUGGUUCAGUGCAU | AUGCACUGAACCAGGUUCAGG | 895-915 | 904-924 | 823-843 | 842-862 | 810-830 | hr |
| | | | (21/21) | (21/21) | (21/21) | (20/21) | (21/21) | |
| 164 | CCUGGUUCAGUGCAUUCAGAA | UUCUGAAUGCACUGAACCAGG | 901-921 | 910-930 | 829-849 | — | 816-836 | hr |
| | | | (21/21) | (21/21) | (21/21) | | (21/21) | |
| 165 | GCCAAAGAAAUGAACAUUCCA | UGGAAUGUUCAUUUCUUUGGC | 1332-1352 | 1341-1361 | 1260-1280 | 1277-1297 | 1245-1265 | hr |
| | | | (21/21) | (21/21) | (21/21) | (20/21) | (21/21) | |
| 166 | GCUCAGCUUGGAGGGUGAUCA | UGAUCACCCUCCAAGCUGAGC | 157-177 | 166-186 | 85-105 | 104-124 | 72-92 | hr |
| | | | (21/21) | (21/21) | (21/21) | (20/21) | (21/21) | |
| 167 | UGUAUGACUCCAUGAAGGGCA | UGCCCUUCAUGGAGUCAUAGA | 948-968 | 957-977 | 876-896 | 895-915 | 863-883 | hr |
| | | | (21/21) | (21/21) | (21/21) | (20/21) | (21/21) | |
| 168 | AACCUGGUUCAGUGCAUUCAG | CUGAAUGCACUGAACCAGGUU | 899-919 | 908-928 | 827-847 | — | 814-834 | hr |
| | | | (21/21) | (21/21) | (21/21) | | (21/21) | |
| 169 | CUGUGCAAGCUCAGCUUGGAG | CUCCAAGCUGAGCUUGCACAG | 149-169 | 158-178 | 77-97 | 96-116 | 64-84 | hr |
| | | | (21/21) | (21/21) | (21/21) | (20/21) (21/21) | | |
| 170 | CAAGCUCAGCUUGGAGGGUGA | UCACCCUCCAAGCUGAGCUUG | 154-174 | 163-183 | 82-102 | — | 69-89 | hr |
| | | | (21/21) | (21/21) | (21/21) | | (21/21) | |
| 171 | AAAUCCUGUGCAAGCUCAGCU | AGCUGAGCUUGCACAGGAUUU | 144-164 | 153-173 | 72-92 | 91-110 | 59-79 | hr |
| | | | (21/21) | (21/21) | (21/21) | (20/20) | (21/21) | |
| 172 | AUGCUUUGAACAUUGAAACAG | CUGUUUCAAUGUUCAAAGCAU | 240-260 | 249-269 | 168-188 | — | 155-175 | hr |
| | | | (21/21) | (21/21) | (21/21) | | (21/21) | |
| 173 | AGUGAAGUGGACAUGUUGAAA | UUUCAACAUGUCCACUUCACU | 1013-1033 | 1022-1042 | 941-961 | 960-980 | 928-948 | hr |
| | | | (21/21) | (21/21) | (21/21) | (20/21) | (21/21) | |
| 174 | GGAUGGCUCUGUCAUUGAUUA | UAAUCAAUGACAGAGCCAUCC | 670-690 | 679-699 | 598-618 | — | — | h |
| | | | (21/21) | (21/21) | (21/21) | | | |
| 175 | CCAAAGAAAUGAACAUUCCAA | UUGGAAUGUUCAUUUCUUUGG | 1333-1353 | 1342-1362 | 1261-1281 | 1278-1297 | 1246-1265 | h |
| | | | (21/21) | (21/21) | (21/21) | (19/20) | (20/20) | |
| 176 | GAGAUAAGGUCCUGAUCAGAA | UUCUGAUCAGGACCUUAUCUC | 978-998 | 987-1007 | 906-926 | 925-945 | 893-913 | h |
| | | | (21/21) | (21/21) | (21/21) | (19/21) | (19/21) | |
| 177 | GCAAGUCCCUGUACUAUUAUA | UAUAAUAGUACAGGGACUUGC | 1062-1082 | 1071-1091 | 990-1010 | 1009-1024 | 977-992 | h |
| | | | (21/21) | (21/21) | (21/21) | (16/16) | (15/16) | |
| 178 | CGAGGACUCUCUCAUUGAGAU | AUCUCAAUGAGAGAGUCCUCG | 499-519 | 508-528 | 427-447 | 446-466 | — | h |
| | | | (21/21) | (21/21) | (21/21) | (20/21) | | |
| 179 | CCAAGGAGUUGGAAGUGAAGU | ACUUCACUUCCAACUCCUUGG | 1350-1370 | 1359-1379 | 1278-1298 | — | — | h |
| | | | (21/21) | (21/21) | (21/21) | | | |
| 180 | GAAGUGAAGUCUAUGAUGUGA | UCACAUCAUAGACUUCACUUC | 1361-1381 | 1370-1390 | 1289-1309 | — | — | h |
| | | | (21/21) | (21/21) | (21/21) | | | |
| 181 | GAAGACACCUGCUCAGUAUGA | UCAUACUGAGCAGGUGUCUUC | 436-456 | 445-465 | 364-384 | 383-403 | 351-368 | h |
| | | | (21/21) | (21/21) | (21/21) | (20/21) | (18/18) | |

### Example 5

### Preparation of Anti-Annexin II Antibodies

Antibodies which bind to Annexin II may be prepared using an intact polypeptide or fragments containing smaller polypeptides as the immunizing antigen. For example, it may be desirable to produce antibodies that specifically bind to the N- or C-terminal or any other suitable domains of the Annexin II. The polypeptide used to immunize an animal can be derived from translated cDNA or chemical synthesis which can be conjugated to a carrier protein, if desired. Such commonly used carriers which are chemically coupled to the polypeptide include keyhole limpet hemocyanin (KLH), thyroglobulin, bovine serum albumin (BSA) and tetanus toxoid. The coupled polypeptide is then used to immunize the animal.

If desired, polyclonal or monoclonal antibodies can be further purified, for example by binding to and elution from a matrix to which the polypeptide or a peptide to which the antibodies were raised is bound. Those skilled in the art know various techniques common in immunology for purification and/or concentration of polyclonal as well as monoclonal antibodies (Coligan et al, Unit 9, Current Protocols in Immunology, Wiley Interscience, 1994).

Methods for making antibodies of all types, including fragments, are known in the art (See for example, Harlow and Lane, Antibodies: A Laboratory Manual, Cold Spring Harbor Laboratory, New York (1988)). Methods of immunization, including all necessary steps of preparing the immunogen in a suitable adjuvant, determining antibody binding, isolation of antibodies, methods for obtaining monoclonal antibodies, and humanization of monoclonal antibodies are all known to the skilled artisan

The antibodies may be humanized antibodies or human antibodies. Antibodies can be humanized using a variety of techniques known in the art including CDR-grafting (EP239,400: PCT publication WO91/09967; U.S. Pat. Nos. 5,225,539; 5,530,101; and 5,585,089, veneering or resurfacing (EP 592,106; EP 519,596; Padlan, Molecular Immunology 28(4/5):489-498 (1991); Studnicka et al., Protein Engineering 7(6):805-814 (1994); Roguska et al., PNAS 91:969-973 (1994)), and chain shuffling (U.S. Pat. No. 5,565,332).

The monoclonal antibodies as defined include antibodies derived from one species (such as murine, rabbit, goat, rat, human, etc.) as well as antibodies derived from two (or more) species, such as chimeric and humanized antibodies.

Completely human antibodies are particularly desirable for therapeutic treatment of human patients. Human antibodies can be made by a variety of methods known in the art including phage display methods using antibody libraries derived from human immunoglobulin sequences. See also U.S. Pat. Nos. 4,444,887 and 4,716,111; and PCT publications WO 98/46645, WO 98/50433, WO 98/24893, WO 98/16654, WO 96/34096, WO 96/33735, and WO 91/10741, each of which is incorporated herein by reference in its entirety.

Additional information regarding all types of antibodies, including humanized antibodies, human antibodies and antibody fragments can be found in WO 01/05998, which is incorporated herein by reference in its entirety.

### Example 6

### Preparation of Polypeptides

Polypeptides may be produced via several methods, for example:

1) Synthetically:

Synthetic polypeptides can be made using a commercially available machine, using the known sequence of Annexin II.

2) Recombinant Methods:

A preferred method of making the Annexin II polypeptides is to clone a polynucleotide comprising the cDNA of the Annexin II gene into an expression vector and culture the cell harboring the vector so as to express the encoded polypeptide, and then purify the resulting polypeptide, all performed using methods known in the art as described in, for example, Marshak et al., “Strategies for Protein Purification and Characterization. A laboratory course manual.” CSHL Press (1996). (in addition, see Bibl Haematol. 1965; 23:1165-74 Appl Microbiol. 1967 July; 15(4):851-6; Can J. Biochem. 1968 May; 46(5):441-4; Biochemistry. 1968 July; 7(7):2574-80; Arch Biochem Biophys. 1968 Sep. 10; 126(3):746-72; Biochem Biophys Res Commun. 1970 Feb. 20; 38(4):825-30).).

The expression vector can include a promoter for controlling transcription of the heterologous material and can be either a constitutive or inducible promoter to allow selective transcription. Enhancers that can be required to obtain necessary transcription levels can optionally be included. The expression vehicle can also include a selection gene.

Vectors can be introduced into cells or tissues by any one of a variety of methods known within the art. Such methods can be found generally described in Sambrook et al., Molecular Cloning: A Laboratory Manual, Cold Springs Harbor Laboratory, New York (1989, 1992), in Ausubel et al., Current Protocols in Molecular Biology, John Wiley and Sons, Baltimore, Md. (1989), Vega et al., Gene Targeting, CRC Press, Ann Arbor, Mich. (1995), Vectors: A Survey of Molecular Cloning Vectors and Their Uses, Butterworths, Boston Mass. (1988) and Gilboa et al. (1986).

3) Purification from Natural Sources:

Annexin II can be purified from natural sources (such as tissues) using many methods known to one of ordinary skill in the art, such as for example: immuno-precipitation with anti-Annexin II antibody, or matrix-bound affinity chromatography with any molecule known to bind Annexin II.

Protein purification is practiced as is known in the art as described in, for example, Marshak et al., “Strategies for Protein Purification and Characterization. A laboratory course manual.” CSHL Press (1996).

### Example 7

### Preparation of Polynucleotides

The polynucleotides of the present invention can be synthesized by any of the methods that are well-known in the art for synthesis of ribonucleic or deoxyribonucleic oligonucleotides. Such synthesis is, among others, described in Beaucage S. L. and Iyer R. P., Tetrahedron 1992; 48: 2223-2311, Beaucage S. L. and Iyer R. P., Tetrahedron 1993; 49: 6123-6194 and Caruthers M. H. et. al., Methods Enzymol. 1987; 154: 287-313, the synthesis of thioates is, among others, described in Eckstein F., Annu. Rev. Biochem. 1985; 54: 367-402, the synthesis of RNA molecules is described in Sproat B., in Humana Press 2005 Edited by Herdewijn P.; Kap. 2: 17-31 and respective downstream processes are, among others, described in Pingoud A. et. al., in IRL Press 1989 Edited by Oliver R. W. A.; Kap. 7: 183-208 and Sproat B., in Humana Press 2005 Edited by Herdewijn P.; Kap. 2: 17-31 (supra).

Other synthetic procedures are known in the art e.g. the procedures as described in Usman et al., 1987, J. Am. Chem. Soc., 109, 7845; Scaringe et al., 1990, Nucleic Acids Res., 18, 5433; Wincott et al., 1995, Nucleic Acids Res. 23, 2677-2684; and Wincott et al., 1997, Methods Mol. Bio., 74, 59, and these procedures may make use of common nucleic acid protecting and coupling groups, such as dimethoxytrityl at the 5′-end, and phosphoramidites at the 3′-end. The modified (e.g. 2′-O-methylated) nucleotides and unmodified nucleotides are incorporated as desired.

The oligonucleotides of the present invention can be synthesized separately and joined together post-synthetically, for example, by ligation (Moore et al., 1992, Science 256, 9923; Draper et al., International PCT publication No. WO93/23569; Shabarova et al., 1991, Nucleic Acids Research 19, 4247; Bellon et al., 1997, Nucleosides & Nucleotides, 16, 951; Bellon et al., 1997, Bioconjugate Chem. 8, 204), or by hybridization following synthesis and/or deprotection.

It is noted that a commercially available machine (available, inter alia, from Applied Biosystems) can be used; the oligonucleotides are prepared according to the sequences disclosed herein. Overlapping pairs of chemically synthesized fragments can be ligated using methods well known in the art (e.g., see U.S. Pat. No. 6,121,426). The strands are synthesized separately and then are annealed to each other in the tube. Then, the double-stranded siRNAs are separated from the single-stranded oligonucleotides that were not annealed (e.g. because of the excess of one of them) by HPLC. In relation to the siRNAs or siRNA fragments of the present invention, two or more such sequences can be synthesized and linked together for use in the present invention.

The compounds of the invention can also be synthesized via a tandem synthesis methodology, as described in US patent application publication No. US2004/0019001 (McSwiggen), wherein both siRNA strands are synthesized as a single contiguous oligonucleotide fragment or strand separated by a cleavable linker which is subsequently cleaved to provide separate siRNA fragments or strands that hybridize and permit purification of the siRNA duplex. The linker can be a polynucleotide linker or a non-nucleotide linker.

Another means of isolating a polynucleotide is to obtain a natural or artificially designed DNA fragment based on that sequence. This DNA fragment is labeled by means of suitable labeling systems which are well known to those of skill in the art; see, e.g., Davis et al. (1986). The fragment is then used as a probe to screen a lambda phage cDNA library or a plasmid cDNA library using methods well known in the art; see, generally, Sambrook et al., Molecular Cloning: A Laboratory Manual, Cold Spring Harbor Laboratory Press, New York (1989), in Ausubel et al., Current Protocols in Molecular Biology, John Wiley and Sons, Baltimore, Md. (1989),

Colonies can be identified which contain clones related to the cDNA probe and these clones can be purified by known methods. The ends of the newly purified clones are then sequenced to identify full-length sequences. Complete sequencing of full-length clones is performed by enzymatic digestion or primer walking. A similar screening and clone selection approach can be applied to clones from a genomic DNA library.

The polynucleotides disclosed herein can be used inter alia as a probe for diagnostic work. They can be used to diagnose cells which have undergone stroke, neurotoxic stress or TBI, whereby said polynucleotide sequence is over-expressed and there are, thus, high levels of mRNA gene transcripts. In addition, it can be used to diagnose cells which have undergone a cancerous transformation, in which case the aforementioned polynucleotide would be under-expressed (and its level can be compared to the level in a normal subject for the purpose of diagnosis).

### Example 8

### Pharmacology and Drug Delivery

The compounds or pharmaceutical compositions of the present invention are administered and dosed in accordance with good medical practice, taking into account the clinical condition of the individual patient, the disease to be treated, the site and method of administration, scheduling of administration, patient age, sex, body weight and other factors known to medical practitioners.

The pharmaceutically “effective amount” for purposes herein is thus determined by such considerations as are known in the art. The amount must be effective to achieve improvement including but not limited to improved survival rate or more rapid recovery, or improvement or elimination of symptoms and other indicators as are selected as appropriate measures by those skilled in the art.

The treatment generally has a length proportional to the length of the disease process and drug effectiveness and the patient species being treated. It is noted that humans are treated generally longer than the mice or other experimental animals exemplified herein.

The compounds of the present invention can be administered by any of the conventional routes of administration. It should be noted that the compound can be administered as the compound or as pharmaceutically acceptable salt and can be administered alone or as an active ingredient in combination with pharmaceutically acceptable carriers, solvents, diluents, excipients, adjuvants and vehicles. The compounds can be administered orally, subcutaneously or parenterally including intravenous, intraarterial, intramuscular, intraperitoneally, and intranasal administration as well as intrathecal and infusion techniques. Implants of the compounds are also useful. Liquid forms may be prepared for injection, the term including subcutaneous, transdermal, intravenous, intramuscular, intrathecal, and other parental routes of administration. The liquid compositions include aqueous solutions, with and without organic cosolvents, aqueous or oil suspensions, emulsions with edible oils, as well as similar pharmaceutical vehicles. In addition, under certain circumstances the compositions for use in the novel treatments of the present invention may be formed as aerosols, for intranasal and like administration. The patient being treated is a warm-blooded animal and, in particular, mammals including man. The pharmaceutically acceptable carriers, solvents, diluents, excipients, adjuvants and vehicles as well as implant carriers generally refer to inert, non-toxic solid or liquid fillers, diluents or encapsulating material not reacting with the active ingredients of the invention.

When administering the compound of the present invention parenterally, it is generally formulated in a unit dosage injectable form (solution, suspension, emulsion). The pharmaceutical formulations suitable for injection include sterile aqueous solutions or dispersions and sterile powders for reconstitution into sterile injectable solutions or dispersions. The carrier can be a solvent or dispersing medium containing, for example, water, ethanol, polyol (for example, glycerol, propylene glycol, liquid polyethylene glycol, and the like), suitable mixtures thereof, and vegetable oils.

Proper fluidity can be maintained, for example, by the use of a coating such as lecithin, by the maintenance of the required particle size in the case of dispersion and by the use of surfactants. Nonaqueous vehicles such a cottonseed oil, sesame oil, olive oil, soybean oil, corn oil, sunflower oil, or peanut oil and esters, such as isopropyl myristate, can also be used as solvent systems for compound compositions. Additionally, various additives which enhance the stability, sterility, and isotonicity of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, sorbic acid, and the like. In many cases, it is desirable to include isotonic agents, for example, sugars, sodium chloride, and the like. Prolonged absorption of the injectable pharmaceutical form can be brought about by the use of agents delaying absorption, for example, aluminum monostearate and gelatin. According to the present invention, however, any vehicle, diluent, or additive used have to be compatible with the compounds.

Sterile injectable solutions can be prepared by incorporating the compounds utilized in practicing the present invention in the required amount of the appropriate solvent with various of the other ingredients, as desired.

A pharmacological formulation of the present invention can be administered to the patient in an injectable formulation containing any compatible carrier, such as various vehicle, adjuvants, additives, and diluents; or the compounds utilized in the present invention can be administered parenterally to the patient in the form of slow-release subcutaneous implants or targeted delivery systems such as monoclonal antibodies, vectored delivery, iontophoretic, polymer matrices, liposomes, and microspheres. Examples of delivery systems useful in the present invention include U.S. Pat. Nos. 5,225,182; 5,169,383; 5,167,616; 4,959,217; 4,925,678; 4,487,603; 4,486,194; 4,447,233; 4,447,224; 4,439,196; and 4,475,196. Many other such implants, delivery systems, and modules are well known to those skilled in the art.

A pharmacological formulation of the compound utilized in the present invention can be administered orally to the patient. Conventional methods such as administering the compound in tablets, suspensions, solutions, emulsions, capsules, powders, syrups and the like are usable. Known techniques which deliver it orally or intravenously and retain the biological activity are preferred. In one embodiment, the compound of the present invention can be administered initially by intravenous injection to bring blood levels to a suitable level. The patient's levels are then maintained by an oral dosage form, although other forms of administration, dependent upon the patient's condition and as indicated above, can be used.

In general, the active dose of compound for humans is in the range of from 1 ng/kg to about 20-100 mg/kg body weight per day, preferably about 0.01 mg to about 2-10 mg/kg body weight per day, in a regimen of one dose per day or twice or three or more times per day for a period of 1-2 weeks or longer, preferably for 24-to 48 hrs or by continuous infusion during a period of 1-2 weeks or longer. Treatment for many years or even lifetime treatment is also envisaged for some of the indications disclosed herein.

It will be appreciated that the most appropriate administration of the pharmaceutical compositions of the present invention will depend on the type of injury or disease being treated. Thus, the treatment of an acute event will necessitate systemic administration of the active composition comparatively rapidly after induction of the injury. On the other hand, treatment (diminution) of chronic degenerative damage may necessitate a sustained dosage regimen.

Delivery of Annexin II Inhibitors into the Brain

Delivery of compounds into the brain can be accomplished by several methods such as, inter alia, neurosurgical implants, blood-brain barrier disruption, lipid mediated transport, carrier mediated influx or efflux, plasma protein-mediated transport, receptor-mediated transcytosis, absorptive-mediated transcytosis, neuropeptide transport at the blood-brain barrier, and genetically engineering “Trojan horses” for drug targeting. The above methods are performed for example as described in “Brain Drug Targeting: the future of brain drug development”, W. M. Pardridge, Cambridge University Press, Cambridge, UK (2001).

### Example 9

### Experimental Models

CNS injury—The potential of the use of an Annexin II inhibitor for treating CNS injury is evaluated in animal models. The models represent varying levels of complexity, by comparison of control animals to the inhibitor treated animals. The efficacy of such treatment is evaluated in terms of clinical outcome, neurological deficit, dose-response and therapeutic window. Test animals are treated with a Annexin II inhibitor intravenously or subcutanously or per os. Control animals are treated with buffer or pharmaceutical vehicle only. Models used may be selected from the following:
1. Closed Head Injury (CHI)— Experimental TBI produces a series of events contributing to neurological and neurometabolic cascades, which are related to the degree and extent of behavioral deficits. CHI is induced under anesthesia, while a weight is allowed to free-fall from a prefixed height (Chen et al, J. Neurotrauma 13, 557, 1996) over the exposed skull covering the left hemisphere in the midcoronal plane.
2. Transient middle cerebral artery occlusion (MCAO)— a 90 to 120 minutes transient focal ischemia is performed in adult, male Sprague Dawley rats, 300-370 gr. The method employed is the intraluminal suture MCAO (Longa et al., Stroke, 30, 84, 1989, and Dogan et al., J. Neurochem. 72, 765, 1999). Briefly, under halothane anesthesia, a 3-0-nylon suture material coated with Poly-L-Lysine is inserted into the right internal carotid artery (ICA) through a hole in the external carotid artery. The nylon thread is pushed into the ICA to the right MCA origin (20-23 mm). 90-120 minutes later the thread is pulled off, the animal is closed and allowed to recover.
3. Permanent middle cerebral artery occlusion (MCAO)— occlusion is permanent, unilateral-induced by electrocoagulation of MCA. Both methods lead to focal brain ischemia of the ipsilateral side of the brain cortex leaving the contralateral side intact (control). The left MCA is exposed via a temporal craniectomy, as described for rats by Tamura A. et al., J Cereb Blood Flow Metab. 1981; 1:53-60. The MCA and its lenticulostriatal branch are occluded proximally to the medial border of the olfactory tract with microbipolar coagulation. The wound is sutured, and animals returned to their home cage in a room warmed at 26° C. to 28° C. The temperature of the animals is maintained all the time with an automatic thermostat.

Evaluation Process. The efficacy of the Annexin II inhibitor is determined by mortality rate, weight gain, infarct volume, short and long term clinical and neurophysichological and behavioral (including feeding behavior) outcomes in surviving animals. Infarct volumes are assessed histologically (Knight et al., Stroke, 25, 1252, 1994, and Mintorovitch et al., Magn. Reson. Med. 18, 39, 1991). The staircase test (Montoya et al., J. Neurosci. Methods 36, 219, 1991) or the motor disability scale according to Bederson's method (Bederson et al., Stroke, 17, 472, 1986) is employed to evaluate the functional outcome following MCAO. The animals are followed for different time points, the longest one being two months. At each time point (24 h, 1 week, 3, 6, 8 weeks), animals are sacrificed and cardiac perfusion with 4% formaldehyde in PBS is performed. Brains are removed and serial coronal 200 μm sections are prepared for processing and paraffin embedding. The sections are stained with suitable dyes such as TCC. The infarct area is measured in these sections using a computerized image analyzer.

Utilization of the Annexin II inhibitor treatment as exemplified in the above animal models provides new possibilities for treatment of human brain injury, whether acute or chronic.

### Example 10

### Screening Systems

The Annexin II gene or polypeptide may be used in a screening assay for identifying and isolating compounds which modulate its activity and, in particular, compounds which modulate neurotoxic stress or neurodegenerative diseases. The compounds to be screened comprise inter alia substances such as small chemical molecules, antibodies, antisense oligonucleotides, antisense DNA or RNA molecules, polypeptides and dominant negatives, and expression vectors.

Many types of screening assays are known to those of ordinary skill in the art. The specific assay which is chosen depends to a great extent on the activity of the candidate gene or the polypeptide expressed thereby. Thus, if it is known that the expression product of a candidate gene has enzymatic activity, then an assay which is based on inhibition (or stimulation) of the enzymatic activity can be used. If the candidate polypeptide is known to bind to a ligand or other interactor, then the assay can be based on the inhibition of such binding or interaction. When the candidate gene is a known gene, then many of its properties can also be known, and these can be used to determine the best screening assay. If the candidate gene is novel, then some analysis and/or experimentation is appropriate in order to determine the best assay to be used to find inhibitors of the activity of that candidate gene. The analysis can involve a sequence analysis to find domains in the sequence which shed light on its activity.

As is well known in the art, the screening assays can be cell-based or non-cell-based. The cell-based assay is performed using eukaryotic cells such as HeLa cells, and such cell-based systems are particularly relevant in order to directly measure the activity of candidate genes which are anti-apoptotic functional genes, i.e., expression of the gene prevents apoptosis or otherwise prevents cell death in target cells. One way of running such a cell-based assay uses tetracycline-inducible (Tet-inducible) gene expression. Tet-inducible gene expression is well known in the art; see for example, Hofmann et al, 1996, Proc Natl Acad Sci 93(11):5185-5190.

Tet-inducible retroviruses have been designed incorporating the Self-inactivating (SIN) feature of a 3′ Ltr enhancer/promoter retroviral deletion mutant. Expression of this vector in cells is virtually undetectable in the presence of tetracycline or other active analogs. However, in the absence of Tet, expression is turned on to maximum within 48 hours after induction, with uniform increased expression of the whole population of cells that harbor the inducible retrovirus, thus indicating that expression is regulated uniformly within the infected cell population.

If the gene product of the candidate gene phosphorylates with a specific target protein, a specific reporter gene construct can be designed such that phosphorylation of this reporter gene product causes its activation, which can be followed by a color reaction. The candidate gene can be specifically induced, using the Tet-inducible system discussed above, and a comparison of induced versus non-induced genes provides a measure of reporter gene activation.

In a similar indirect assay, a reporter system can be designed that responds to changes in protein-protein interaction of the candidate protein. If the reporter responds to actual interaction with the candidate protein, a color reaction occurs.

One can also measure inhibition or stimulation of reporter gene activity by modulation of its expression levels via the specific candidate promoter or other regulatory elements. A specific promoter or regulatory element controlling the activity of a candidate gene is defined by methods well known in the art. A reporter gene is constructed which is controlled by the specific candidate gene promoter or regulatory elements. The DNA containing the specific promoter or regulatory agent is actually linked to the gene encoding the reporter. Reporter activity depends on specific activation of the promoter or regulatory element. Thus, inhibition or stimulation of the reporter is a direct assay of stimulation/inhibition of the reporter gene; see, for example, Komarov et al (1999), Science vol 285,1733-7 and Storz et al (1999) Analytical Biochemistry, 276, 97-104.

Various non-cell-based screening assays are also well within the skill of those of ordinary skill in the art. For example, if enzymatic activity is to be measured, such as if the candidate protein has a kinase activity, the target protein can be defined and specific phosphorylation of the target can be followed. The assay can involve either inhibition of target phosphorylation or stimulation of target phosphorylation, both types of assay being well known in the art; for example see Mohney et al (1998) J. Neuroscience 18, 5285 and Tang et al (1997) J. Clin. Invest. 100, 1180 for measurement of kinase activity. Specifically, assays for measuring the enzymatic activity of Annexin II are provided by Choi K S, Fitzpatrick S L, Filipenko N R, Fogg D K, Kassam G, Magliocco A M, Waisman D M: “Regulation of plasmin-dependent fibrin clot lysis by annexin II heterotetramer”, J Biol. Chem. 2001 Jul. 6; 276(27):25212-21 (Epub 2001 Apr. 23). Additionally, there is a possibility that Annexin II interacts with an enzyme and regulates its enzymatic activity through protein-protein interaction.

One can also measure in vitro interaction of a candidate polypeptide with interactors. In this screen, the candidate polypeptide is immobilized on beads. An interactor, such as a receptor ligand, is radioactively labeled and added. When it binds to the candidate polypeptide on the bead, the amount of radioactivity carried on the beads (due to interaction with the candidate polypeptide) can be measured. The assay indicates inhibition of the interaction by measuring the amount of radioactivity on the bead.

Any of the screening assays, according to the present invention, can include a step of identifying the chemical compound (as described above) or other species which tests positive in the assay and can also include the further step of producing as a medicament that which has been so identified. It is considered that medicaments comprising such compounds, or chemical analogs or homologs thereof, are part of the present invention. The use of any such compounds identified for inhibition or stimulation of apoptosis is also considered to be part of the present invention.

### Example 11

### Gene Therapy

The term “gene therapy” as used herein refers to the transfer of genetic material (e.g DNA or RNA) of interest into a host to treat or prevent a genetic or acquired disease or condition phenotype. The genetic material of interest encodes a product (e.g. a protein, polypeptide, peptide, functional RNA, antisense) the production of which in vivo is desired. For example, the genetic material of interest can encode a hormone, receptor, enzyme, polypeptide or peptide of therapeutic value. Alternatively, the genetic material of interest may encode a suicide gene. For a review see, in general, the text “Gene Therapy” (Advances in Pharmacology 40, Academic Press, 1997).

Gene therapy of the present invention can be carried out in vivo or ex vivo. Ex vivo gene therapy requires the isolation and purification of cells from a patient, the introduction of a therapeutic gene and the introduction of the genetically altered cells back into the patient. A replication-deficient virus such as a modified retrovirus can be used to introduce the therapeutic Annexin II cDNA or Annexin II antisense fragment into such cells. For example, mouse Moloney leukemia virus (MMLV) is a well-known vector in clinical gene therapy trials. See, e.g., Boris-Lauerie et al., Curr. Opin. Genet. Dev., 3, 102-109 (1993).

In contrast, in vivo gene therapy does not require isolation and purification of the cells from a patient. The therapeutic gene or fragment such as an antisense fragment is typically “packaged” for administration to a patient such as in liposomes or in a replication-deficient virus such as adenovirus as described by Berkner, K. L., in Curr. Top. Microbiol. Immunol., 158, 39-66 (1992) or adeno-associated virus (AAV) vectors as described by Muzyczka, N., in Curr. Top. Microbiol. Immunol., 158, 97-129 (1992) and U.S. Pat. No. 5,252,479. Another approach is administration of “naked DNA” in which the therapeutic gene or fragment such as an antisense fragment is directly injected into the bloodstream or muscle tissue. Still another approach is administration of “naked DNA” in which the therapeutic gene or fragment such as an antisense fragment is introduced into the target tissue by microparticle bombardment using gold particles coated with the DNA.

Gene therapy vectors can be delivered to a subject by, for example, intravenous injection, local administration (see U.S. Pat. No. 5,328,470) or by stereotactic injection (see e.g., Chen et al. (1994) PNAS 91:3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g. retroviral vectors, the pharmaceutical preparation can include one or more cells which produce the gene delivery system.

Cell types useful for gene therapy of the present invention include lymphocytes, hepatocytes, myoblasts, fibroblasts, and any cell of the eye such as retinal cells, epithelial and endothelial cells. Preferably the cells are T lymphocytes drawn from the patient to be treated, hepatocytes, any cell of the eye or respiratory or pulmonary epithelial cells. Transfection of pulmonary epithelial cells can occur via inhalation of a neubulized preparation of DNA vectors in liposomes, DNA-protein complexes or replication-deficient adenoviruses. See, e.g., U.S. Pat. No. 5,240,846. For a review of the subject of gene therapy, in general, see the text “Gene Therapy” (Advances in Pharmacology 40, Academic Press, 1997).

### Example 12

### Therapeutic Delivery of Antisense Fragments

In the practice of the invention, antisense fragments may be used. The length of an antisense fragment is preferably from about 9 to about 4,000 nucleotides, more preferably from about 20 to about 2,000 nucleotides, most preferably from about 50 to about 500 nucleotides.

In order to be effective, the antisense fragments of the present invention must travel across cell membranes. In general, antisense fragments have the ability to cross cell membranes, apparently by uptake via specific receptors. As the antisense fragments are single-stranded molecules, they are to a degree hydrophobic, which enhances passive diffusion through membranes. Modifications may be introduced to an antisense fragment to improve its ability to cross membranes. For instance, the AS molecule may be linked to a group which includes partially unsaturated aliphatic hydrocarbon chain and one or more polar or charged groups such as carboxylic acid groups, ester groups, and alcohol groups. Alternatively, AS fragments may be linked to peptide structures, which are preferably membranotropic peptides. Such modified AS fragments penetrate membranes more easily, which is critical for their function and may, therefore, significantly enhance their activity. Palmityl-linked oligonucleotides have been described by Gerster et al (1998): Quantitative analysis of modified antisense oligonucleotides in biological fluids using cationic nanoparticles for solid-phase extraction. Anal Biochem. 1998 Sep. 10; 262(2):177-84 Geraniol-linked oligonucleotides have been described by Shoji et al (1998): Enhancement of anti-herpetic activity of antisense phosphorothioate oligonucleotides 5′ end modified with geraniol. J Drug Target. 1998; 5(4):261-73. Oligonucleotides linked to peptides, e.g., membranotropic peptides, and their preparation have been described by Soukchareun et al (1998): Use of Nalpha-Fmoc-cysteine(S-thiobutyl) derivatized oligodeoxynucleotides for the preparation of oligodeoxynucleotide-peptide hybrid molecules. Bioconjug Chem. 1998 July-August; 9(4):466-75. Modifications of antisense molecules or other drugs that target the molecule to certain cells and enhance uptake of the oligonucleotide by said cells are described by Wang (1998).

The antisense oligonucleotides of the invention are generally provided in the form of pharmaceutical compositions. These compositions are for use by injection, topical administration, or oral uptake inter alia; see Example 8 Pharmacology and drug delivery.

The mechanism of action of antisense RNA and the current state of the art on use of antisense tools is reviewed in Kumar et al (1998): Antisense RNA: function and fate of duplex RNA in cells of higher eukaryotes. Microbiol Mol Biol Rev. 1998 December; 62(4):1415-34. There are reviews on the chemical (Crooke, 1995: Progress in antisense therapeutics. Hematol Pathol. 1995; 9(2):59-72.; Uhlmann et al, 1990), cellular (Wagner, 1994: Gene inhibition using antisense oligodeoxynucleotides. Nature. 1994 Nov. 24; 372(6504):333-5.) and therapeutic (Hanania, et al, 1995: Recent advances in the application of gene therapy to human disease. Am J. Med. 1995 November; 99(5):537-52.; Scanlon, et al, 1995: Oligonucleotide-mediated modulation of mammalian gene expression. FASEB J. 1995 October; 9(13):1288-96.; Gewirtz, 1993: Oligodeoxynucleotide-based therapeutics for human leukemias. Stem Cells. 1993 October; 11 Suppl 3:96-103) aspects of this rapidly developing technology. The use of antisense oligonucleotides in inhibition of Annexin receptor synthesis has been described by Yeh et al (1998): Inhibition of Annexin receptor synthesis by antisense oligonucleotides attenuates OP-1 action in primary cultures of fetal rat calvaria cells. J Bone Miner Res. 1998 December; 13(12):1870-9. The use of antisense oligonucleotides for inhibiting the synthesis of the voltage-dependent potassium channel gene Kv1.4 has been described by Meiri et al (1998) Memory and long-term potentiation (LTP) dissociated: normal spatial memory despite CA1 LTP elimination with Kv1.4 antisense. Proc Natl Acad Sci USA. 1998 Dec. 8; 95(25):15037-42. The use of antisense oligonucleotides for inhibition of the synthesis of Bcl-x has been described by Kondo et al (1998): Antisense telomerase treatment: induction of two distinct pathways, apoptosis and differentiation. FASEB J. 1998 July; 12(10):801-11. The therapeutic use of antisense drugs is discussed by Stix (1998): Shutting down a gene. Antisense drug wins approval. Sci Am. 1998 November; 279(5):46, 50; Flanagan (1998) Antisense comes of age. Cancer Metastasis Rev. 1998 June; 17(2):169-76; Guinot et al (1998) Antisense oligonucleotides: a new therapeutic approach Pathol Biol (Paris). 1998 May; 46(5):347-54, and references therein. Within a relatively short time, ample information has accumulated about the in vitro use of AS nucleotide sequences in cultured primary cells and cell lines as well as for in vivo administration of such nucleotide sequences for suppressing specific processes and changing body functions in a transient manner. Further, enough experience is now available from in vitro and in vivo in animal models and human clinical trials to predict human efficacy.

### Example 13

### Therapeutic Delivery of siRNA

Delivery systems aimed specifically at the enhanced and improved delivery of siRNA into mammalian cells have been developed, see, for example, Shen et al (FEBS letters 539: 111-114 (2003)), which describe an adenovirus-based vector for efficient delivery of siRNA into mammalian cells; Xia et al., Nature Biotechnology 20: 1006-1010 (2002), Reich et al., Molecular Vision 9: 210-216 (2003), Sorensen et al. (J. Mol. Biol. 327: 761-766 (2003), who devised injection-based systems for systemic delivery of siRNAs to adult mice, by cationic liposome-based intravenous injection and/or intraperitoneal injection, Lewis et al., Nature Genetics 32: 107-108 (2002) who developed a system for efficient delivery of siRNA into mice by rapid tail vain injection and Simeoni et al., Nucleic Acids Research 31, 11: 2717-2724 (2003), who delivered siRNA via the peptide based gene delivery system MPG, with the appropriate modifications.

siRNA has recently been successfully used for inhibition in primates; for further details see Tolentino et al., Retina 24(1) February 2004 I 132-138. Respiratory formulations for siRNA are described in U.S. patent application No. 2004/0063654 of Davis et al. Cholesterol-conjugated siRNAs (and other steroid and lipid conjugated siRNAs) can been used for delivery see Soutschek et al Nature 432: 173-177(2004) Therapeutic silencing of an endogenous gene by systemic administration of modified siRNAs; and Lorenz et al. Bioorg. Med. Chemistry. Lett. 14:4975-4977 (2004) Steroid and lipid conjugates of siRNAs to enhance cellular uptake and gene silencing in liver cells.

Additional methods which may be used for delivery of siRNAs are described in Examples 8 and 12.

1. A method of treating a patient suffering from a neurodegenerative disease or a central nervous system disorder, comprising administering to the patient a pharmaceutical composition comprising a therapeutically effective amount of an Annexin II inhibitor, so as to thereby treat the patient.

2. The method of claim 1, wherein the neurodegenerative disease is a stroke.

3. The method of claim 1, wherein the neurodegenerative disease is selected from the group consisting of hypertension, hypertensive cerebral vascular disease, systemic hypotension, Parkinson's disease, epilepsy, depression, ALS, Alzheimer's disease, Huntington's disease and HIV induced dementia.

4. A method for treating a patient who has suffered an injury to the central nervous system, comprising administering to the patient a pharmaceutical composition comprising a therapeutically effective amount of an Annexin II inhibitor in a dosage and over a period of time so as to thereby treat the patient.

5. The method of claim 4, wherein the injury is TBI.

6. The method of claim 4, wherein said injury is a spinal cord injury.

7. The method of claim 4, wherein the injury is selected from the group consisting of rupture of aneurysm, cardiac arrest, cardiogenic shock, septic shock, head trauma, seizure, and bleeding from a tumor.

8. The method of claim 1 wherein the Annexin II inhibitor is the small chemical compound sodium nitroprusside or Tyrphostin AG1024.

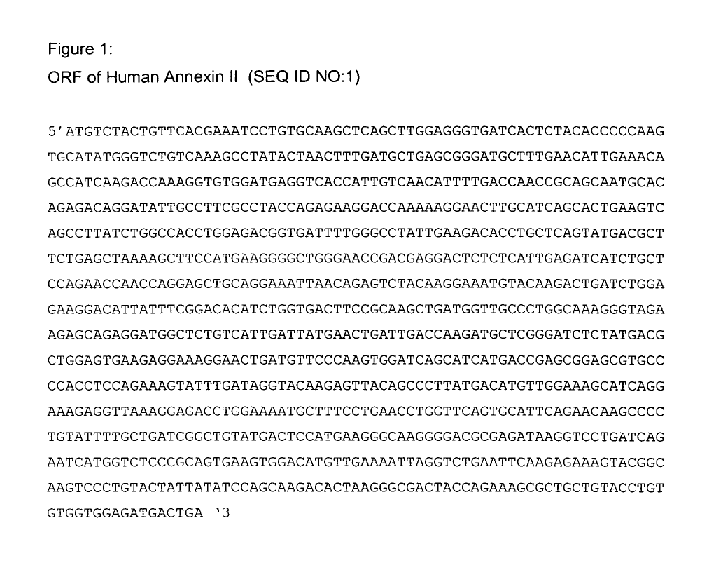

9. The method of claim 1 wherein the Annexin II inhibitor is an antisense polynucleotide comprising consecutive nucleotides having a sequence which is an antisense sequence to the sequence set forth in FIG. 1 (SEQ ID NO:1).

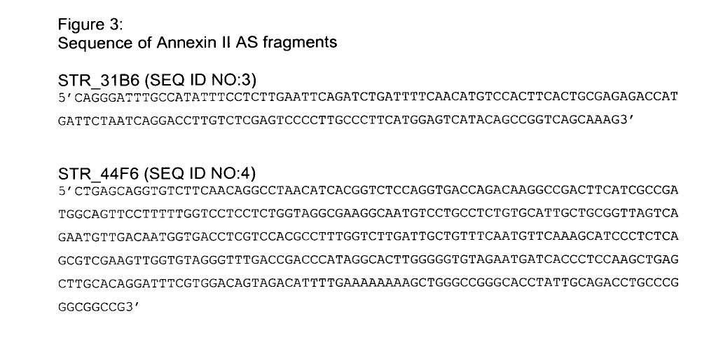

10. The method of claim 9 wherein the inhibitor is an antisense polynucleotide having a sequence set forth in FIG. 3 (SEQ ID NO:3 or SEQ ID NO:4).

11. The method of claim 1 wherein the Annexin II inhibitor is an siRNA.

12. The method of claim 11 wherein the siRNA has a sequence set forth in any one of Tables 1-3.

13. The method of claim 11 wherein the Annexin II inhibitor is an siRNA having a sequence set forth in Table 1, selected from the group consisting of SEQ ID NOs 12-16.

14. The method of claim 1 wherein the inhibitor has the structure:
5′ (N)ₓ-Z 3′ (antisense strand)
3′ Z′-(N′)_{y} 5′ (sense strand)
wherein each N and N′ is a ribonucleotide which may be modified or unmodified in its sugar residue and (N)ₓ and (N′)_{y} is an oligomer in which each consecutive N or N′ is joined to the next N or N′ by a covalent bond;
wherein each of x and y is an integer between 19 and 40;
wherein each of Z and Z′ may be present or absent, but if present is dTdT and is covalently attached at the 3′ terminus of the strand in which it is present;
and wherein the sequence of (N)ₓ comprises an antisense sequence to cDNA of Annexin II.

15. The method of claim 14 wherein the sequence of (N)ₓ comprises one or more of the antisense sequences present in Tables 1, 2 and 3.

16. The method of claim 1 wherein the Annexin II inhibitor is a polypeptide selected from the group consisting of a dominant negative peptide encoded by SEQ ID NO:5 or SEQ ID NO:6, peptide #41 of PCT patent application publication No. WO 200404/1844, or S-nitrosogluthathione.

17. The method of claim 1 wherein the Annexin II inhibitor is an antibody.

18. The method of claim 1 wherein the Annexin II inhibitor is a vector comprising a polynucleotide which encodes the inhibitor of any one of claims 9-16.

19. A compound having the structure
5′ (N)ₓ-Z 3′ (antisense strand)
3′ Z′-(N′)_{y} 5′ (sense strand)
wherein each N and N′ is a ribonucleotide which may be modified or unmodified in its sugar residue and (N)ₓ and (N′)_{y} is oligomer in which each consecutive N or N′ is joined to the next N or N′ by a covalent bond;
wherein each of x and y is an integer between 19 and 40;
wherein each of Z and Z′ may be present or absent, but if present is dTdT and is covalently attached at the 3′ terminus of the strand in which it is present;
and wherein the sequence of (N)ₓ comprises an antisense sequence to cDNA of Annexin II.

20. The compound of claim 19 wherein the sequence of (N)ₓ comprises one or more of the antisense sequences present in Tables 1, 2 and 3.
